(19) United States
(12) Patent Application Publication
(10) Pub. No.: US 2008/0181866 A1
(43) Pub. Date: Jul. 31, 2008

(54) Amido Anti-viral Compounds

(75) Inventors: Martin Robert Leivers, San Francisco, CA (US); Franz Ulrich Schmitz, Mill Valley, CA (US); Ronald Conrad Griffith, Escondido, CA (US); Christopher Don Roberts, Belmont, CA (US); Ali Dehghani Mohammad Abadi, Campbell, CA (US); Stephanie Anna Chan, San Francisco, CA (US); Roopa Rai, San Carlos, CA (US); Irina Slobodov, San Mateo, CA (US); Tony Loc Ton, Fremont, CA (US)

Correspondence Address: FOLEY & LARDNER LLP, 975 PAGE MILL ROAD, PALO ALTO, CA, 94304, US

(73) Assignee: Genelabs Technologies, Inc.

(21) Appl. No.: 11/943,545

(22) Filed: Nov. 20, 2007

Related U.S. Application Data
(60) Provisional application No. 60/860,545, filed on Nov. 21, 2006.
(60) Provisional application No. 60/943,530, filed on Jun. 12, 2007.

(51) Int. Cl. A61K 38/21; C07D 277/06; A61K 31/426; A61P 31/12; C07D 401/14; A61K 31/4439

(52) U.S. Cl. 424/85.4; 548/201; 514/365; 546/270.4; 514/342

(57) ABSTRACT

Disclosed are compounds, stereoisomers, tautomers, pharmaceutically acceptable salts, or prodrugs thereof of having Formula (I), their preparation, use, and compositions thereof for treating an infection mediated at least in part by a virus in the Flaviviridae family of viruses, wherein A, R³, X, V, W, T, Z, R, Y¹, and p are as defined herein.

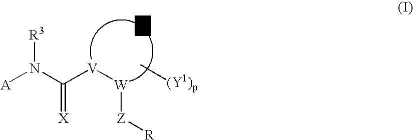

### CROSS-REFERENCE TO RELATED APPLICATIONS

This application claims the benefit under 35 U.S.C. §119(e) to U.S. provisional applications Ser. No. 60/860,545 filed on Nov. 21, 2006 and to Ser. No. 60/943,530 filed on Jun. 12, 2007, which are both incorporated herein by reference in their entirety.

### BACKGROUND OF THE INVENTION

1. Field of the Invention

The invention relates to the field of pharmaceutical chemistry, in particular to compounds, their preparation, compositions, and uses thereof for treating viral infections in patients mediated, at least in part, by a virus in the Flaviviridae family of viruses.

### REFERENCES

The following publications are cited in this application as superscript numbers:
1. Szabo, et al., Pathol. Oncol. Res. 2003, 9:215-221.
2. Hoofnagle J H, Hepatology 1997, 26:15S-20S.
3. Thomson B J and Finch R G, Clin Microbial Infect. 2005, 11:86-94.
4. Moriishi K and Matsuura Y, Antivir. Chem. Chemother. 2003, 14:285-297.
5. Fried, et al. N. Engl. J. Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, et al., Ann. Rep. Med. Chem. 39, 223-237, 2004.
9. Watashi, et al, Molecular Cell, 19, 111-122, 2005
10. Horsmans, et al, Hepatology, 42, 724-731, 2005

All of the above publications are herein incorporated by reference in their entirety to the same extent as if each individual publication was specifically and individually indicated to be incorporated by reference in its entirety.

2. State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load 5 and there is a clear need for more effective antiviral therapy of HCV infection.

A number of approaches are being pursuit to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4A protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

Notwithstanding the above, the discovery of new compounds active against one or more members of the Flaviviridae family of viruses would be beneficial particularly in view of the difficulty currently faced in treating diseases mediated, at least in part, by one or more of such viruses.

### SUMMARY OF THE INVENTION

This invention is directed to compounds, their preparation, compositions, prodrugs, and uses thereof for treating viral infections mediated, at least in part, by a virus in the Flaviviridae family of viruses. In one embodiment, provided is compound of Formula (I) or a stereoisomer, tautomer, pharmaceutically acceptable salt, or prodrug thereof, wherein:

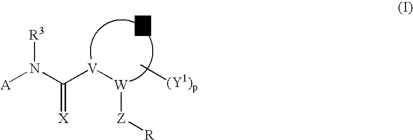

A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
R³ is selected from the group consisting of hydrogen, alkyl, substituted alkyl, cycloalkyl, and substituted cycloalkyl;
X is O or S;
T is C₂-C₆ alkylene or C₁-C₅ heteroalkylene and forms a 4-8 membered ring with V and W;
V and W are both CH, or one of V or W is CH and the other of V or W is N;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In one embodiment provided is a pharmaceutical composition comprising a pharmaceutically acceptable carrier and a therapeutically effective amount of a compound, stereoisomer, tautomer, pharmaceutically acceptable salt, or prodrug thereof of Formula (I).

In one embodiment provided is a method for treating a viral infection in a patient mediated at least in part by a virus in the Flaviviridae family of viruses, comprising administering to said patient a composition of Formula (I). In some aspects, the viral infection is mediated by hepatitis C virus.

### DETAILED DESCRIPTION

### Definitions

As used herein, the following definitions shall apply unless otherwise indicated.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy substitution is not attached to an acetylenic carbon atom.

“C₂-C₆ alkylene” refers to divalent straight chain alkyl groups having from 1 to 6 carbons.

“C₁-C₅ heteroalkylene” refers to alkylene groups where one or two —CH₂— groups are replaced with —S—, or —O— to give a heteroalkylene having one to five carbons provided that the heteroalkylene does not contain an —O—O—, —S—O—, or —S—S— group. When a —S— group is present, the term “C₁-C₅ heteroalkylene” includes the corresponding oxide metabolites —S(O)— and —S(O)₂—.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O)substituted alkyl, —NR⁴⁷C(O)cycloalkyl, —NR⁴⁷C(O)substituted cycloalkyl, —NR⁴⁷C(O)cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O)heterocyclic, and —NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR⁴⁷C(O)O-alkyl, —NR⁴⁷C(O)O-substituted alkyl, —NR⁴⁷C(O)O-alkenyl, —NR⁴⁷C(O)O-substituted alkenyl, —NR⁴⁷C(O)O-alkynyl, —NR⁴⁷C(O)O-substituted alkynyl, —NR⁴⁷C(O)O-aryl, —NR⁴⁷C(O)O-substituted aryl, —NR⁴⁷C(O)O-cycloalkyl, —NR⁴⁷C(O)O-substituted cycloalkyl, —NR⁴⁷C(O)O-cycloalkenyl, —NR⁴⁷C(O)O-substituted cycloalkenyl, —NR⁴⁷C(O)O-heteroaryl, —NR⁴⁷C(O)O-substituted heteroaryl, —NR⁴⁷C(O)O-heterocyclic, and —NR⁴⁷C(O)O-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring (e.g. fluorenyl). Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused, bridged, and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O—).

“Spiro ring systems” refers to bicyclic ring systems that have only a single ring atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thioxo” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Metabolite” refers to any derivative produced in a subject after administration of a parent compound. The metabolite may be produced from the parent compound by various biochemical transformations in the subject such as, for example, oxidation, reduction, hydrolysis, or conjugation. Metabolites include, for example, oxides and demethylated derivatives.

“Prodrug” refers to art recognized modifications to one or more functional groups which functional groups are metabolized in vivo to provide a compound of this invention or an active metabolite thereof. Such functional groups are well known in the art including acyl groups for hydroxyl and/or amino substitution, esters of mono-, di- and tri-phosphates wherein one or more of the pendent hydroxyl groups have been converted to an alkoxy, a substituted alkoxy, an aryloxy or a substituted aryloxy group, and the like.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate [see Stahl and Wermuth, eds., “Handbook of Pharmaceutically Acceptable Salts”, (2002), Verlag Helvetica Chimica Acta, Zürich, Switzerland, for an extensive discussion of pharmaceutical salts, their selection, preparation, and use].

“Therapeutically effective amount” is an amount sufficient to treat a specified disorder or disease.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, the present invention provides a compound of Formula (I) or a stereoisomer, tautomer, pharmaceutically acceptable salt, or prodrug thereof, wherein:

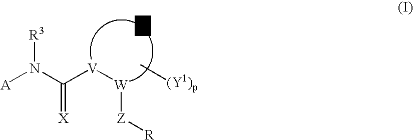

A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
R³ is selected from the group consisting of hydrogen, alkyl, substituted alkyl, cycloalkyl, and substituted cycloalkyl;
X is O or S;
T is C₂-C₆ alkylene or C₁-C₅ heteroalkylene and forms a 4-8 membered ring with V and W;
V and W are both CH, or one of V or W is CH and the other of V or W is N;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In one aspect, X is O.

In one aspect, R³ is hydrogen.

In one aspect, A is selected from the group consisting of

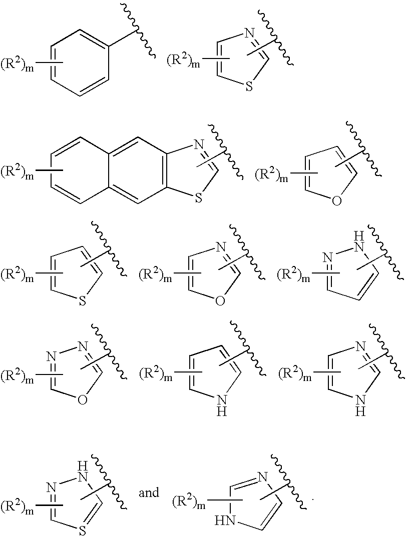

In one embodiment, provided is a compound of Formula (II) or a stereoisomer, tautomer, pharmaceutically acceptable salt, or prodrug thereof, wherein:

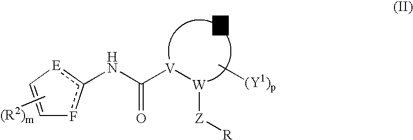

one of E or F is —N═ and the other of E or F is —O—, —S—, or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
T is C₂-C₆ alkylene or C₁-C₅ heteroalkylene and forms a 4-8 membered ring with V and W;
V and W are both CH, or one of V or W is CH and the other of V or W is N;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In some aspects of the compound, stereoisomer, tautomer, pharmaceutically acceptable salt, or prodrug thereof of Formula (II) and (III), V is C and W is N.

In one embodiment, provided is a compound of Formula (III) or a stereoisomer, tautomer, pharmaceutically acceptable salt, or prodrug thereof, wherein:

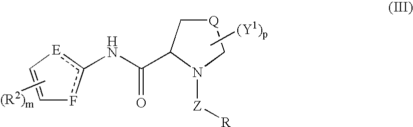

one of E or F is —N═ and the other of E or F is —O—, —S—, or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
Q is selected from the group consisting of CH₂, CH(Y¹), C(Y¹)(Y¹), S, and O;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In some aspects of the compounds, stereoisomer, tautomer, pharmaceutically acceptable salt, or prodrug thereof of Formula (III), Q is S, CH₂, or O.

In some aspects of the compounds, stereoisomer, tautomer, pharmaceutically acceptable salt, or prodrug thereof of Formula (I)-(III), at least one of R² is R⁴—L- wherein R⁴ is selected from the group consisting of aryl, substituted aryl, heteroaryl, substituted heteroaryl, heterocyclic, and substituted heterocyclic; and L, defined in the R⁴-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R⁴ is heterocyclic or substituted heterocyclic. In some aspects, L is a bond.

In some aspects, R⁴ is substituted phenyl. In some such aspects, said phenyl is substituted with one to three groups independently selected from alkyl, substituted alkyl, alkenyl, substituted alkenyl, alkoxy, substituted alkoxy, aryloxy, substituted aryloxy, alkylthio, substituted alkyl thio, acyl, acylamino, acyloxy, amino, substituted amino, aminocarbonyl, aminocarbonylamino, aminocarbonyloxy, aryl, substituted aryl, carboxyl, carboxyl ester, cyano cycloalkyl, substituted cycloalkyl, halo, hydroxy, heteroaryl, substituted heteroaryl, heterocyclic, substituted heterocyclic, nitro, thiol, alkylthio, and substituted alkylthio. In some aspects, at least one substitutent is cycloalkyl-C(O)NH—.

In some aspects, R⁴ is phenyl substituted with at least one group substituent selected from cyclopropyl-C(O)NH—, phenyl-C(O)NH—, cyclopentyl-C(O)NH—, 4-chlorophenyl-C(O)NH—, 4-chlorophenyl-C(O)NH—, methyl-C(O)NH—, methylamino, 4-methylphenyl-SO₂NH—, amino, ethyl-C(O)NH—, bromo, methoxy, methyl-SO₂NH—, chloro, phenyl-SO₂NH—, methyl-C(O)NH—, methyl-C(O)—, fluoro, methyl, ethyl, propyl, 4-fluorophenyl, nitro, phenyl, 4-bromobenzyloxy, cyclohexyl, isopropyl, tert-butyl, 4-methylpentyloxymethyl, NH₂C(O)—, hydroxy, cyclohexyl-NHC(O)CH₂S—, allyl, ethoxycarbonylmethylthio, dimethylamino, 3-nitro-phenyl, isobutyl, propoxy, butoxymethyl, butyl-C(O)NH—, methyl-NHC(O)—, ethyl-NHC(O)—, (2-oxo-hexahydro-thieno[3,4-d]imidazol-4-yl)-butyl-C(O)NH—, cyclopropyl-NHC(O)—, cyclohexyl-NHC(O)—, cyclopentyl-NHC(O)—, propyl, isobutyl, carboxy, pentyl-C(O)NH—, phenylamino-C(O)—, cylopropylamino-C(O)—, isopropylamino-C(O)—, and ethylamino-C(O)—.

In other aspects, T is —CH₂CH₂CH₂—.

In still other aspects provided is a compound of formula (IIIa) or a stereoisomer, tautomer, pharmaceutically acceptable salt, or prodrug thereof, wherein:

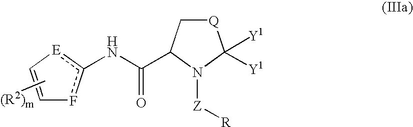

two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing Q form a spiro ring system; and
R², m, E, F, Q, Z, R, and Y² are as defined for Formula (III).

In one embodiment, provided is a compound of Formula (IV) or a stereoisomer, tautomer, pharmaceutically acceptable salt, or prodrug thereof, wherein:

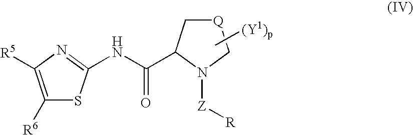

R⁵ is selected from the group consisting of substituted cycloalkyl, substituted phenyl, substituted heterocyclic, and substituted heteroaryl;
R⁶ is independently selected from the group consisting of hydrogen, alkyl, substituted alkyl, and halo;
Q is selected from the group consisting of CH₂, CH(Y¹), C(Y¹)(Y¹), S, and O;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

In some aspects of the compounds, stereoisomer, tautomer, pharmaceutically acceptable salt, or prodrug thereof of Formula (IV), Q is S, CH₂, or O.

In other aspects, R⁵ is substituted phenyl. In some such aspects, said phenyl is substituted with one to three groups independently selected from alkyl, substituted alkyl, alkenyl, substituted alkenyl, alkoxy, substituted alkoxy, aryloxy, substituted aryloxy, alkylthio, substituted alkyl thio, acyl, acylamino, acyloxy, amino, substituted amino, aminocarbonyl, aminocarbonylamino, aminocarbonyloxy, aryl, substituted aryl, carboxyl, carboxyl ester, cyano cycloalkyl, substituted cycloalkyl, halo, hydroxy, heteroaryl, substituted heteroaryl, heterocyclic, substituted heterocyclic, nitro, thiol, alkylthio, and substituted alkylthio. In some aspects, at least one substitutent is cycloalkyl-C(O)NH—.

In some aspects, R⁵ is phenyl substituted with at least one group substituent selected from cyclopropyl-C(O)NH—, phenyl-C(O)NH—, cyclopentyl-C(O)NH—, 4-chlorophenyl-C(O)NH—, 4-chlorophenyl-C(O)NH—, methyl-C(O)NH—, methylamino, 4-methylphenyl-SO₂NH—, amino, ethyl-C(O)NH—, bromo, methoxy, methyl-SO₂NH—, chloro, phenyl-SO₂NH—, methyl-C(O)NH—, methyl-C(O)—, fluoro, methyl, ethyl, propyl, 4-fluorophenyl, nitro, phenyl, 4-bromobenzyloxy, cyclohexyl, isopropyl, tert-butyl, 4-methylpentyloxymethyl, NH₂C(O)—, hydroxy, cyclohexyl-NHC(O)CH₂S—, allyl, ethoxycarbonylmethylthio, dimethylamino, 3-nitro-phenyl, isobutyl, propoxy, butoxymethyl, butyl-C(O)NH—, methyl-NHC(O)—, ethyl-NHC(O)—, (2-oxo-hexahydro-thieno[3,4-d]imidazol-4-yl)-butyl-C(O)NH—, cyclopropyl-NHC(O)—, cyclohexyl-NHC(O)—, cyclopentyl-NHC(O)—, propyl, isobutyl, carboxy, pentyl-C(O)NH—, phenylamino-C(O)—, cylopropylamino-C(O)—, isopropylamino-C(O)—, and ethylamino-C(O)—.

In other aspects, R⁶ is hydrogen.

In other aspects, R is OCH₂R¹ and R¹ is phenyl or substituted phenyl. In still other aspects R¹ is pyridyl or substituted pyridyl.

In other aspects, Z is C(O)R, R is OCH₂R¹, R¹ is phenyl or substituted phenyl, and R⁵ is substituted phenyl. In still other aspects R¹ is pyridyl or substituted pyridyl.

In other aspects of the compounds of Formula IV, p is 2 and two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing Q form a spiro ring system.

In some aspects of the compounds, stereoisomer, tautomer, pharmaceutically acceptable salt, or prodrug thereof of Formula (I)-(III), Z is C(O).

In other aspects, R is OCH₂R¹ and R¹ is phenyl or substituted phenyl. In still other aspects R¹ is pyridyl or substituted pyridyl.

In other aspects, Z and R together are C(O)R, R is OCH₂R¹, R¹ is phenyl or substituted phenyl, and R⁵ is substituted phenyl. In still other aspects R¹ is pyridyl or substituted pyridyl.

In other aspects, p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

In other aspects Y¹ is phenyl, pyridyl, substituted phenyl, or substituted pyridyl.

In other aspects Y¹ is pyridin-2-yl, pyridin-3-yl, pyridin-4-yl, 3-fluoro-pyridin-4-yl-, 2-hydroxy-pyridin-4-yl, tetrahydro-pyran-4-ylmethyl, phenyl, 2-fluoro-phenyl, 3-fluoro-phenyl, 4-fluorophenyl, 3-carboxy-phenyl, 4-carboxy-phenyl, 3-methoxycarbonyl-phenyl, 4-methoxycarbonyl-phenyl, 2-methoxy-phenyl, 3-methoxy-phenyl, 4-methoxy-phenyl, phenylmethyl, quinolin-4-yl, thiazol-2-yl, 3-cyano-phenyl, 4-cyano-phenyl, piperidin-3-yl-, piperidin-4-yl, pyrimidin-5-yl-, tetrahydro-pyran-4-yl, 2-chloro-pyridin-4-yl, cyclohexyl, oxazol-5-yl, 4-morpholin-4-ylmethyl-phenyl, 1-methyl-1H-imidazol-2-yl, or oxazol-2-yl.

In other aspects p is 2 and two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing T, V, and W form a spiro ring system.

In still other aspects Y¹ is selected from the corresponding groups in Table 1.

In yet other embodiments, the present invention provides a compound, stereoisomer, tautomer, or a pharmaceutically acceptable salt thereof selected from Table 1.

TABLE 1

| Compound | Structure | Name |
|---|---|---|
| 7001 | 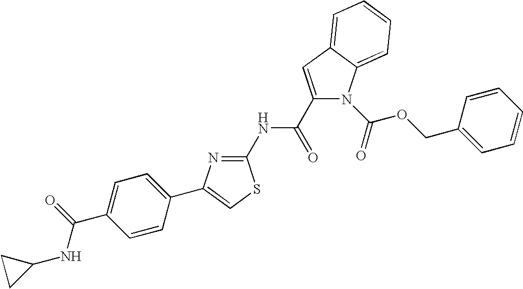 | 2-[4-(4-Cyclopropylcarbamoyl-phenyl)-thiazol-2-ylcarbamoyl]-indole-1-carboxylic acid benzyl ester |
| 7002 | 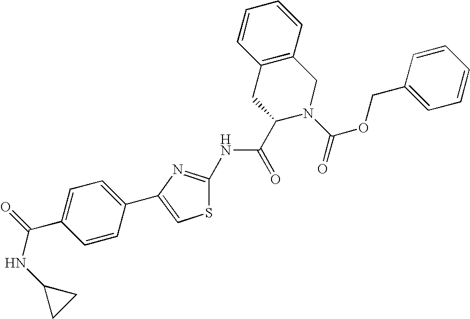 | 3-[4-(4-Cyclopropylcarbamoyl-phenyl)-thiazol-2-ylcarbamoyl]-3,4-dihydro-1H-isoquinoline-2-carboxylic acidbenzyl ester |
| 7003 | 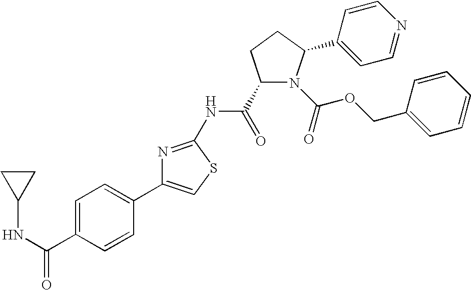 | 2-[4-(4-Cyclopropylcarbamoyl-phenyl)-thiazol-2-ylcarbamoyl]-5-pyridin-4-yl-pyrrolidine-1-carboxylic acidbenzyl ester |
| 7004 | 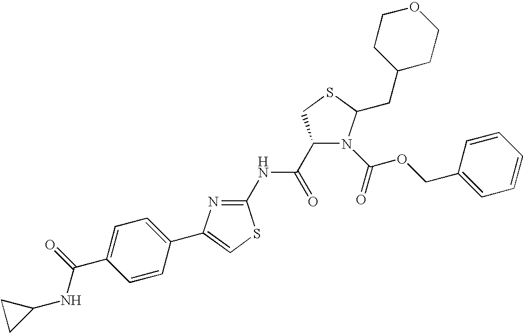 | 4-[4-(4-Cyclopropylcarbamoyl-phenyl)-thiazol-2-ylcarbamoyl]-2-(tetrahydro-pyran-4-ylmethyl)-thiazolidine-3-carboxylic acid benzyl ester |
| 7005 | 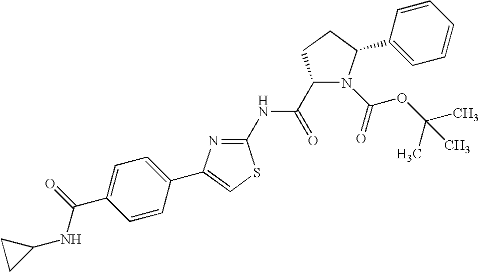 | 2-[4-(4-Cyclopropylcarbamoyl-phenyl)-thiazol-2-ylcarbamoyl]-5-phenyl-pyrrolidine-1-carboxylic acidtert-butyl ester |
| 7006 | 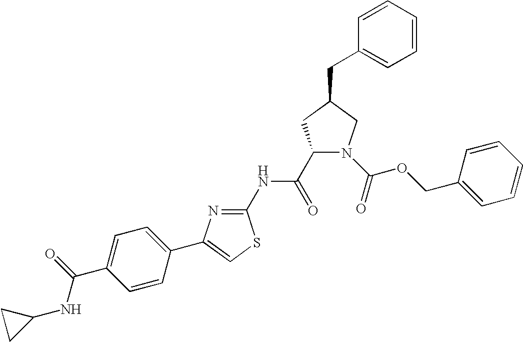 | 4-Benzyl-2-[4-(4-cyclopropylcarbamoyl-phenyl)-thiazol-2-ylcarbamoyl]-pyrrolidine-1-carboxylic acidbenzyl ester |
| 7007 | 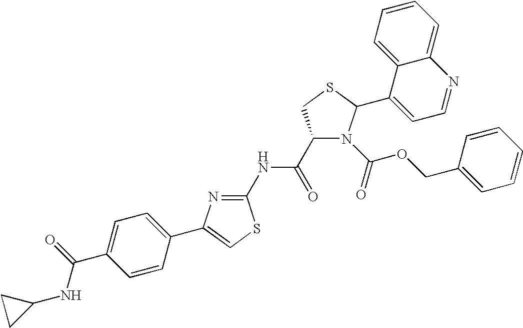 | 4-[4-(4-Cyclopropylcarbamoyl-phenyl)-thiazol-2-ylcarbamoyl]-2-quinolin-4-yl-thiazolidine-3-carboxylic acidbenzyl ester |
| 7008 | 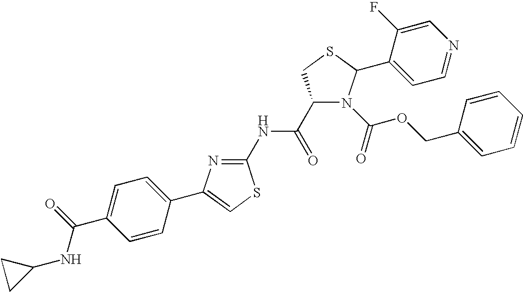 | 4-[4-(4-Cyclopropylcarbamoyl-phenyl)-thiazol-2-ylcarbamoyl]-2-(3-fluoro-pyridin-4-yl)-thiazolidine-3-carboxylic acid benzyl ester |
| 7009 | 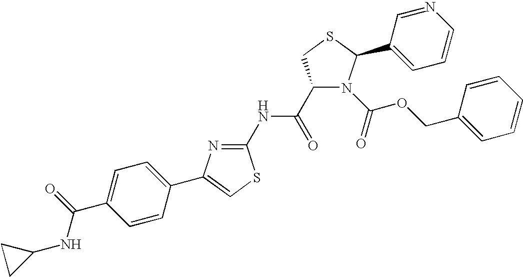 | 4-[4-(4-Cyclopropylcarbamoyl-phenyl)-thiazol-2-ylcarbamoyl]-2-pyridin-3-yl-thiazolidine-3-carboxylic acidbenzyl ester |
| 7010 | 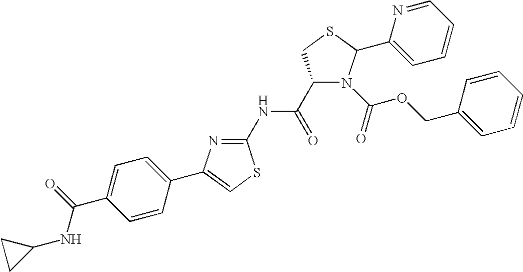 | 4-[4-(4-Cyclopropylcarbamoyl-phenyl)-thiazol-2-ylcarbamoyl]-2-pyridin-2-yl-thiazolidine-3-carboxylic acidbenzyl ester |
| 7011 | 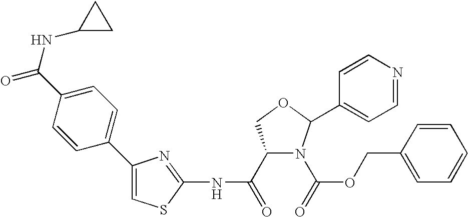 | 4-[4-(4-Cyclopropylcarbamoyl-phenyl)-thiazol-2-ylcarbamoyl]-2-pyridin-4-yl-oxazolidine-3-carboxylic acidbenzyl ester |
| 7012 | 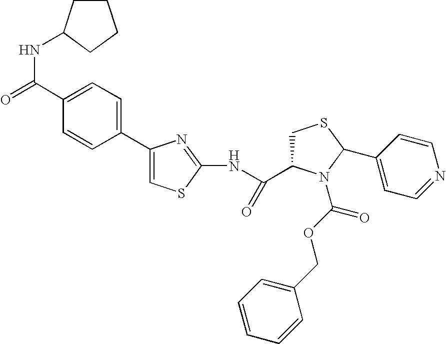 | 4-[4-(4-Cyclopentylcarbamoyl-phenyl)-thiazol-2-ylcarbamoyl]-2-pyridin-4-yl-thiazolidine-3-carboxylic acidbenzyl ester |
| 7013 | 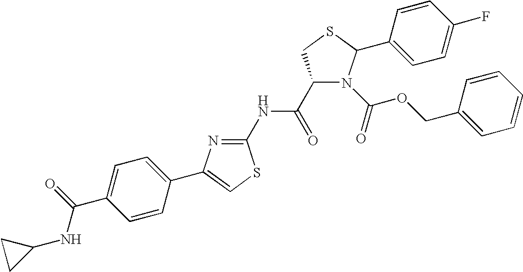 | 4-[4-(4-Cyclopropylcarbamoyl-phenyl)-thiazol-2-ylcarbamoyl]-2-(4-fluoro-phenyl)-thiazolidine-3-carboxylic acid benzyl ester |
| 7014 | 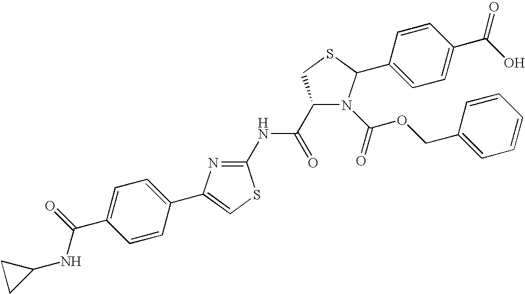 | 2-(4-Carboxy-phenyl)-4-[4-(4-cyclopropylcarbamoyl-phenyl)-thiazol-2-ylcarbamoyl]-thiazolidine-3-carboxylic acidbenzyl ester |
| 7015 | 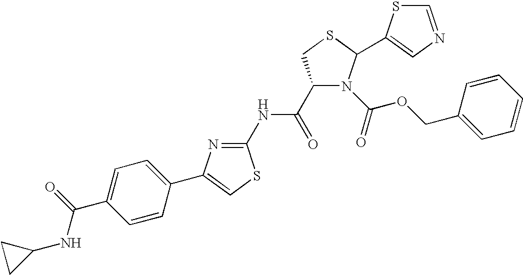 | 4-[4-(4-Cyclopropylcarbamoyl-phenyl)-thiazol-2-ylcarbamoyl]-4,5-dihydro-[2,5′]bithiazolyl-3-carboxylicacid benzyl ester |
| 7016 | 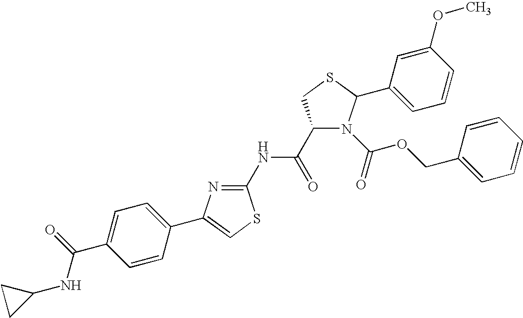 | 4-[4-(4-Cyclopropylcarbamoyl-phenyl)-thiazol-2-ylcarbamoyl]-2-(3-methoxy-phenyl)-thiazolidine-3-carboxylic acid benzyl ester |
| 7017 | 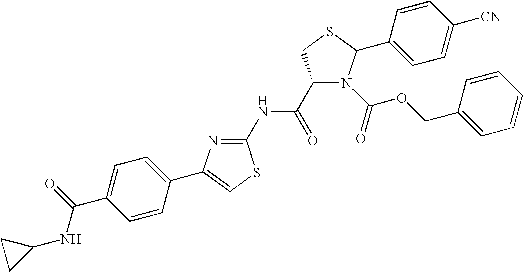 | 2-(4-Cyano-phenyl)-4-[4-(4-cyclopropylcarbamoyl-phenyl)-thiazol-2-ylcarbamoyl]-thiazolidine-3-carboxylic acidbenzyl ester |
| 7018 | 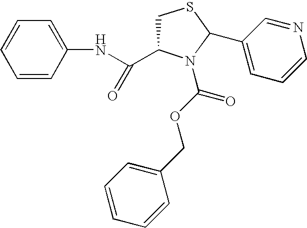 | 4-Phenylcarbamoyl-2-pyridin-3-yl-thiazolidine-3-carboxylicacid benzyl ester |
| 7019 | 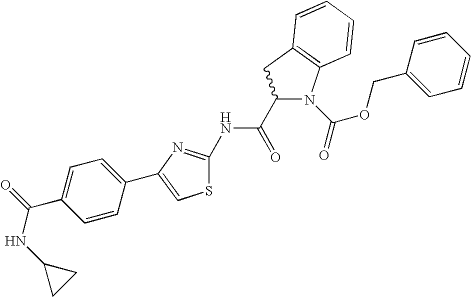 | 2-[4-(4-Cyclopropylcarbamoyl-phenyl)-thiazol-2-ylcarbamoyl]-2,3-dihydro-indole-1-carboxylic acid benzylester |
| 7020 | 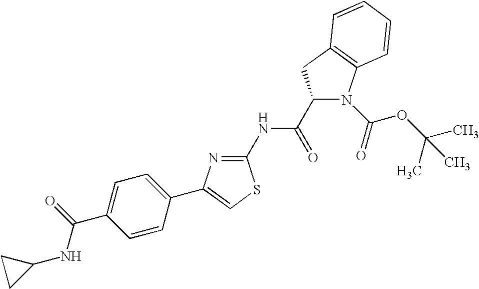 | 2-[4-(4-Cyclopropylcarbamoyl-phenyl)-thiazol-2-ylcarbamoyl]-2,3-dihydro-indole-1-carboxylic acid tert-butyl ester |
| 7021 | 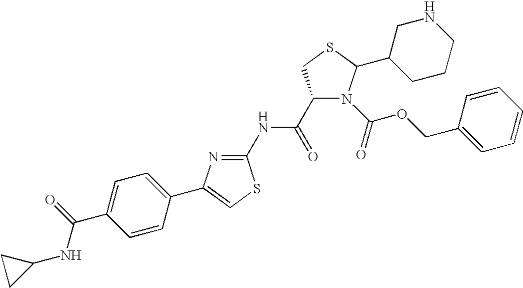 | 4-[4-(4-Cyclopropylcarbamoyl-phenyl)-thiazol-2-ylcarbamoyl]-2-piperidin-3-yl-thiazolidine-3-carboxylic acidbenzyl ester |
| 7022 | 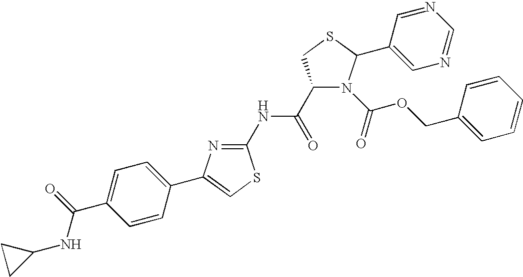 | 4-[4-(4-Cyclopropylcarbamoyl-phenyl)-thiazol-2-ylcarbamoyl]-2-pyrimidin-5-yl-thiazolidine-3-carboxylic acidbenzyl ester |
| 7023 | 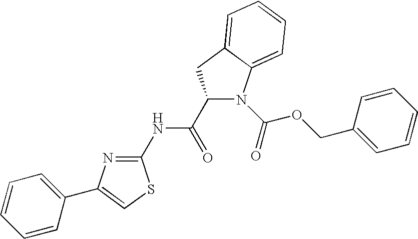 | 2-(4-Phenyl-thiazol-2-ylcarbamoyl)-2,3-dihydro-indole-1-carboxylic acid benzylester |
| 7024 | 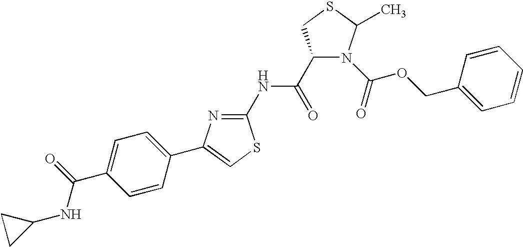 | 4-[4-(4-Cyclopropylcarbamoyl-phenyl)-thiazol-2-ylcarbamoyl]-2-methyl-thiazolidine-3-carboxylic acidbenzyl ester |
| 7025 | 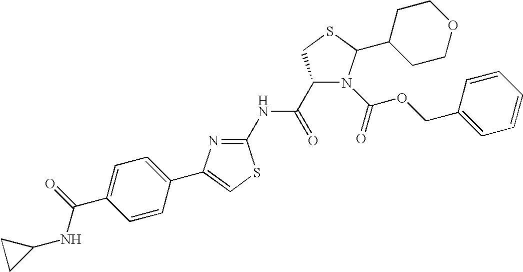 | 4-[4-(4-Cyclopropylcarbamoyl-phenyl)-thiazol-2-ylcarbamoyl]-2-(tetrahydro-pyran-4-yl)-thiazolidine-3-carboxylic acid benzyl ester |
| 7026 | 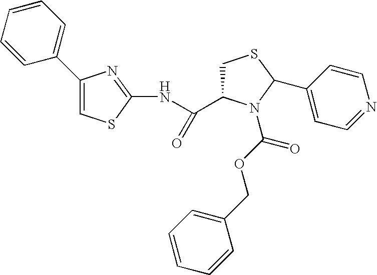 | 4-(4-Phenyl-thiazol-2-ylcarbamoyl)-2-pyridin-4-yl-thiazolidine-3-carboxylic acidbenzyl ester |
| 7027 | 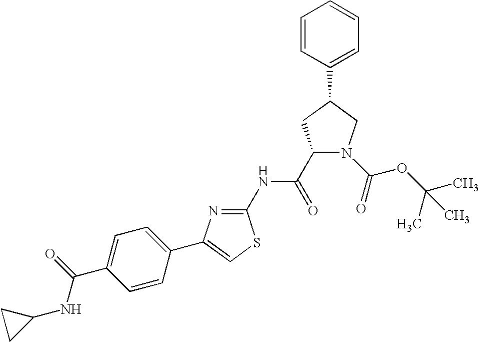 | 2-[4-(4-Cyclopropylcarbamoyl-phenyl)-thiazol-2-ylcarbamoyl]-4-phenyl-pyrrolidine-1-carboxylic acidtert-butyl ester |
| 7028 | 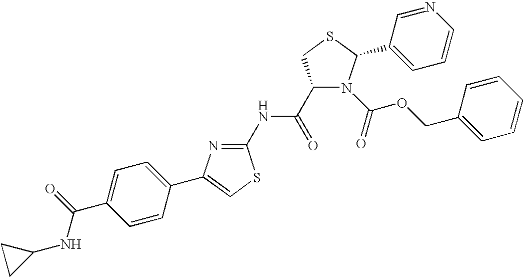 | 4-[4-(4-Cyclopropylcarbamoyl-phenyl)-thiazol-2-ylcarbamoyl]-2-pyridin-3-yl-thiazolidine-3-carboxylic acidbenzyl ester |
| 7029 | 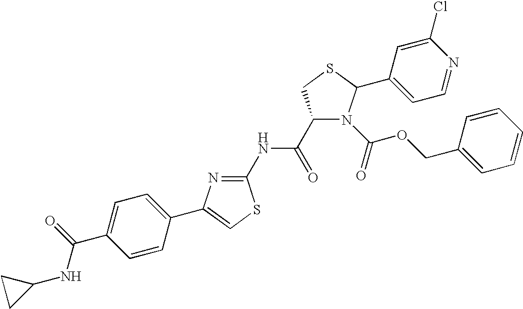 | 2-(2-Chloro-pyridin-4-yl)-4-[4-(4-cyclopropylcarbamoyl-phenyl)-thiazol-2-ylcarbamoyl]-thiazolidine-3-carboxylic acid benzyl ester |
| 7030 | 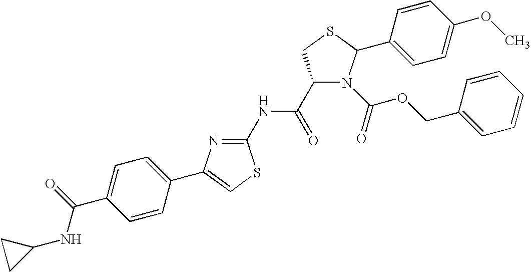 | 4-[4-(4-Cyclopropylcarbamoyl-phenyl)-thiazol-2-ylcarbamoyl]-2-(4-methoxy-phenyl)-thiazolidine-3-carboxylic acid benzyl ester |
| 7031 | 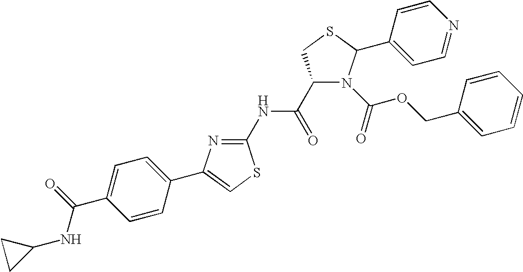 | 4-[4-(4-Cyclopropylcarbamoyl-phenyl)-thiazol-2-ylcarbamoyl]-2-pyridin-4-yl-thiazolidine-3-carboxylic acidbenzyl ester |
| 7032 | 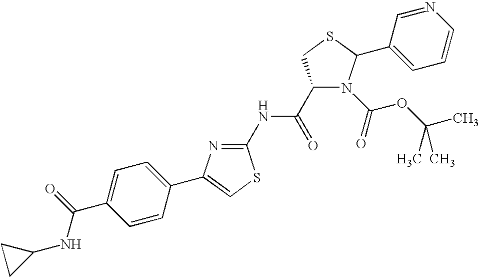 | 4-[4-(4-Cyclopropylcarbamoyl-phenyl)-thiazol-2-ylcarbamoyl]-2-pyridin-3-yl-thiazolidine-3-carboxylic acidtert-butyl ester |
| 7033 | 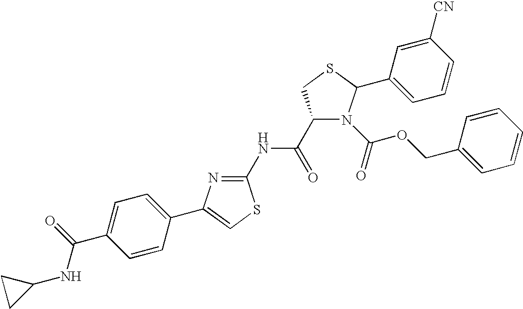 | 2-(3-Cyano-phenyl)-4-[4-(4-cyclopropylcarbamoyl-phenyl)-thiazol-2-ylcarbamoyl]-thiazolidine-3-carboxylic acidbenzyl ester |
| 7034 | 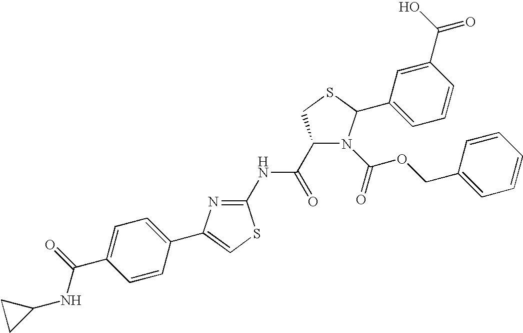 | 2-(3-Carboxy-phenyl)-4-[4-(4-cyclopropylcarbamoyl-phenyl)-thiazol-2-ylcarbamoyl]-thiazolidine-3-carboxylic acidbenzyl ester |
| 7035 | 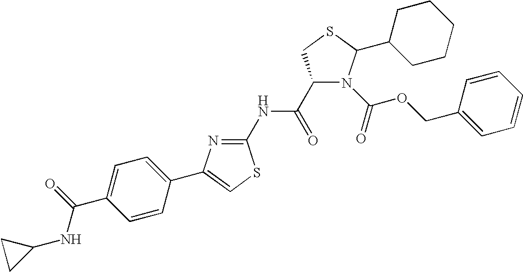 | 2-Cyclohexyl-4-[4-(4-cyclopropylcarbamoyl-phenyl)-thiazol-2-ylcarbamoyl]-thiazolidine-3-carboxylic acidbenzyl ester |
| 7036 | 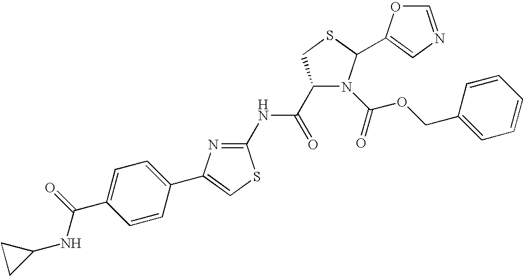 | 4-[4-(4-Cyclopropylcarbamoyl-phenyl)-thiazol-2-ylcarbamoyl]-2-oxazol-5-yl-thiazolidine-3-carboxylic acidbenzyl ester |
| 7037 | 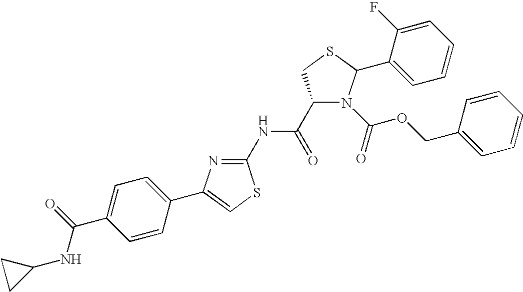 | 4-[4-(4-Cyclopropylcarbamoyl-phenyl)-thiazol-2-ylcarbamoyl]-2-(2-fluoro-phenyl)-thiazolidine-3-carboxylic acid benzyl ester |
| 7038 | 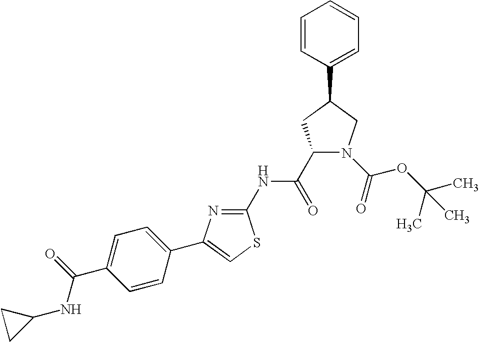 | 2-[4-(4-Cyclopropylcarbamoyl-phenyl)-thiazol-2-ylcarbamoyl]-4-phenyl-pyrrolidine-1-carboxylic acidtert-butyl ester |
| 7039 | 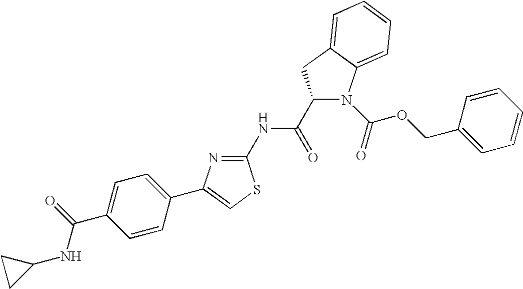 | 2-[4-(4-Cyclopropylcarbamoyl-phenyl)-thiazol-2-ylcarbamoyl]-2,3-dihydro-indole-1-carboxylic acid benzylester |
| 7040 | 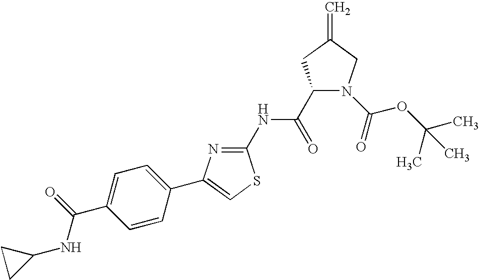 | 2-[4-(4-Cyclopropylcarbamoyl-phenyl)-thiazol-2-ylcarbamoyl]-4-methylene-pyrrolidine-1-carboxylic acidtert-butyl ester |
| 7041 | 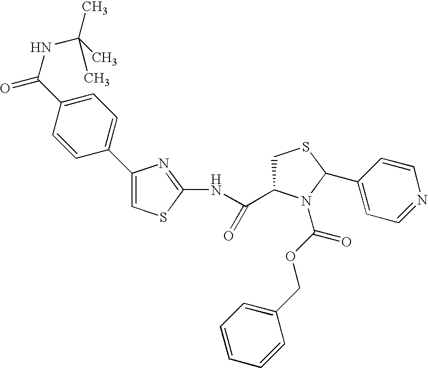 | 4-[4-(4-tert-Butylcarbamoyl-phenyl)-thiazol-2-ylcarbamoyl]-2-pyridin-4-yl-thiazolidine-3-carboxylic acidbenzyl ester |
| 7042 | 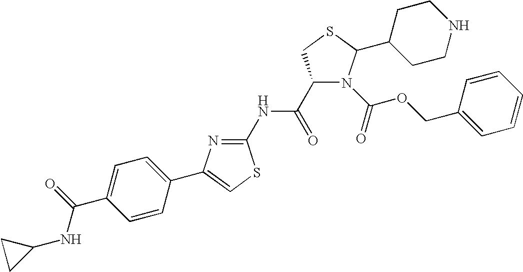 | 4-[4-(4-Cyclopropylcarbamoyl-phenyl)-thiazol-2-ylcarbamoyl]-2-piperidin-4-yl-thiazolidine-3-carboxylic acidbenzyl ester |
| 7043 | 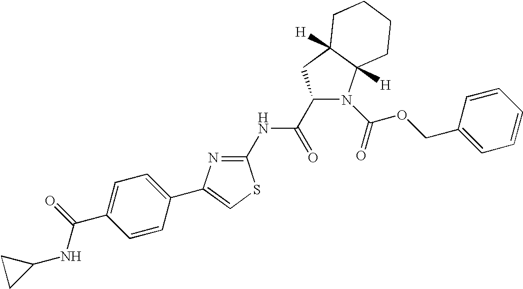 | 2-[4-(4-Cyclopropylcarbamoyl-phenyl)-thiazol-2-ylcarbamoyl]-octahydro-indole-1-carboxylic acid benzylester |
| 7044 | 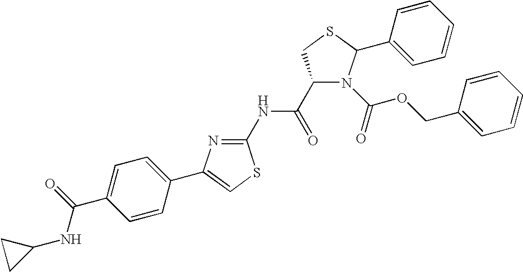 | 4-[4-(4-Cyclopropylcarbamoyl-phenyl)-thiazol-2-ylcarbamoyl]-2-phenyl-thiazolidine-3-carboxylic acidbenzyl ester |
| 7045 | 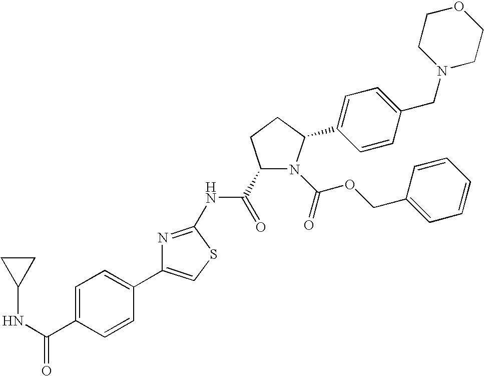 | 2-[4-(4-Cyclopropylcarbamoyl-phenyl)-thiazol-2-ylcarbamoyl]-5-(4-morpholin-4-ylmethyl-phenyl)-pyrrolidine-1-carboxylic acidbenzyl ester |
| 7046 | 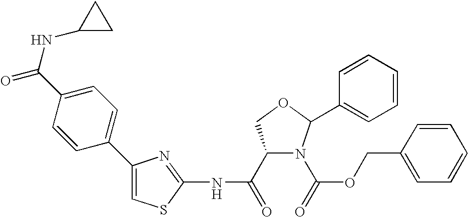 | 4-[4-(4-Cyclopropylcarbamoyl-phenyl)-thiazol-2-ylcarbamoyl]-2-phenyl-oxazolidine-3-carboxylic acidbenzyl ester |
| 7047 | 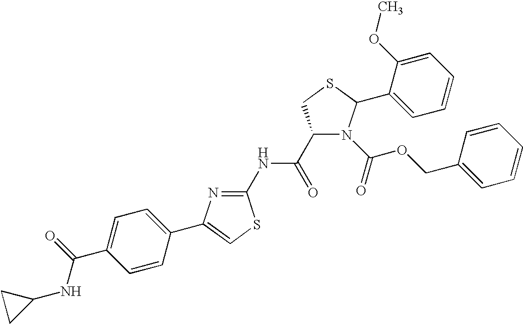 | 4-[4-(4-Cyclopropylcarbamoyl-phenyl)-thiazol-2-ylcarbamoyl]-2-(2-methoxy-phenyl)-thiazolidine-3-carboxylic acid benzyl ester |
| 7048 | 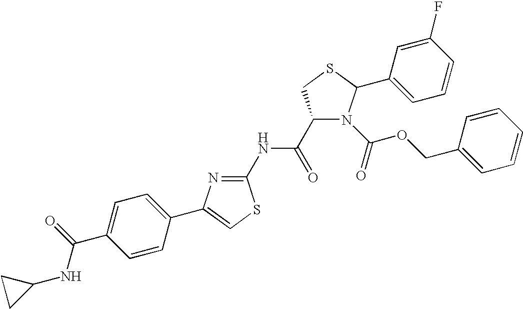 | 4-[4-(4-Cyclopropylcarbamoyl-phenyl)-thiazol-2-ylcarbamoyl]-2-(3-fluoro-phenyl)-thiazolidine-3-carboxylic acid benzyl ester |
| 7049 | 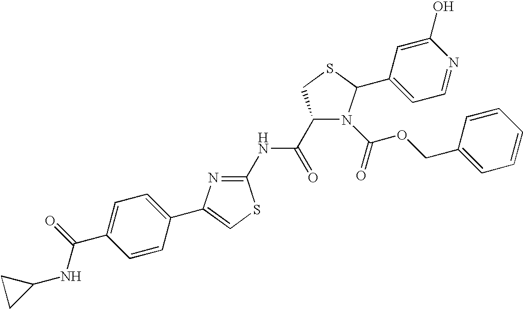 | 4-[4-(4-Cyclopropylcarbamoyl-phenyl)-thiazol-2-ylcarbamoyl]-2-(2-hydroxy-pyridin-4-yl)-thiazolidine-3-carboxylic acid benzyl ester |
| 7050 | 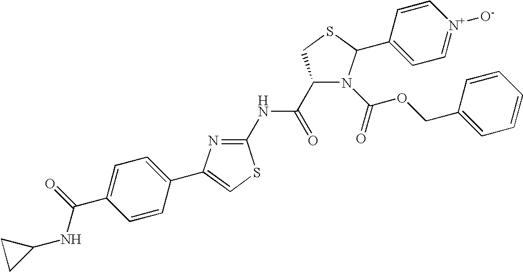 | 4-[4-(4-Cyclopropylcarbamoyl-phenyl)-thiazol-2-ylcarbamoyl]-2-(1-oxy-pyridin-4-yl)-thiazolidine-3-carboxylicacid benzyl ester |
| 7051 | 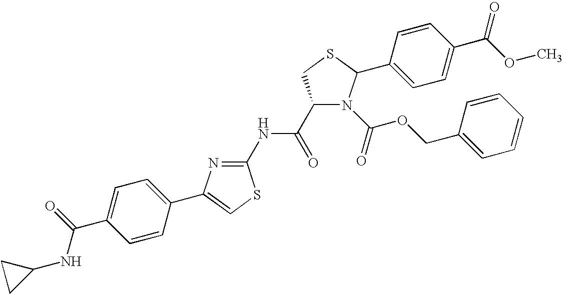 | 4-[4-(4-Cyclopropylcarbamoyl-phenyl)-thiazol-2-ylcarbamoyl]-2-(4-methoxycarbonyl-phenyl)-thiazolidine-3-carboxylic acidbenzyl ester |
| 7052 | 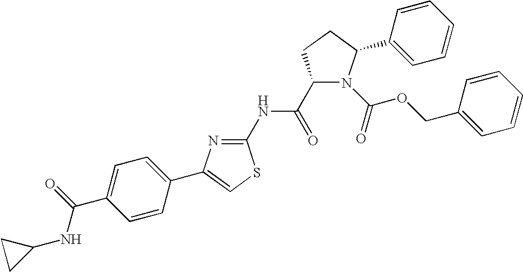 | 2-[4-(4-Cyclopropylcarbamoyl-phenyl)-thiazol-2-ylcarbamoyl]-5-phenyl-pyrrolidine-1-carboxyhc acidbenzyl ester |
| 7053 | 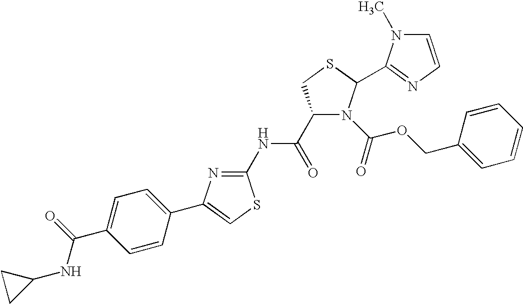 | 4-[4-(4-Cyclopropylcarbamoyl-phenyl)-thiazol-2-ylcarbamoyl]-2-(1-methyl-1H-imidazol-2-yl)-thiazolidine-3-carboxylic acid benzyl ester |
| 7054 | 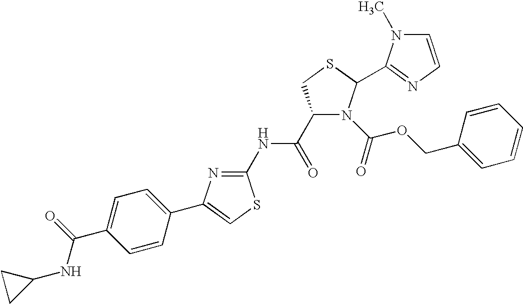 | 4-[4-(4-Cyclopropylcarbamoyl-phenyl)-thiazol-2-ylcarbamoyl]-2-(1-methyl-1H-imidazol-2-yl)-thiazolidine-3-carboxylic acid benzyl ester |
| 7055 | 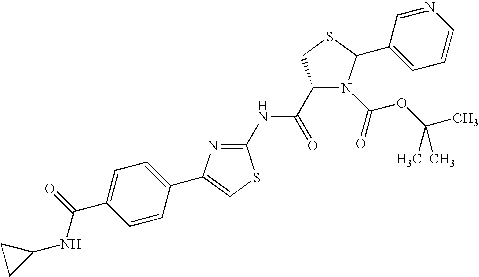 | 4-[4-(4-Cyclopropylcarbamoyl-phenyl)-thiazol-2-ylcarbamoyl]-2-pyridin-3-yl-thiazolidine-3-carboxylic acidtert-butyl ester |
| 7056 | 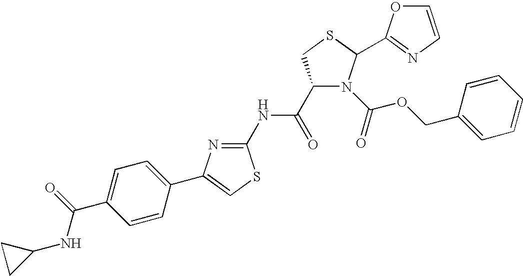 | 4-[4-(4-Cyclopropylcarbamoyl-phenyl)-thiazol-2-ylcarbamoyl]-2-oxazol-2-yl-thiazolidine-3-carboxylic acidbenzyl ester |
| 7057 | 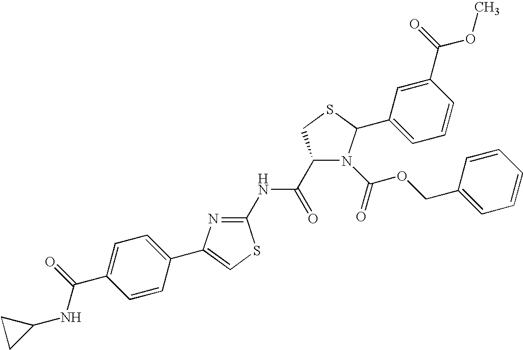 | 4-[4-(4-Cyclopropylcarbamoyl-phenyl)-thiazol-2-ylcarbamoyl]-2-(3-methoxycarbonyl-phenyl)-thiazolidine-3-carboxylic acidbenzyl ester |
| 7058 | 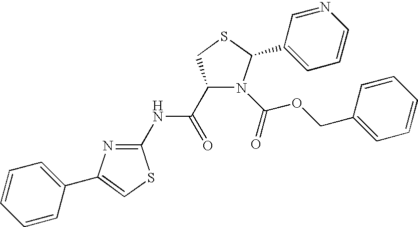 | 4-(4-Phenyl-thiazol-2-ylcarbamoyl)-2-pyridin-3-yl-thiazolidine-3-carboxylic acidbenzyl ester |
| 7059 | 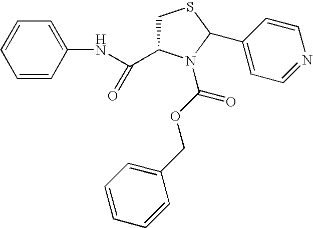 | 4-Phenylcarbamoyl-2-pyridin-4-yl-thiazolidine-3-carboxylicacid benzyl ester |
| 7060 | 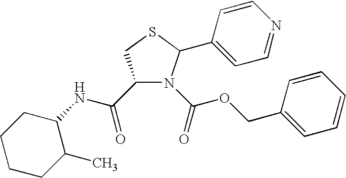 | 4-(2-Methyl-cyclohexylcarbamoyl)-2-pyridin-4-yl-thiazolidine-3-carboxylic acid benzyl ester |
| 7061 | 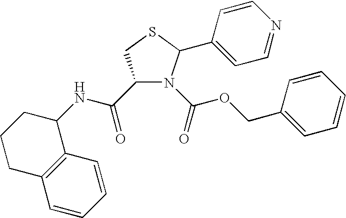 | 2-Pyridin-4-yl-4-(1,2,3,4-tetrahydro-naphthalen-1-ylcarbamoyl)-thiazolidine-3-carboxylic acid benzyl ester |
| 7062 | 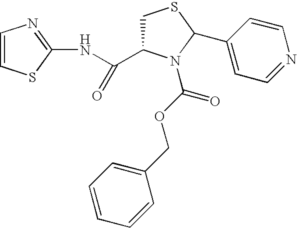 | 2-Pyridin-4-yl-4-(thiazol-2-ylcarbamoyl)-thiazolidine-3-carboxylic acid benzyl ester |
| 7063 | 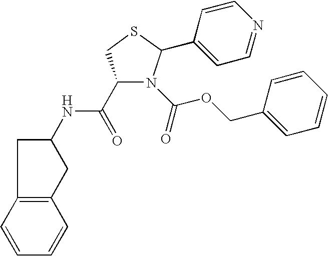 | 4-(Indan-2-ylcarbamoyl)-2-pyridin-4-yl-thiazolidine-3-carboxylic acid benzyl ester |
| 7064 | 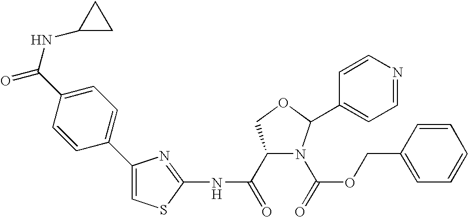 | 4-[4-(4-Cyclopropylcarbamoyl-phenyl)-thiazol-2-ylcarbamoyl]-2-pyridin-4-yl-oxazolidine-3-carboxylic acidbenzyl ester |
| 7065 | 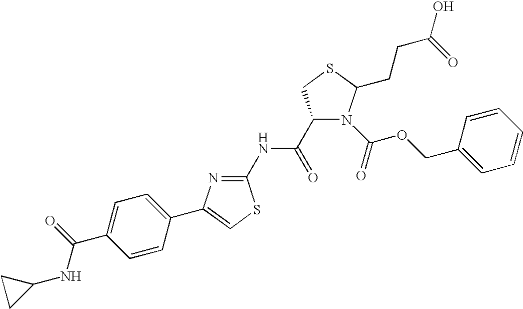 | 2-(2-Carboxy-ethyl)-4-[4-(4-cyclopropylcarbamoyl-phenyl)-thiazol-2-ylcarbamoyl]-thiazolidine-3-carboxylic acidbenzyl ester |
| 7066 | 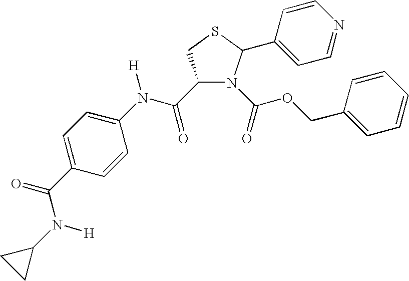 | 4-(4-Cyclopropylcarbamoyl-phenylcarbamoyl)-2-pyridin-4-yl-thiazolidine-3-carboxylicacid benzyl ester |
| 7067 | 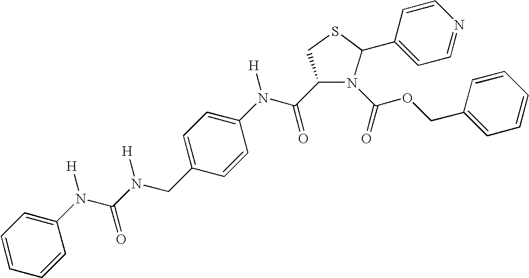 | 4-{4-[(3-Phenyl-ureido)-methyl]-phenylcarbamoyl}-2-pyridin-4-yl-thiazolidine-3-carboxylic acid benzyl ester |
| 7068 | 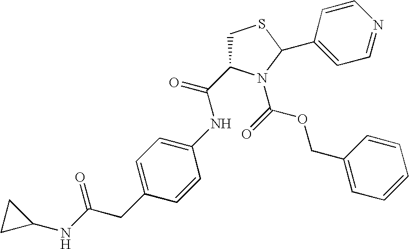 | 4-(4-Cyclopropylcarbamoylmethyl-phenylcarbamoyl)-2-pyridin-4-yl-thiazolidine-3-carboxylicacid benzyl ester |
| 7069 | 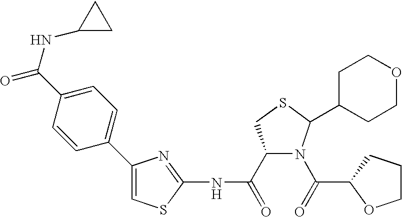 | 3-(Tetrahydro-furan-2-carbonyl)-2-(tetrahydro-pyran-4-yl)-thiazolidine-4-carboxylicacid [4-(4-cyclopropylcarbamoyl-phenyl)-thiazol-2-yl]-amide |
| 7070 | 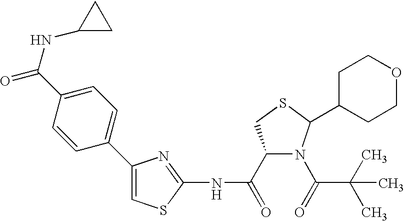 | 3-(2,2-Dimethyl-propionyl)-2-(tetrahydro-pyran-4-yl)-thiazolidine-4-carboxylic acid[4-(4-cyclopropylcarbamoyl-phenyl)-thiazol-2-yl]-amide |
| 7071 | 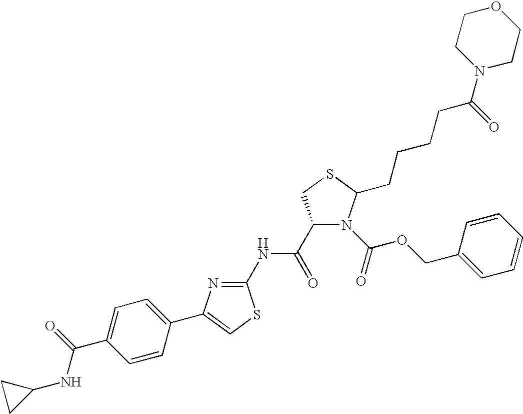 | 4-[4-(4-Cyclopropylcarbamoyl-phenyl)-thiazol-2-ylcarbamoyl]-2-(5-morpholin-4-yl-5-oxo-pentyl)-thiazolidine-3-carboxylic acidbenzyl ester |
| 7072 | 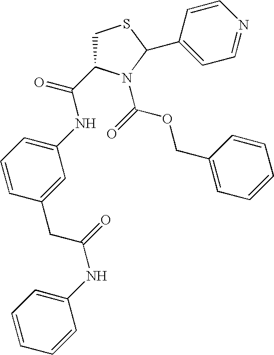 | 4-(3-Phenylcarbamoylmethyl-phenylcarbamoyl)-2-pyridin-4-yl-thiazolidine-3-carboxylicacid benzyl ester |
| 7073 | 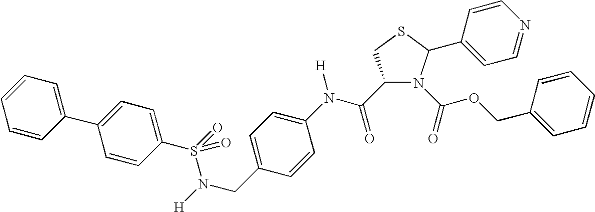 | 4-{4-[(Biphenyl-4-sulfonylamino)-methyl]-phenylcarbamoyl}-2-pyridin-4-yl-thiazolidine-3-carboxylicacid benzyl ester |
| 7074 | 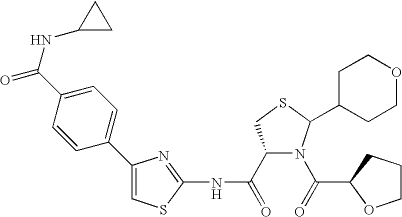 | 3-(Tetrahydro-furan-2-carbonyl)-2-(tetrahydro-pyran-4-yl)-thiazolidine-4-carboxylicacid [4-(4-cyclopropylcarbamoyl-phenyl)-thiazol-2-yl]-amide |
| 7075 | 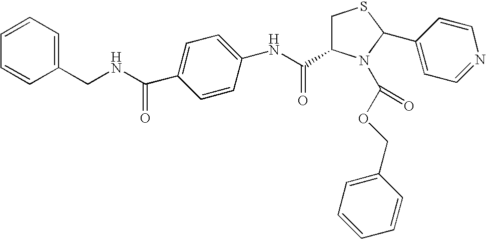 | 4-(4-Benzylcarbamoyl-phenylcarbamoyl)-2-pyridin-4-yl-thiazolidine-3-carboxylicacid benzyl ester |
| 7076 | 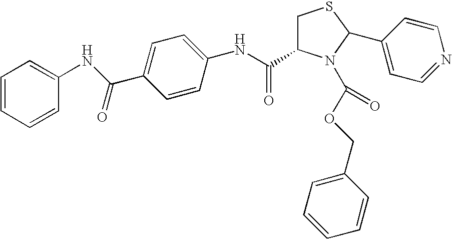 | 4-(4-Phenylcarbamoyl-phenylcarbamoyl)-2-pyridin-4-yl-thiazolidine-3-carboxylicacid benzyl ester |
| 7077 | 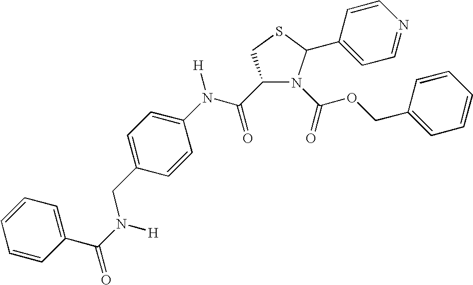 | 4-[4-(Benzoylamino-methyl)-phenylcarbamoyl]-2-pyridin-4-yl-thiazolidine-3-carboxylicacid benzyl ester |
| 7078 | 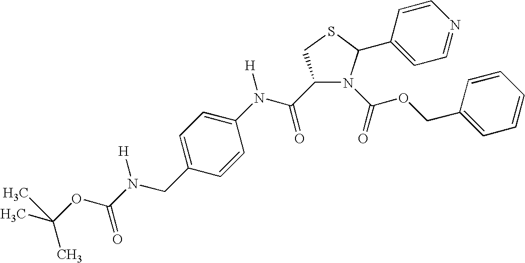 | 4-[4-(tert-Butoxycarbonylamino-methyl)-phenylcarbamoyl]-2-pyridin-4-yl-thiazolidine-3-carboxylicacid benzyl ester |
| 7079 | 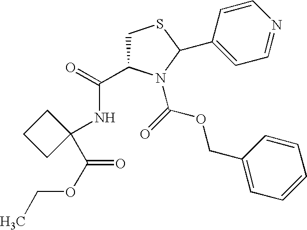 | 4-(1-Ethoxycarbonyl-cyclobutylcarbamoyl)-2-pyridin-4-yl-thiazolidine-3-carboxylic acid benzyl ester |
| 7080 | 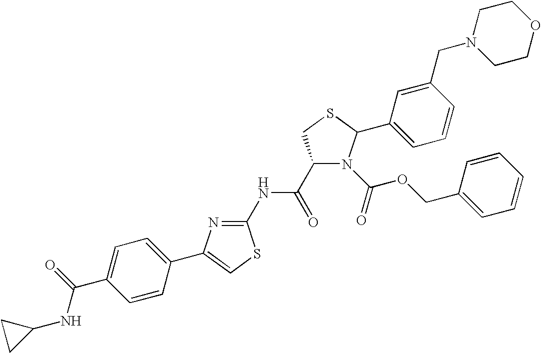 | 4-[4-(4-Cyclopropylcarbamoyl-phenyl)-thiazol-2-ylcarbamoyl]-2-(3-morpholin-4-ylmethyl-phenyl)-thiazolidine-3-carboxylic acidbenzyl ester |
| 7081 | 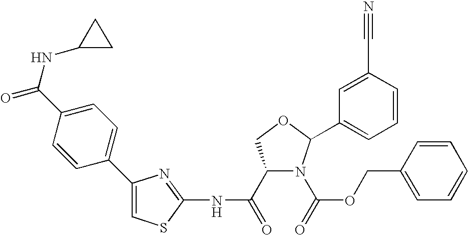 | 2-(3-Cyano-phenyl)-4-[4-(4-cyclopropylcarbamoyl-phenyl)-thiazol-2-ylcarbamoyl]-oxazolidine-3-carboxylic acidbenzyl ester |
| 7082 | 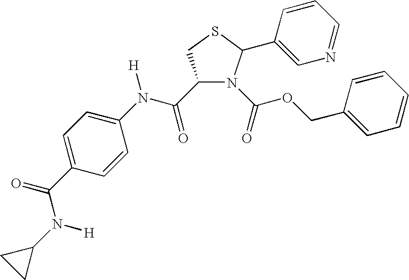 | 4-(4-Cyclopropylcarbamoyl-phenylcarbamoyl)-2-pyridin-3-yl-thiazolidine-3-carboxylicacid benzyl ester |
| 7083 | 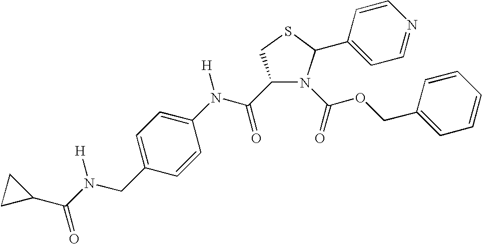 | 4-{4-[(Cyclopropanecarbonyl-amino)-methyl]-phenylcarbamoyl}-2-pyridin-4-yl-thiazolidine-3-carboxylicacid benzyl ester |
| 7084 | 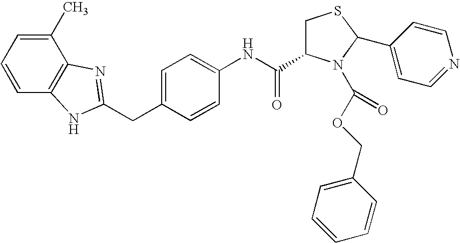 | 4-[4-(4-Methyl-1H-benzoimidazol-2-ylmethyl)-phenylcarbamoyl]-2-pyridin-4-yl-thiazolidine-3-carboxylicacid benzyl ester |
| 7085 | 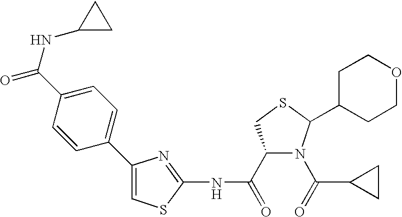 | 3-Cyclopropanecarbonyl-2-(tetrahydro-pyran-4-yl)-thiazolidine-4-carboxylic acid[4-(4-cyclopropylcarbamoyl-phenyl)-thiazol-2-yl]-amide |
| 7086 | 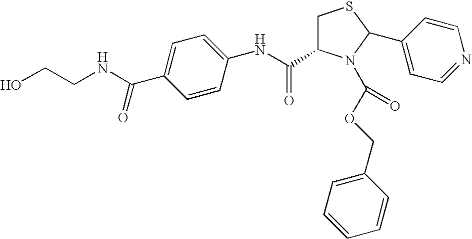 | 4-[4-(2-Hydroxy-ethylcarbamoyl)-phenylcarbamoyl]-2-pyridin-4-yl-thiazolidine-3-carboxylicacid benzyl ester |
| 7087 | 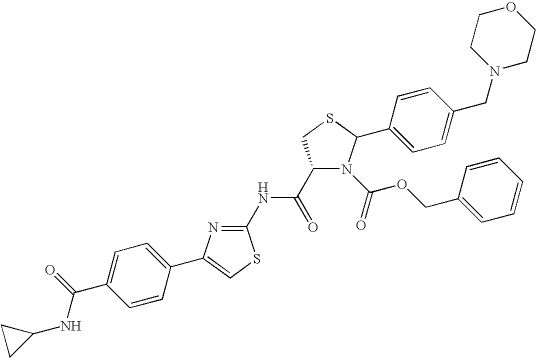 | 4-[4-(4-Cyclopropylcarbamoyl-phenyl)-thiazol-2-ylcarbamoyl]-2-(4-morpholin-4-ylmethyl-phenyl)-thiazolidine-3-carboxylic acidbenzyl ester |
| 7088 | 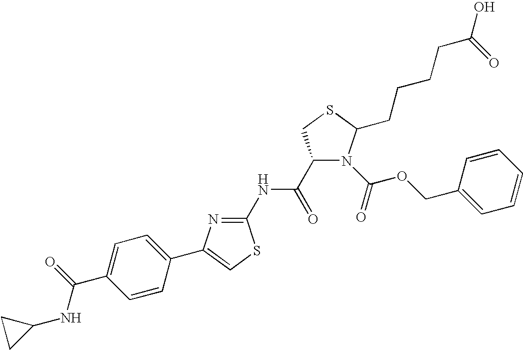 | 2-(4-Carboxy-butyl)-4-[4-(4-cyclopropylcarbamoyl-phenyl)-thiazol-2-ylcarbamoyl]-thiazolidine-3-carboxylic acidbenzyl ester |
| 7089 | 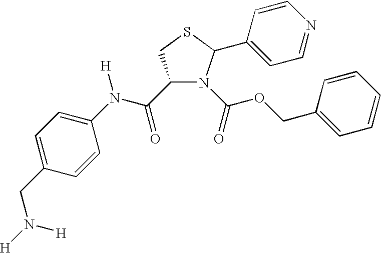 | 4-(4-Aminomethyl-phenylcarbamoyl)-2-pyridin-4-yl-thiazolidine-3-carboxylicacid benzyl ester |
| 7090 | 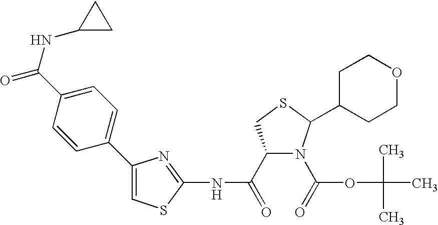 | 4-[4-(4-Cyclopropylcarbamoyl-phenyl)-thiazol-2-ylcarbamoyl]-2-(tetrahydro-pyran-4-yl)-thiazolidine-3-carboxylic acid tert-butyl ester |
| 7091 | 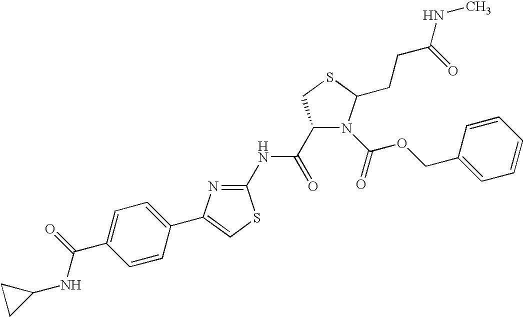 | 4-[4-(4-Cyclopropylcarbamoyl-phenyl)-thiazol-2-ylcarbamoyl]-2-(2-methylcarbamoyl-ethyl)-thiazolidine-3-carboxylic acidbenzyl ester |
| 7092 | 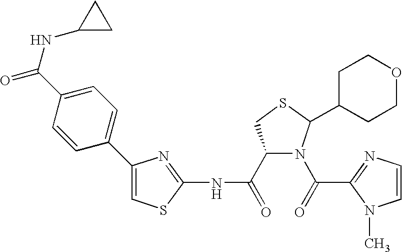 | 3-(1-Methyl-1H-imidazole-2-carbonyl)-2-(tetrahydro-pyran-4-yl)-thiazolidine-4-carboxylicacid [4-(4-cyclopropylcarbamoyl-phenyl)-thiazol-2-yl]-amide |
| 7093 | 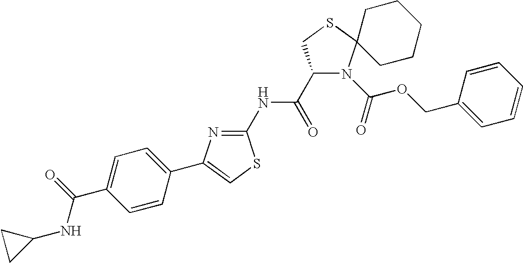 | 3-[4-(4-Cyclopropylcarbamoyl-phenyl)-thiazol-2-ylcarbamoyl]-1-thia-4-aza-spiro[4.5]decane-4-carboxylicacid benzyl ester |
| 7094 | 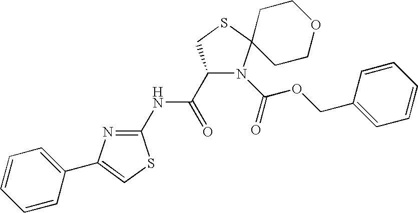 | 3-(4-Phenyl-thiazol-2-ylcarbamoyl)-8-oxa-1-thia-4-aza-spiro[4.5]decane-4-carboxylic acid benzyl ester |
| 7095 | 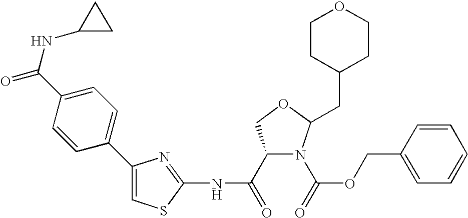 | 4-[4-(4-Cyclopropylcarbamoyl-phenyl)-thiazol-2-ylcarbamoyl]-2-(tetrahydro-pyran-4-ylmethyl)-oxazolidine-3-carboxylic acid benzyl ester |
| 7096 | 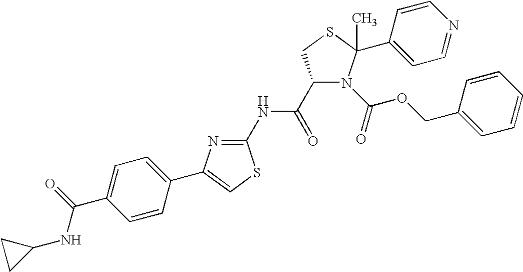 | 4-[4-(4-Cyclopropylcarbamoyl-phenyl)-thiazol-2-ylcarbamoyl]-2-methyl-2-pyridin-4-yl-thiazolidine-3-carboxylic acid benzyl ester |
| 7097 | 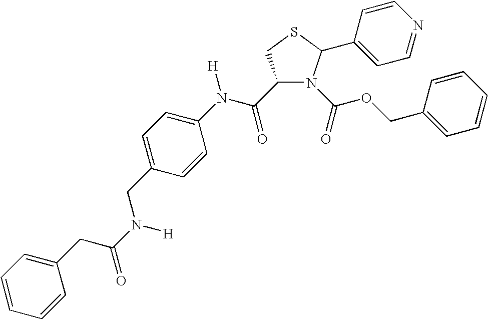 | 4-[4-(Phenylacetylamino-methyl)-phenylcarbamoyl]-2-pyridin-4-yl-thiazolidine-3-carboxylic acid benzyl ester |
| 7098 | 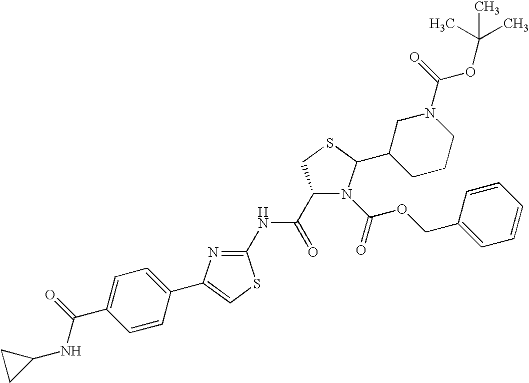 | 3-{3-Benzyloxycarbonyl-4-[4-(4-cyclopropylcarbamoyl-phenyl)-thiazol-2-ylcarbamoyl]-thiazolidin-2-yl}-piperidine-1-carboxylic acidtert-butyl ester |
| 7099 | 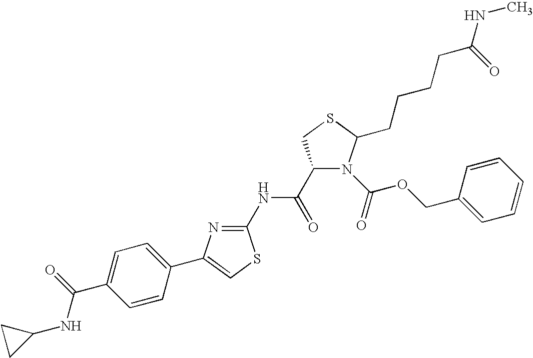 | 4-[4-(4-Cyclopropylcarbamoyl-phenyl)-thiazol-2-ylcarbamoyl]-2-(4-methylcarbamoyl-butyl)-thiazolidine-3-carboxylic acidbenzyl ester |
| 7100 | 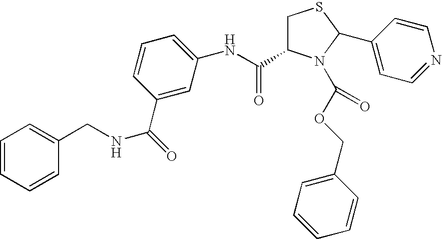 | 4-(3-Benzylcarbamoyl-phenylcarbamoyl)-2-pyridin-4-yl-thiazolidine-3-carboxylicacid benzyl ester |
| 7101 | 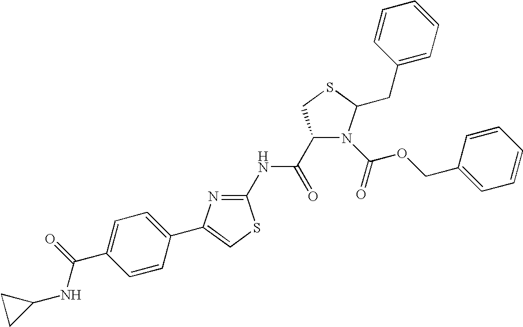 | 2-Benzyl-4-[4-(4-cyclopropylcarbamoyl-phenyl)-thiazol-2-ylcarbamoyl]-thiazolidine-3-carboxylic acidbenzyl ester |
| 7102 | 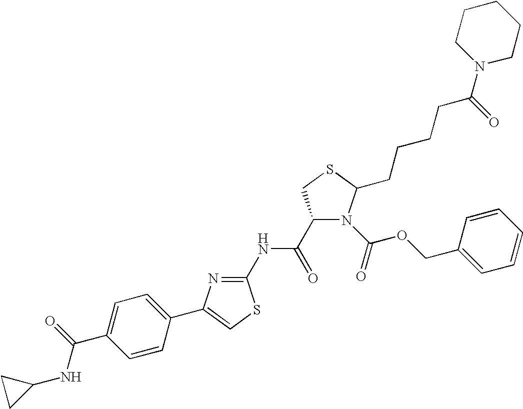 | 4-[4-(4-Cyclopropylcarbamoyl-phenyl)-thiazol-2-ylcarbamoyl]-2-(5-oxo-5-piperidin-1-yl-pentyl)-thiazolidine-3-carboxylic acidbenzyl ester |
| 7103 | 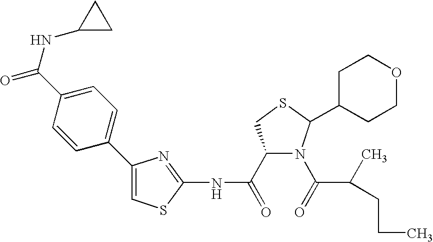 | 3-(2-Methyl-pentanoyl)-2-(tetrahydro-pyran-4-yl)-thiazolidine-4-carboxylic acid[4-(4-cyclopropylcarbamoyl-phenyl)-thiazol-2-yl]-amide |
| 7104 | 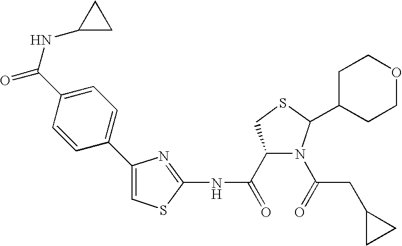 | 3-(2-Cyclopropyl-acetyl)-2-(tetrahydro-pyran-4-yl)-thiazolidine-4-carboxylic acid[4-(4-cyclopropylcarbamoyl-phenyl)-thiazol-2-yl]-amide |
| 7105 | 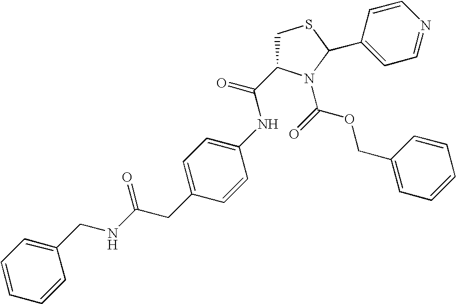 | 4-[4-(Benzylcarbamoyl-methyl)-phenylcarbamoyl]-2-pyridin-4-yl-thiazolidine-3-carboxylic acid benzyl ester |
| 7106 | 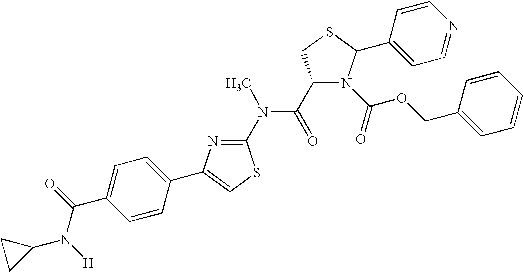 | 4-{[4-(4-Cyclopropylcarbamoyl-phenyl)-thiazol-2-yl]-methyl-carbamoyl}-2-pyridin-4-yl-thiazolidine-3-carboxylic acidbenzyl ester |
| 7107 | 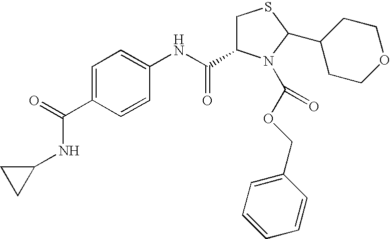 | 4-(4-Cyclopropylcarbamoyl-phenylcarbamoyl)-2-(tetrahydro-pyran-4-yl)-thiazolidine-3-carboxylic acidbenzyl ester |
| 7108 | 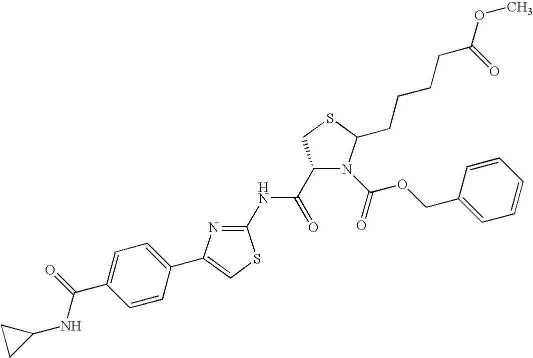 | 4-[4-(4-Cyclopropylcarbamoyl-phenyl)-thiazol-2-ylcarbamoyl]-2-(4-methoxycarbonyl-butyl)-thiazolidine-3-carboxylic acidbenzyl ester |
| 7109 | 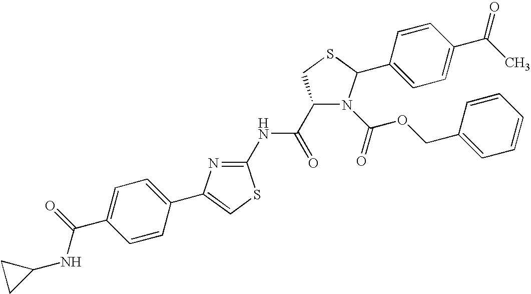 | 2-(4-Acetyl-phenyl)-4-[4-(4-cyclopropylcarbamoyl-phenyl)-thiazol-2-ylcarbamoyl]-thiazolidine-3-carboxylic acidbenzyl ester |
| 7110 | 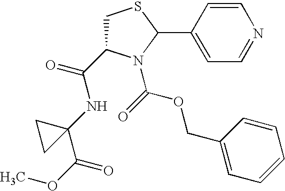 | 4-(1-Methoxycarbonyl-cyclopropylcarbamoyl)-2-pyridin-4-yl-thiazolidine-3-carboxylic acid benzyl ester |
| 7111 | 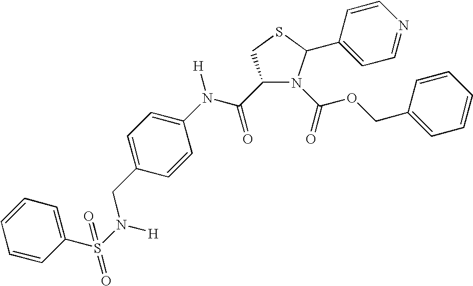 | 4-[4-(Benzenesulfonylamino-methyl)-phenylcarbamoyl]-2-pyridin-4-yl-thiazolidine-3-carboxylic acid benzyl ester |
| 7112 | 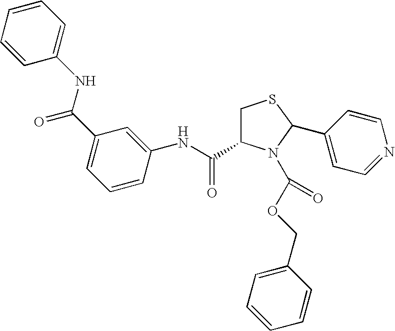 | 4-(3-Phenylcarbamoyl-phenylcarbamoyl)-2-pyridin-4-yl-thiazolidine-3-carboxylicacid benzyl ester |
| 7113 | 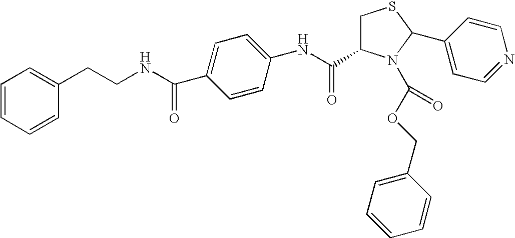 | 4-(4-Phenethylcarbamoyl-phenylcarbamoyl)-2-pyridin-4-yl-thiazolidine-3-carboxylicacid benzyl ester |
| 7114 | 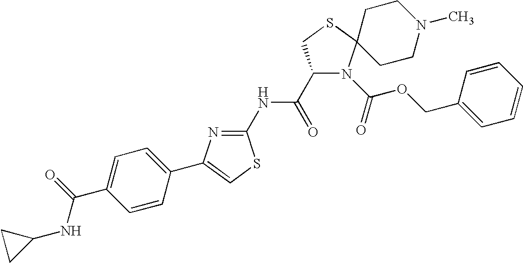 | 3-[4-(4-Cyclopropylcarbamoyl-phenyl)-thiazol-2-ylcarbamoyl]-8-methyl-1-thia-4,8-diaza-spiro[4.5]decane-4-carboxylic acid benzyl ester |
| 7115 | 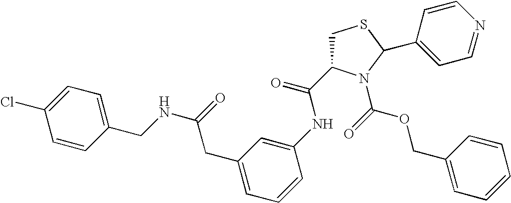 | 4-{3-[(4-Chloro-benzylcarbamoyl)-methyl]-phenylcarbamoyl}-2-pyridin-4-yl-thiazolidine-3-carboxylicacid benzyl ester |
| 7116 | 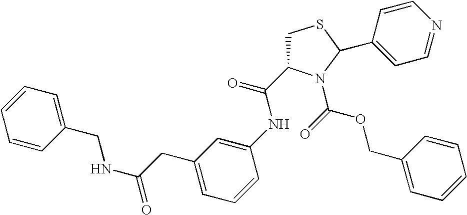 | 4-[3-(Benzylcarbamoyl-methyl)-phenylcarbamoyl]-2-pyridin-4-yl-thiazolidine-3-carboxylic acid benzyl ester |
| 7117 | 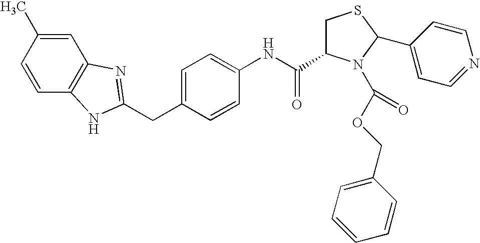 | 4-[4-(5-Methyl-1H-benzoimidazol-2-ylmethyl)-phenylcarbamoyl]-2-pyridin-4-yl-thiazolidine-3-carboxylicacid benzyl ester |
| 7118 | 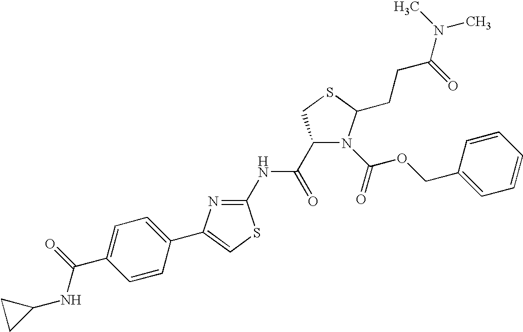 | 4-[4-(4-Cyclopropylcarbamoyl-phenyl)-thiazol-2-ylcarbamoyl]-2-(2-dimethylcarbamoyl-ethyl)-thiazolidine-3-carboxylic acidbenzyl ester |
| 7119 | 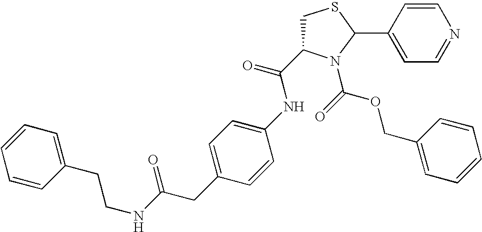 | 4-[4-(Phenethylcarbamoyl-methyl)-phenylcarbamoyl]-2-pyridin-4-yl-thiazolidine-3-carboxylic acid benzyl ester |
| 7120 | 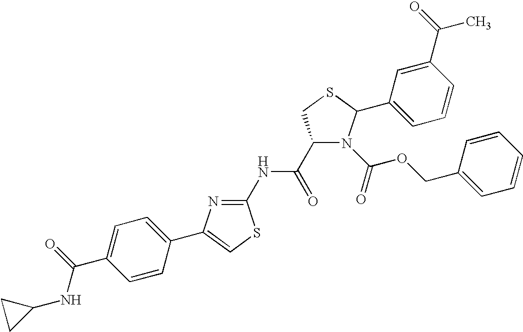 | 2-(3-Acetyl-phenyl)-4-[4-(4-cyclopropylcarbamoyl-phenyl)-thiazol-2-ylcarbamoyl]-thiazolidine-3-carboxylic acidbenzyl ester |
| 7121 | 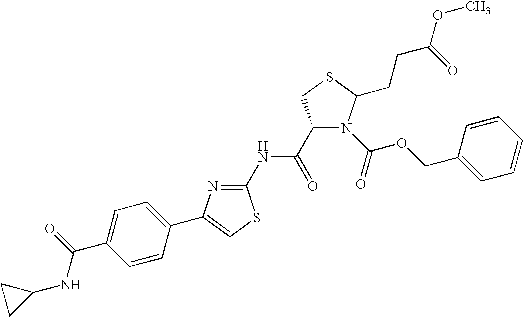 | 4-[4-(4-Cyclopropylcarbamoyl-phenyl)-thiazol-2-ylcarbamoyl]-2-(2-methoxycarbonyl-ethyl)-thiazolidine-3-carboxylic acidbenzyl ester |
| 7122 | 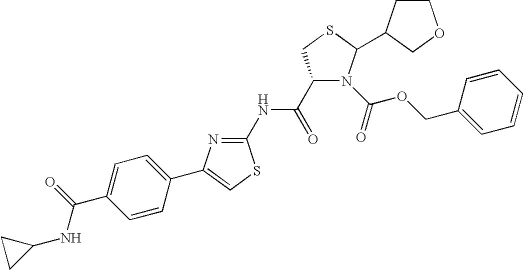 | 4-[4-(4-Cyclopropylcarbamoyl-phenyl)-thiazol-2-ylcarbamoyl]-2-(tetrahydro-furan-3-yl)-thiazolidine-3-carboxylic acid benzyl ester |
| 7123 | 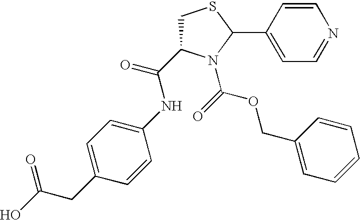 | 4-(4-Carboxymethyl-phenylcarbamoyl)-2-pyridin-4-yl-thiazolidine-3-carboxylicacid benzyl ester |
| 7124 | 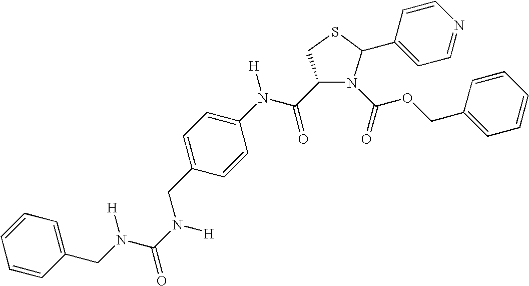 | 4-[4-(3-Benzyl-ureidomethyl)-phenylcarbamoyl]-2-pyridin-4-yl-thiazolidine-3-carboxylicacid benzyl ester |
| 7125 | 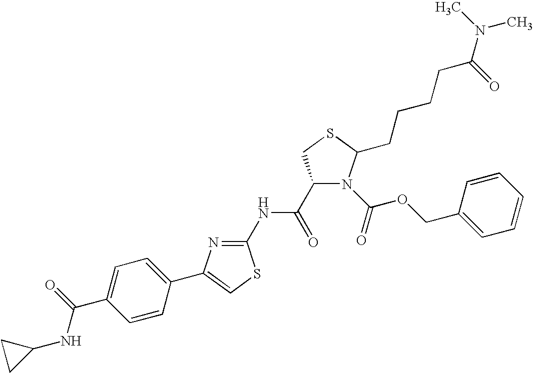 | 4-[4-(4-Cyclopropylcarbamoyl-phenyl)-thiazol-2-ylcarbamoyl]-2-(4-dimethylcarbamoyl-butyl)-thiazolidine-3-carboxylic acidbenzyl ester |
| 7126 | 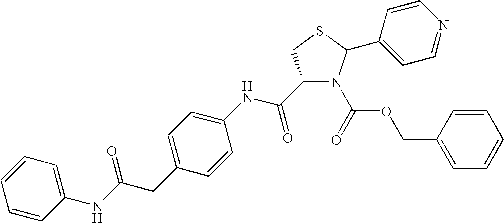 | 4-(4-Phenylcarbamoylmethyl-phenylcarbamoyl)-2-pyridin-4-yl-thiazolidine-3-carboxylicacid benzyl ester |
| 7127 | 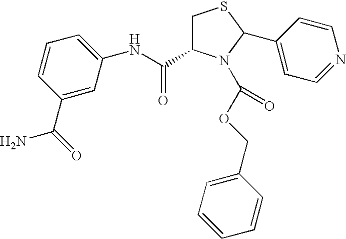 | 4-(3-Carbamoyl-phenylcarbamoyl)-2-pyridin-4-yl-thiazolidine-3-carboxylicacid benzyl ester |
| 7128 | 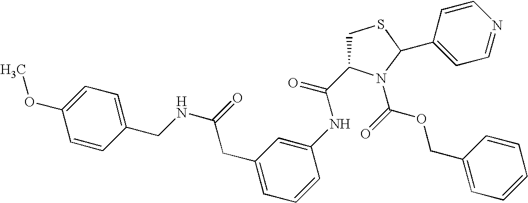 | 4-{3-[(4-Methoxy-benzylcarbamoyl)-methyl]-phenylcarbamoyl}-2-pyridin-4-yl-thiazolidine-3-carboxylicacid benzyl ester |
| 7129 | 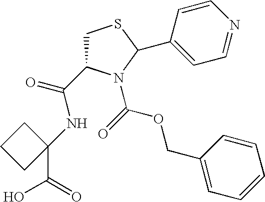 | 4-(1-Carboxy-cyclobutylcarbamoyl)-2-pyridin-4-yl-thiazolidine-3-carboxylic acid benzyl ester |
| 7130 | 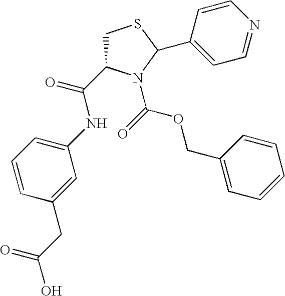 | 4-(3-Carboxymethyl-phenylcarbamoyl)-2-pyridin-4-yl-thiazolidine-3-carboxylicacid benzyl ester |
| 7131 | 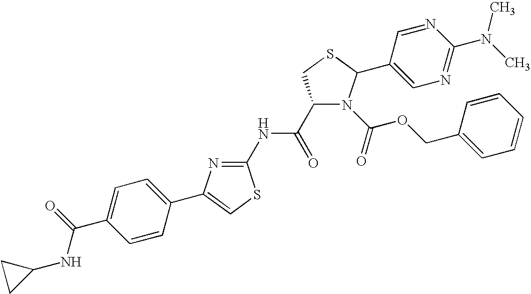 | 4-[4-(4-Cyclopropylcarbamoyl-phenyl)-thiazol-2-ylcarbamoyl]-2-(2-dimethylamino-pyrimidin-5-yl)-thiazolidine-3-carboxylicacid benzyl ester |
| 7132 | 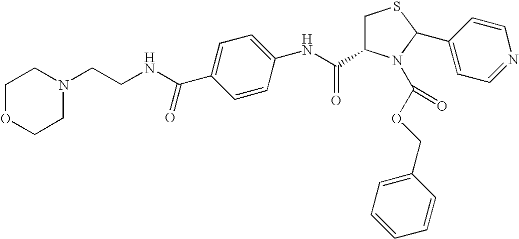 | 4-[4-(2-Morpholin-4-yl-ethylcarbamoyl)-phenylcarbamoyl]-2-pyridin-4-yl-thiazolidine-3-carboxylicacid benzyl ester |
| 7133 | 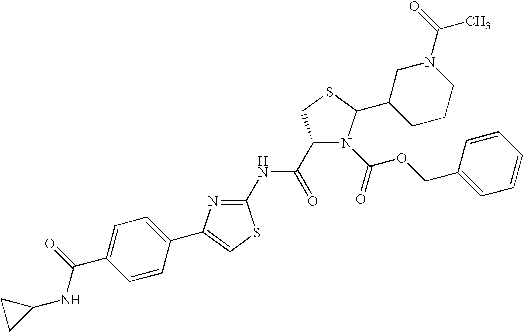 | 2-(1-Acetyl-piperidin-3-yl)-4-[4-(4-cyclopropylcarbamoyl-phenyl)-thiazol-2-ylcarbamoyl]-thiazolidine-3-carboxylic acid benzyl ester |
| 7134 | 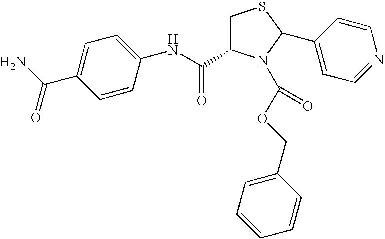 | 4-(4-Carbamoyl-phenylcarbamoyl)-2-pyridin-4-yl-thiazolidine-3-carboxylicacid benzyl ester |
| 7135 | 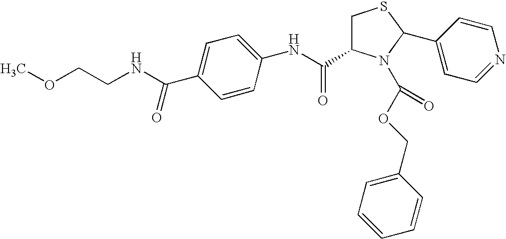 | 4-[4-(2-Methoxy-ethylcarbamoyl)-phenylcarbamoyl]-2-pyridin-4-yl-thiazolidine-3-carboxylicacid benzyl ester |
| 7136 | 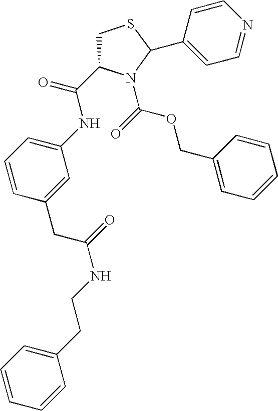 | 4-[3-(Phenethylcarbamoyl-methyl)-phenylcarbamoyl]-2-pyridin-4-yl-thiazolidine-3-carboxylic acid benzyl ester |
| 7137 | 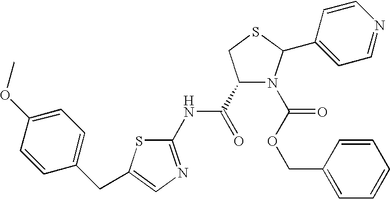 | 4-[5-(4-Methoxy-benzyl)-thiazol-2-ylcarbamoyl]-2-pyridin-4-yl-thiazolidine-3-carboxylic acid benzyl ester |
| 7138 | 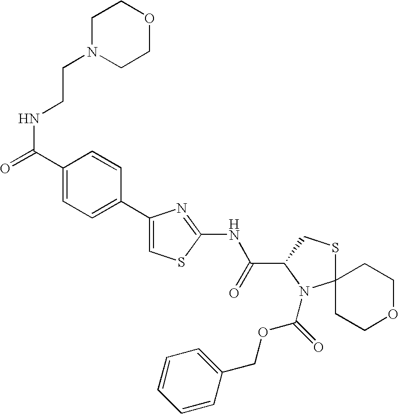 | 3-{4-[4-(2-Morpholin-4-yl-ethylcarbamoyl)-phenyl]-thiazol-2-ylcarbamoyl}-8-oxa-1-thia-4-aza-spiro[4.5]decane-4-carboxylic acid benzyl ester |
| 7139 | 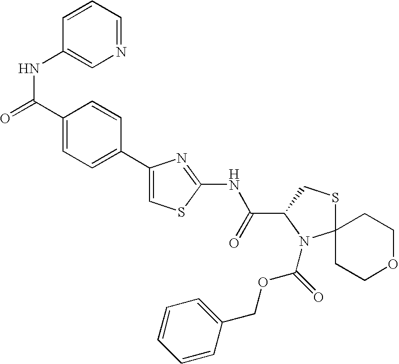 | 3-{4-[4-(Pyridin-3-ylcarbamoyl)-phenyl]-thizol-2-ylcarbamoyl}-8-oxa-1-thia-4-aza-spiro-[4.5]decane-4-carboxylic acid benzyl ester |
| 7140 | 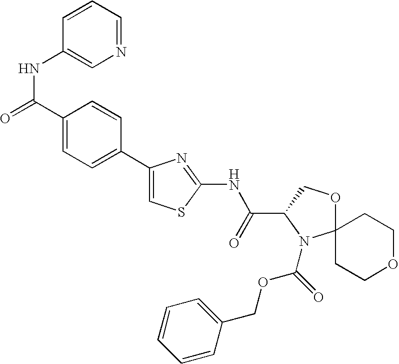 | 3-{4-[4-(Pyridin-3-ylcarbamoyl)-phenyl]-thiazol-2-ylcarbamoyl}-1,8-dioxa-4-aza-spiro[4.5]decane-4-carboxylic acid benzyl ester |
| 7141 | 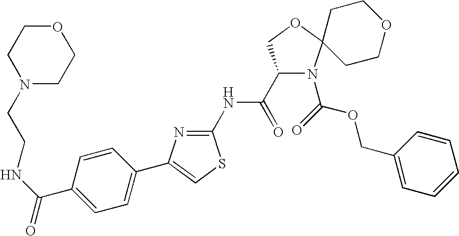 | 3-{4-[4-(2-Morpholin-4-yl-ethylcarbamoyl)-phenyl]-thiazol-2-ylcarbamoyl}-1,8-dioxa-4-aza-spiro[4.5]decane-4-carboxylic acid benzyl ester |

In one embodiment provided is a pharmaceutical composition comprising a pharmaceutically acceptable carrier and a therapeutically effective amount of a compound, stereoisomer, tautomer, pharmaceutically acceptable salt, or prodrug thereof of any one of Formula (I)-(IV) or of the compounds in Table 1. In another embodiment provided is a method for treating a viral infection in a patient mediated at least in part by a virus in the Flaviviridae family of viruses, comprising administering to said patient such compositions. In some aspects, the viral infection is mediated by hepatitis C virus.

In other aspects, the administration of a therapeutically effective amount of the compounds and/or compositions of the invention are used in combination with one or more agents active against hepatitis C virus. These agents include an inhibitor of HCV proteases, HCV polymerase, HCV helicase, HCV NS4B protein, HCV entry, HCV assembly, HCV egress, HCV NS5A protein, or inosine 5′-monophosphate dehydrogenase. In other embodiments, the agent is interferon.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of compounds of Formula (I)-(IV) may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula (I)-(IV) in combination with at least one pharmaceutically acceptable excipient. Acceptable excipients are non-toxic, aid administration, and do not adversely affect the therapeutic benefit of the compound of Formula (I)-(IV). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula (I)-(IV) based on the total formulation, with the balance being one or more suitable pharmaceutical excipients. Preferably, the compound is present at a level of about 1-80 wt %. Representative pharmaceutical formulations containing a compound of Formula (I)-(IV) are described below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV.

References herein to agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharmaceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine(Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-5703 10 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of Formula (I)-(IV) and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of Formula (I)-(IV) and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Accordingly, in one embodiment provided is a method for synthesizing a compound, stereoisomer, tautomer, pharmaceutically acceptable salt, or prodrug of Formula (I)

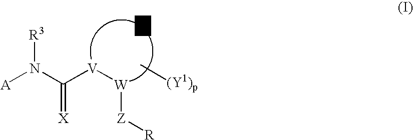

wherein the method comprises
(a) reacting an amine (A)(R³)NH with an acid having the Formula

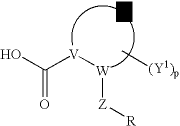

under amide coupling conditions, wherein:
A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
R³ is selected from the group consisting of hydrogen, alkyl, substituted alkyl, cycloalkyl, and substituted cycloalkyl;
X is O or S;
T is C₂-C₆ alkylene or C₁-C₅ heteroalkylene and forms a 4-8 membered ring with V and W;
V and W are both CH, or one of V or W is CH and the other of V or W is N;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl;
(b) optionally reacting the compound of Formula (I) with an acid to form a pharmaceutically acceptable salt thereof, and
(c) optionally converting the compound of Formula (I) to a prodrug thereof.

Suitable amide coupling conditions include the use of coupling reagents. A variety of amide coupling reagents may be used to from the amide bond, including the use of carbodiimides such as N—N′-dicyclohexylcarbodiimide (DCC), N—N′-diisopropylcarbodiimide (DIPCDI), and 1-ethyl-3-(3′-dimethylaminopropyl)carbodiimide (EDCI). The carbodiimides may be used in conjunction with additives such as benzotriazoles 7-aza-1-hydroxybenzotriazole (HOAt), 1-hydroxybenzotriazole (HOBt), and 6-chloro-1-hydroxybenzotriazole (Cl-HOBt).

Amide coupling reagents also include amininum and phosphonium based reagents. Aminium salts include N-[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino)methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP).

The amide formation step may be conducted in a polar solvent such as dimethylformamide (DMF) and may also include an organic base such as diisopropylethylamine (DIPEA).

Scheme 1 shows the synthesis of the compounds of the invention where A is a 4-substituted thiazol-2-yl group, p is 1, and Z-R together form a benzyloxycarbonyl group. Bromide 1.1 is reacted with thiourea to form amine 1.2. The R² group may be further modified at this stage as seen in Example 1. Coupling of amine 1.2 with acid 2.3 using standard amide coupling procedures forms amide 1.3.

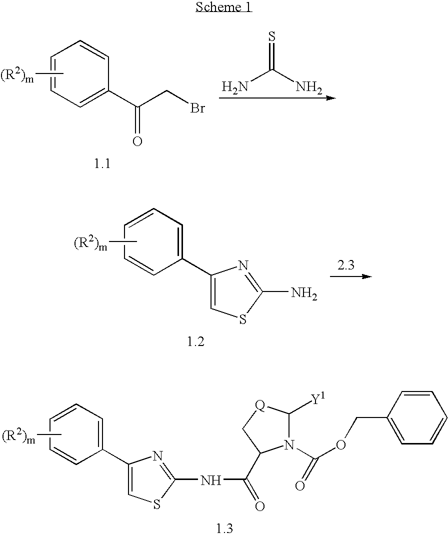

Scheme 2 shows the synthesis of acid 2.3, where for illustrative purposes Q is O or S. Reaction of cysteine or serine 2.1 with aldehyde Y¹CHO or ketone (Y¹)₂CO under cyclizing conditions gives the cyclized derivatives 2.2. Suitable cyclization conditions include use of a base such as potassium acetate in a polar solvent. Amine 2.2 is then reacted with CBZ-Cl (benzyloxycarbonyl chloride) or an equivalent reagent and an organic base such as DIPEA (diisopropylethylamine) in an appropriate solvent such as acetonitrile to give acid 2.3.

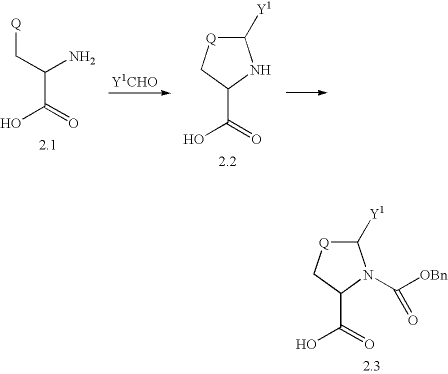

Compound 2.3 where Q is CH₂ may be prepared by using the appropriate substituted pyrrolidine starting material as shown in Scheme 3 or via the procedures shown in Examples 3 and 45. Pyroglutamic acid ethyl ester 3.1 is converted to the t-butoxycarbonyl derivative by treatment with a reagent such as di-tert-butyldicarbonate (BOC)₂O under suitable carbamate protecting group forming conditions. Reaction of 3.2 with a Grignard reagent such as Y¹MgBr gives 3.3. Exposure of 3.3 to an acid such as HCl gives the cyclized imine 3.4 that is then reduced to 3.5 with reducing reagents such as NaBH₄ or with catalytic hydrogenation.

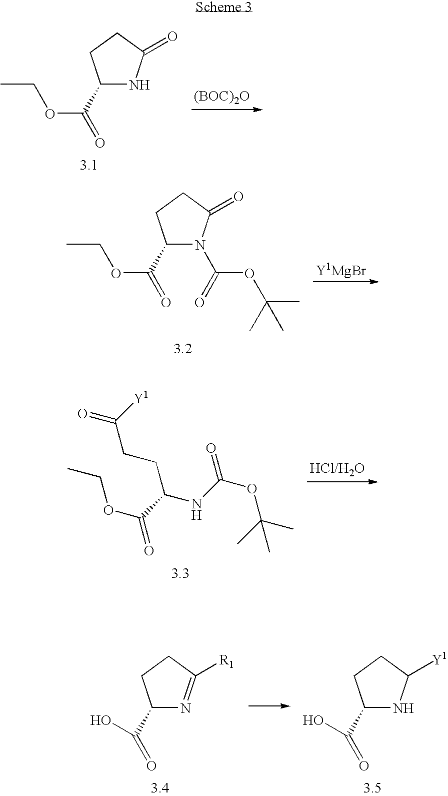

Scheme 4 shows another synthesis of the compounds of the invention where A is a 5-substituted thiazol-2-yl group, V, W, and T together form a (S)-pyrrolidine ring, p is 1, and Z-R together form a benzyloxycarbonyl group. Amine 4.1 is reacted with acid 2.3 to form bromide 4.2. The bromide is next coupled to an aryl boronic acid under Suzuki reaction conditions to form thiazole 4.3. Functionalization of the amino group gives the substituted amines 4.4 and 4.5.

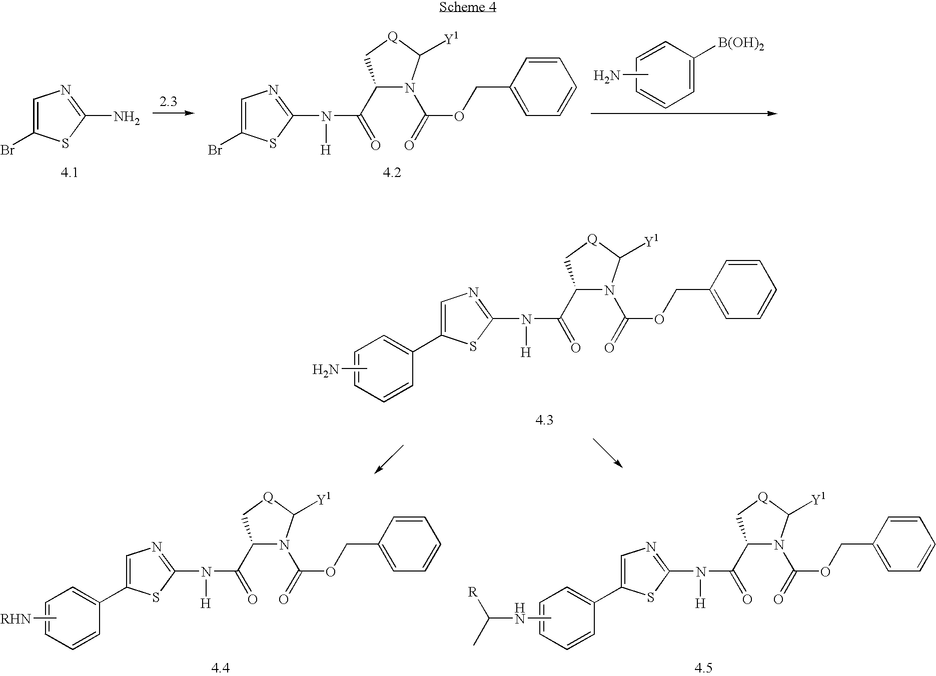

The foregoing and other aspects of the present invention may be better understood in connection with the following representative examples.

### EXAMPLES

The examples below as well as throughout the application, the following abbreviations have the following meanings. If not defined, the terms have their generally accepted meanings.
atm=atmospheres
cm=centimeter
DMF=dimethylformamide
DIPEA=diisopropylethylamine
DIPEA=diisopropylethylamine
DMSO=dimethylsulfoxide
eq.=Equivalents
F.W.=Formula weight
g=gram
HATU=N-[(dimethylamino)-1H-1,2,3-triazolo[4,5-b]pyridine-1-ylmethylene]-N-methylmethanaminium hexafluorophosphate N-oxide
HPLC=high pressure liquid chromatography
KOAc=potassium acetate
L=liter
MeCN=acetonitrile
mg=milligram
mL=milliliter
mmol=millimole
MS=mass spectrum
TEA=triethylamine
TFA=trifluoroacetic acid
THF=tetrahydrofuran
TLC=thin layer chromatography
v/v=volume/volume
μL=microliter

### General Procedure A

The acid (0.2 mmol) was dissolved in DMF (5 mL) and treated with HATU (1.1 eq. 83.6 mg) and DIPEA (2.2 eq, 76 μL) and stirred for 15 minutes. Then 1-[4-(2-amino-thiazol-4-yl)-phenyl]-ethanone (1 eq, 51.8 mg) was added and the mixture stirred at ambient temperature overnight. The reaction was cooled, filtered and the solvents removed. The resulting mixture was redissolved in 5 ml of 90% DMF, 10% water with 0.1% TFA and purified by reverse phase HPLC to give the product.

### Example 1

### 2-[4-(4-Cyclopropylcarbamoyl-phenyl)-thiazol-2-ylcarbamoyl]-indole-1-carboxylic acid benzyl ester (Compound 7001)

### 4-(2-Amino-thiazol-4-yl)-benzoic acid

A solution of 4-acetyl-benzoic acid (10 g, 61 mmol), in HOAc (400 mL) at 55 deg C. was treated with bromine (1 eq., 3.12 mL) dropwise over 10 minutes. After 90 minutes the reaction was cooled, the acetic acid was removed, ethyl acetate (50 mL) was added and then removed to get rid of the remainder of the acetic acid. The crude bromo ketone was then dissolved in ethanol (200 mL) with NaOAc (12 g) and thiourea (1 eq. 4.4 g) was added. The suspension was stirred at room temperature for 15 hours. The solvents were removed and the solids washed with water (3×100 mL) then ether:ethanol (4:1, 3×100 mL) and dried to give the product as a tan solid. MS: 221.2 (M+H⁺).

### 4-(2-Amino-thiazol-4-yl)-N-cyclopropyl-benzamide

4-(2-Amino-thiazol-4-yl)-benzoic acid (4.4 g, 20 mmol) was dissolved in DMF (100 mL) and treated with HATU (1.1 eq. 8.4 g) and DIPEA (2.1 eq, 7.5 mL) and stirred for 15 minutes. Then cyclopropyl amine (1.1 eq, 1.5 mL) was added and the mixture stirred at ambient temperature for 1 hour. The reaction was diluted with 100 mL of water, extracted with EtOAc (3×100 mL) dried with brine (100 mL) and then Na₂SO₄ and the solvents removed. The resulting solid was triturated with ether to give the product >95% purity by HPLC. MS: 260.3 (M+H).

### Indole-1,2-dicarboxylic acid 1-benzyl ester

A solution of 1H-indole-2-carboxylic acid (503.2 mg, 3.1 mmol) in DMF (30 mL) was added to a flame-dried flask and flushed with argon. Then 95% sodium hydride (195.9 mg, 0.78 mmol) was added, and the reaction was stirred at ambient temperature under argon for 5 minutes. Next benzyl chloroformate (1.1 ml, 0.77 mmol) was slowly added, and the reaction was stirred at ambient temperature under argon for 30 minutes. The reaction was filtered and purified by reverse phase HPLC. A mixture of desired product and benzyl alcohol was isolated. The mixture was dissolved in ether and treated with aqueous sodium carbonate. The aqueous layer was isolated, acidified with 1M HCl, and extracted with ether. The organic layer was isolated and dried with anhydrous magnesium sulfate, and the solvent was removed to give the desired product.

### 2-[4-(4-Cyclopropylcarbamoyl-phenyl)-thiazol-2-ylcarbamoyl]-indole-1-carboxylic acid benzyl ester (Compound 7001)

Indole-1,2-dicarboxylic acid 1-benzyl ester (45.5 mg, 0.15 mmol) and HATU (58.6 mg, 0.15 mmol) were dissolved in DMF (2 mL). DIPEA (53.6 μL, 0.31 mmol) was then added, and the reaction was stirred at ambient temperature for 15 minutes. Then 4-(2-amino-thiazol-4-yl)-N-cyclopropyl-benzamide (40.1 mg, 0.15 mmol) was added. After stirring at ambient temperature for approximately 5 hours, the reaction was stirred at 50° C. overnight. The reaction was cooled, filtered, and purified by reverse phase HPLC to give the desired product. Yield 10.4 mg. MS: 537.2 (M+H⁺); H¹ NMR (DMSO-d₆): δ (ppm) 0.55-0.74 (m, 4H), 2.78-2.91 (m, 1H), 5.34-5.45 (s, 2H), 7.14-7.51 (m, 9H), 7.69-7.79 (m, 6H), 8.41-8.47 (m, 1H), 12.95-13.05 (s, 1H).

### Example 2

### (S)-3-[4-(4-Cyclopropylcarbamoyl-phenyl)-thiazol-2-ylcarbamoyl]-3,4-dihydro-1H-isoquinoline-2-carboxylic acid benzyl ester (Compound 7002)

From (S)-3,4-Dihydro-1H-isoquinoline-2,3-dicarboxylic acid 2-benzyl ester following General Procedure A. Yield 15 mg. MS: 553.3 (M+H). H¹-NMR (DMSO-d₆): δ (ppm) 12.5 (s, 1H), 8.4 (m, 1H), 7.9-7.7 (m, 5H), 7.4-7.1 (m, 1H), 5.2-4.5 (m, 6H), 3.3-3.1 (m, 2H), 2.8 (m, 1H), 0.7-0.5 (m, 4H).

### Example 3

### (2S,5R)-2-[4-(4-Cyclopropylcarbamoyl-phenyl)-thiazol-2-ylcarbamoyl]-5-pyridin-4-yl-pyrrolidine-1-carboxylic acid benzyl ester (Compound 7003)

### (S)-2-tert-Butoxycarbonylamino-4-(pyridine-4-carbonyl)-pentanedioic acid 5-ethyl ester 1-methyl ester

(S)-2-tert-Butoxycarbonylamino-3-iodo-propionic acid methyl ester (550 mg, 1.67 mmol) was dissolved in DMF (5 mL) and treated with Cs₂CO₃ (1.1 g) and 3-Oxo-3-pyridin-4-yl-propionic acid ethyl ester (1 eq., 322 mg) and heated to 60° C. and stirred overnight. The solution was then cooled, TFA was added to adjust the pH to 1 and purified by reverse phase HPLC to give the product. MS: 395 (M+H⁺).

### (S)-5-Pyridin-4-yl-3,4-dihydro-2H-pyrrole-2-carboxylic acid

(S)-2-tert-Butoxycarbonylamino-4-(pyridine-4-carbonyl)-pentanedioic acid 5-ethyl ester 1-methyl ester (20 mg) was dissolved in 6 M HCl (10 mL) and heated to 45 C overnight. The solution was then cooled and the solvents removed under vacuum. The crude product was redissolved in 5 ml of 90% DMF, 10% water with 0.1% TFA and purified by reverse phase HPLC to give the product. MS: 191 (M+H⁺).

### (2S,5R)-5-Pyridin-4-yl-pyrrolidine-2-carboxylic acid

(2S,5R)-5-Pyridin-4-yl-3,4-dihydro-2H-pyrrole-2-carboxylic acid (171 mg) was dissolved in ethanol (50 mL) with platinum oxide (2 mg) and hydrogenated at 30 psi for 2 hours. The solution was filtered, the solvents removed and used directly in the next reaction. MS: 193 (M+H⁺).

### (2S,5R)-5-Pyridin-4-yl-pyrrolidine-1,2-dicarboxylic acid 1-benzyl ester

(2S,5R)-5-Pyridin-4-yl-pyrrolidine-2-carboxylic acid (175 mg, 0.9 mmol) was dissolved in dichloromethane (4 mL) and treated with triethylamine (1 mL), then carbonic acid benzyl ester 2,5-dioxo-pyrrolidin-1-yl ester (1.3 mmol) and DMAP (10 mg). The solution was then stirred at room temperature for 90 minutes. The solvents were removed and the crude product was redissolved in 5 ml of 90% DMF, 10% water with 0.1% TFA and purified by reverse phase HPLC to give the product. MS: 327 (M+H⁺).

### (2S,5R)-2-[4-(4-Cyclopropylcarbamoyl-phenyl)-thiazol-2-ylcarbamoyl]-5-pyridin-4-yl-pyrrolidine-1-carboxylic acid benzyl ester (Compound 7003)

A solution of (2S,5R)-5-Pyridin-4-yl-pyrrolidine-1,2-dicarboxylic acid 1-benzyl ester (150 mg, 0.3 mmol) in DMF (5 mL) was treated with HATU (1.1 eq. 150 mg) and DIPEA (2.2 eq, 150 μL) and stirred for 15 minutes. Then 1-[4-(2-Amino-thiazol-4-yl)-phenyl]-ethanone (1 eq, 100 mg) was added and the mixture stirred at 45° C. overnight. The reaction was cooled, filtered and the solvents removed. The resulting mixture was redissolved in 5 ml of 90% DMF, 10% water with 0.1% TFA and purified by reverse phase HPLC to give the product. Yield 17.3 mg. MS: 568 (M+H⁺); H¹-NMR (DMSO-d₆): δ (ppm) 12.2 (s, 1H), 8.8 (m, 2H), 8.4 (m, 1H), 8.2 (m, 1H), 7.9-7.8 (m, 5H), 7.2-6.9 (m, 5H), 4.7 (m, 4H), 2.8 (m, 1H), 2.5 (m, 1H), 1.8 (m, 2H), 0.7-0.6 (m, 4H).

### Example 4

### (R)-4-[4-(4-Cyclopropylcarbamoyl-phenyl)-thiazol-2-ylcarbamoyl]-2-(tetrahydro-pyran-4-ylmethyl)-thiazolidine-3-carboxylic acid benzyl ester (compound 7004)

### (R)-2-(Tetrahydro-pyran-4-ylmethyl)-thiazolidine-4-carboxylic acid

To a solution of L-cysteine hydrochloride (400 mg, 2.54 mmol) in distilled water (4 mL), potassium acetate (275 mg, 2.80 mmol) was added. Once the solids went into solution, methanol (4 mL) was added followed by (tetrahydro-pyran-4-yl)-acetaldehyde (390 mg, 3.04 mmol). A precipitate crashed out of solution within 30 minutes. The solvent was removed to give the crude product. No further purification steps were taken.

### (R)-2-(Tetrahydro-pyran-4-ylmethyl)-thiazolidine-3,4-dicarboxylic acid 3-benzyl ester

To a solution of phenyl-methanol (128 μL, 1.24 mmol) in acetonitrile (3 mL), triethylamine (344 μL, 2.47 mmol) was added followed by carbonic acid bis-(2,5-dioxo-pyrrolidin-1-yl) ester (316 mg, 1.23 mmol). The reaction was stirred at ambient temperature for one hour. The solvent was removed, and the residue was redissolved in dry dichloromethane (3 mL). Then triethylamine (172 μL, 1.23 mmol), DMAP (5 mg), and (R)-2-(Tetrahydro-pyran-4-ylmethyl)-thiazolidine-4-carboxylic acid (285 mg, 1.23 mmol) were added. The reaction was stirred at ambient temperature for 4 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(tetrahydro-pyran-4-ylmethyl)-thiazolidine-3-carboxylic acid benzyl ester (compound 7004)

(R)-2-(Tetrahydro-pyran-4-ylmethyl)-thiazolidine-3,4-dicarboxylic acid 3-benzyl ester (89.3 mg, 0.24 mmol) was dissolved in DMF (1.5 mL). DIPEA (85 μL, 0.49 mmol) was added, followed by HATU (93 mg, 0.24 mmol). The reaction was stirred at ambient temperature for 15 minutes. Then 4-(2-amino-thiazol-4-yl)-N-cyclopropyl-benzamide (Example 1, 63.4 mg, 0.24 mmol) was added, and the reaction was stirred at ambient temperature for 3 hours. The mixture was then heated to 50° C. overnight. The reaction was cooled, filtered, and purified by reverse phase HPLC to give a diastereomeric mixture of the desired product. Yield 33.1 mg. MS: 507.3 (M+H⁺); H¹ NMR (DMSO-d₆): δ (ppm) 0.52-0.78 (m, 4H), 0.95-1.34 (m, 3H), 1.46-2.00 (m, 5H), 2.80-2.92 (m, 1H), 3.04-3.38 (m, 3H), 3.65-3.90 (m, 2H), 4.78-5.33 (m, 4H), 7.02-7.47 (m, 5H), 7.78-8.03 (m, 5H), 8.42-8.50 (d, 1H).

### Example 5

### (2S,5R)-2-[4-(4-Cyclopropylcarbamoyl-phenyl)-thiazol-2-ylcarbamoyl]-5-phenyl-pyrrolidine-1-carboxylic acid tert-butyl ester (compound 7005)

(2S,5R)-5-Phenyl-pyrrolidine-1,2-dicarboxylic acid 1-tert-butyl ester (55.1 mg, 0.19 mmol) was dissolved in DMF (2 mL). DIPEA (66 μL, 0.38 mmol) was added followed by HATU (72.5 mg, 0.19 mmol). The reaction was stirred at ambient temperature for 15 minutes. Then 4-(2-amino-thiazol-4-yl)-N-cyclopropyl-benzamide (Example 1, 47.6 mg, 0.18 mmol) was added, and the reaction was stirred at ambient temperature for 6 hours. The reaction was then heated to 45° C. overnight. The reaction was cooled, filtered, and purified by reverse phase HPLC to give the desired product. Yield 5.0 mg. MS: 533.2 (M+H⁺); H¹ NMR (DMSO-d₆): δ (ppm) 0.58-0.77 (m, 4H), 1.02-1.10 (s, 4H), 1.21-1.28 (s, 5H), 2.50-2.70 (m, 3H), 7.16-7.38 (m, 3H), 7.59-7.66 (m, 2H), 7.79-8.01 (m, 5H), 8.41-8.47 (m, 1H).

### Example 6

### (2S,4R)-4-Benzyl-2-[4-(4-cyclopropylcarbamoyl-phenyl)-thiazol-2-ylcarbamoyl]-pyrrolidine-1-carboxylic acid benzyl ester (Compound 7006)

### (2S,4R)-4-Benzyl-pyrrolidine-1,2-dicarboxylic acid 1-benzyl ester

(2S,4R)-4-Benzyl-pyrrolidine-2-carboxylic acid (249.5 mg, 1.22 mmol) was dissolved in DMF (10 mL) and distilled water (2 mL). The solution was cooled to 0° C., and DIPEA (530 μL, 3.04 mmol) was added followed by benzyl chloroformate (260 μL, 1.82 mmol). The reaction was stirred at 0° C. and allowed to warm to ambient temperature and stirred overnight. The reaction was then filtered and purified by reverse phase HPLC to give the desired product.

### (2S,4R)-4-Benzyl-2-[4-(4-cyclopropylcarbamoyl-phenyl)-thiazol-2-ylcarbamoyl]-pyrrolidine-1-carboxylic acid benzyl ester (Compound 7006)

(2S,4R)-4-Benzyl-pyrrolidine-1,2-dicarboxylic acid 1-benzyl ester (59.4 mg, 0.18 mmol) was dissolved in DMF (1.5 mL). DIPEA (60 μL, 0.34 mmol) was added followed by HATU (66.1 mg, 0.17 mmol). The reaction was stirred at ambient temperature for 15 minutes. Then 4-(2-Amino-thiazol-4-yl)-N-cyclopropyl-benzamide (Example 1, 45.2 mg, 0.17 mmol) was added, and the reaction was stirred at ambient temperature for 8 hours. The reaction was then heated to 50° C. overnight. The reaction was cooled, filtered, and purified by reverse phase HPLC to give the desired product. Yield 26.1 mg. MS: 581.2 (M+H⁺); H¹ NMR (DMSO-d₆): δ (ppm) 0.54-0.74 (m, 4H), 2.54-2.91 (m, 2H), 3.09-3.20 (m, 1H), 3.45-3.90 (m, 4H), 4.52-4.65 (m, 1H), 4.87-5.14 (m, 2H), 7.02-7.40 (m, 10H), 7.75-7.97 (m, 5H), 8.40-8.46 (m, 1H), 12.40-12.50 (m, 1H).

### Example 7

### (R)-4-[4-(4-Cyclopropylcarbamoyl-phenyl)-thiazol-2-ylcarbamoyl]-2-quinolin-4-yl-thiazolidine-3-carboxylic acid benzyl ester (Compound 7007)

### (R)-2-Quinolin-4-yl-thiazolidine-4-carboxylic acid

To a solution of L-cysteine hydrochloride (400 mg, 2.54 mmol) in distilled water (4 mL), potassium acetate (275 mg, 2.80 mmol) was added. Once the solids went into solution, methanol (4 mL) was added followed by quinoline-4-carbaldehyde (478.2 mg, 3.04 mmol). Precipitate crashed out of solution within one hour, and the reaction was stirred at ambient temperature overnight. The solvent was removed, and no further purification steps were taken.

### (R)-2-Quinolin-4-yl-thiazolidine-3,4-dicarboxylic acid 3-benzyl ester

(R)-2-Quinolin-4-yl-thiazolidine-4-carboxylic (661 mg, 2.54 mmol) was dissolved in DMF (12 mL). The solution was cooled to 0° C., and DIPEA (665 μL, 3.82 mmol) was added followed by benzyl chloroformate (905 μL, 6.34 mmol). The reaction was stirred at 0° C. and allowed to warm to ambient temperature over 3 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-quinolin-4-yl-thiazolidine-3-carboxylic acid benzyl ester (Compound 7007)

(R)-2-Quinolin-4-yl-thiazolidine-3,4-dicarboxylic acid 3-benzyl ester (95.1 mg, 0.24 mmol) was dissolved in DMF (1.5 mL). DIPEA (82 μL, 0.47 mmol) was added followed by HATU (89.8 mg, 0.24 mmol). The reaction was stirred at ambient temperature for 15 minutes. Then 4-(2-amino-thiazol-4-yl)-N-cyclopropyl-benzamide (Example 1, 61.6 mg, 0.24 mmol) was added, and the reaction was stirred at ambient temperature for 3 hours. The reaction was then heated to 50° C. overnight. The reaction was cooled, filtered, and purified by reverse phase HPLC to give a diastereomeric mixture of the desired product. Yield 6.8 mg. MS: 636.2 (M+H⁺); H¹ NMR (DMSO-d₆): δ (ppm) 0.45-0.80 (m, 4H), 4.86-5.20 (m, 3H), 6.84-7.52 (m, 7H), 7.66-8.33 (m, 8H), 8.41-8.63 (m, 2H).

### Example 8

### (R)-4-[4-(4-Cyclopropylcarbamoyl-phenyl)-thiazol-2-ylcarbamoyl]-2-(3-fluoro-pyridin-4-yl)-thiazolidine-3-carboxylic acid benzyl ester (Compound 7008)

### (R)-2-(3-Fluoro-pyridin-4-yl)-thiazolidine-4-carboxylic acid

To a solution of L-cysteine hydrochloride (400 mg, 2.54 mmol) in distilled water (4 mL), potassium acetate (275 mg, 2.80 mmol) was added. Once the solids went into solution, methanol (4 mL) was added followed by 3-fluoro-pyridine-4-carbaldehyde (305 μL, 3.06 mmol). Precipitate crashed out of solution within one hour, and the reaction was stirred at ambient temperature overnight. The solvent was removed, and no further purification steps were taken.

### (R)-2-(3-Fluoro-pyridin-4-yl)-thiazolidine-3,4-dicarboxylic acid 3-benzyl ester

(R)-2-(3-Fluoro-pyridin-4-yl)-thiazolidine-4-carboxylic acid (579 mg, 2.54 mmol) was dissolved in DMF (12 mL). The solution was cooled to 0° C., and DIPEA (665 μL, 3.82 mmol) was added followed by benzyl chloroformate (905 μL, 6.34 mmol). The reaction was stirred at 0° C. and allowed to warm to ambient temperature over 3 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(3-fluoro-pyridin-4-yl)-thiazolidine-3-carboxylic acid benzyl ester (Compound 7008)

(R)-2-(3-Fluoro-pyridin-4-yl)-thiazolidine-3,4-dicarboxylic acid 3-benzyl (93.0 mg, 0.26 mmol) was dissolved in DMF (1.5 mL). DIPEA (87 μL, 0.50 mmol) was added followed by HATU (94.7 mg, 0.25 mmol). The reaction was stirred at ambient temperature for 15 minutes. Then 4-(2-amino-thiazol-4-yl)-N-cyclopropyl-benzamide (Example 1, 64.7 mg, 0.25 mmol) was added, and the reaction was stirred at ambient temperature for 3 hours. The reaction was then heated to 50° C. overnight. The reaction was cooled, filtered, and purified by reverse phase HPLC to give a diastereomeric mixture of the desired product. Yield 11.6 mg. MS: 604.1 (M+H⁺); H¹ NMR (DMSO-d₆): δ (ppm) 0.45-0.75 (m, 4H), 2.75-2.92 (m, 1H), 3.12-3.30 (m, 1H), 3.53-3.68 (m, 1H), 4.85-5.25 (m, 3H), 6.51-6.45 (m, 1H), 6.92-7.34 (m, 6H), 7.79-8.01 (m, 5H), 8.27-8.62 (m, 4H).

### Example 9

### (2S,4R)-4-[4-(4-Cyclopropylcarbamoyl-phenyl)-thiazol-2-ylcarbamoyl]-2-pyridin-3-yl-thiazolidine-3-carboxylic acid benzyl ester (Compound 7009)

### (R)-2-Pyridin-3-yl-thiazolidine-4-carboxylic acid

To a solution of L-cysteine hydrochloride monohydrate (498.5 mg, 2.84 mmol) in distilled water (4.3 mL), potassium acetate (309.5 mg, 3.15 mmol) was added. Once the solids went into solution, methanol (4.3 mL) was added followed by pyridine-3-carbaldehyde (325 μL, 3.46 mmol). The reaction was stirred at ambient temperature for 5 hours. The solvent was removed, and no further purification steps were taken.

### (2S,4R)-2-Pyridin-3-yl-thiazolidine-3,4-dicarboxylic acid 3-benzyl ester & (2R,4R)-2-Pyridin-3-yl-thiazolidine-3,4-dicarboxylic acid 3-benzyl ester

(R)-2-Pyridin-3-yl-thiazolidine-4-carboxylic acid (524.0 mg, 2.49 mmol) was dissolved in DMF (15 mL). The solution was cooled to 0° C., and DIPEA (650 μL, 3.73 mmol) was added followed by benzyl chloroformate (530 μL, 3.71 mmol). The reaction was stirred at 0° C. and allowed to warm to ambient temperature overnight. The reaction was filtered and purified by reverse phase HPLC to give the two pure desired diastereomers.

### (2S,4R)-4-[4-(4-Cyclopropylcarbamoyl-phenyl)-thiazol-2-ylcarbamoyl]-2-pyridin-3-yl-thiazolidine-3-carboxylic acid benzyl ester (Compound 7009)

(2S,4R)-2-Pyridin-3-yl-thiazolidine-3,4-dicarboxylic acid 3-benzyl ester (58.4 mg, 0.17 mmol) was dissolved in DMF (1 mL). DIPEA (59 μL, 0.34 mmol) was added followed by HATU (64.5 mg, 0.17 mmol). The reaction was stirred at ambient temperature for 15 minutes. Then 4-(2-Amino-thiazol-4-yl)-N-cyclopropyl-benzamide (Example 1, 44.2 mg, 0.17 mmol) was added, and the reaction was stirred at ambient temperature overnight. The reaction was then heated to 50° C. overnight. The reaction was cooled, filtered, and purified by reverse phase HPLC to give the desired product. Yield 5.5 mg. MS: 586.1 (M+H⁺); H¹ NMR (DMSO-d₆): δ (ppm) 0.53-0.75 (m, 4H), 1.18-1.29 (m, 1H), 2.80-2.90 (m, 1H), 3.62-3.77 (m, 1H), 4.86-5.13 (m, 2H), 5.28-5.36 (m, 1H), 6.29-6.33 (s, 1H), 6.78-6.88 (m, 1H), 7.03-7.25 (m, 3H), 7.78-8.02 (m, 6H), 8.42-8.75 (m, 3H), 12.70-12.78 (s, 1H).

### Example 10

### (R)-4-[4-(4-Cyclopropylcarbamoyl-phenyl)-thiazol-2-ylcarbamoyl]-2-pyridin-2-yl-thiazolidine-3-carboxylic acid benzyl ester (Compound 7010)

### (R)-2-Pyridin-2-yl-thiazolidine-4-carboxylic acid

To a solution of L-cysteine hydrochloride monohydrate (492.8 mg, 2.81 mmol) in distilled water (4.3 mL), potassium acetate (338.0 mg, 3.44 mmol) was added. Once the solids went into solution, methanol (4.3 mL) was added followed by pyridine-2-carbaldehyde (322 μL, 3.37 mmol). The reaction was stirred at ambient temperature for 5 hours. The solvent was removed, and no further purification steps were taken.

### (R)-2-Pyridin-2-yl-thiazolidine-3,4-dicarboxylic acid 3-benzyl ester

(R)-2-Pyridin-2-yl-thiazolidine-4-carboxylic acid (500 mg, 2.38 mmol) was dissolved in DMF (15 mL). The solution was cooled to 0° C., and DIPEA (620 μL, 3.56 mmol) was added followed by benzyl chloroformate (510 μL, 3.57 mmol). The reaction was stirred at 0° C. and allowed to warm to ambient temperature overnight. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-pyridin-2-yl-thiazolidine-3-carboxylic acid benzyl ester (Compound 7010)

(R)-2-Pyridin-2-yl-thiazolidine-3,4-dicarboxylic acid 3-benzyl ester (58.9 mg, 0.17 mmol) was dissolved in DMF (1 mL). DIPEA (59 μL, 0.34 mmol) was added followed by HATU (64.5 mg, 0.17 mmol). The reaction was stirred at ambient temperature for 15 minutes. Then 4-(2-amino-thiazol-4-yl)-N-cyclopropyl-benzamide (Example 1, 45.8 mg, 0.18 mmol) was added, and the reaction was stirred at ambient temperature overnight. The reaction was then heated to 50° C. overnight. The reaction was cooled, filtered, and purified by reverse phase HPLC to give the desired product. Yield 5.0 mg. MS: 586.1 (M+H⁺); H¹ NMR (DMSO-d₆): δ (ppm) 0.55-0.74 (m, 4H), 2.80-2.92 (m, 1H), 3.60-3.77 (m, 1H), 4.86-5.18 (m, 3H), 6.40-6.44 (m, 1H), 6.84-6.92 (m, 1H), 7.05-7.25 (m, 4H), 7.50-7.59 (m, 1H), 7.66-8.09 (m, 8H), 8.44-8.50 (m, 1H), 8.85-8.12 (m, 1H).

### Example 11

### (S)-4-[4-(4-Cyclopropylcarbamoyl-phenyl)-thiazol-2-ylcarbamoyl]-2-pyridin-4-yl-oxazolidine-3-carboxylic acid benzyl ester (Compound 7011)

### 2-Pyridin-4-yl-oxazolidine-3,4-dicarboxylic acid 3-benzyl ester 4-methyl ester

A mixture of 2-benzyloxycarbonylamino-3-hydroxy-propionic acid methyl ester (Example 46, 0.275 g, 1.09 mmol), 4-pyridine dimethylacetal (0.51 g, 3.32 mmol), and TsOH (0.68 g, 3.57 mmol) in toluene (12 mL) was heated to reflux for 2 h. using a Dean Stark trap. The mixture was concentrated to about 1 mL then diluted with EtOAc (20 mL) and was washed with Sat. NaHCO₃, brine, dried (Na₂SO₄), and concentrated in vacuo to give the crude product. Purification by silica gel gave (0.170 g, 49.7%) of 2-pyridin-4-yl-oxazolidine-3,4-dicarboxylic acid 3-benzyl ester 4-methyl ester; 343.1 (M+H⁺).

### 2-Pyridin-4-yl-oxazolidine-3,4-dicarboxylic acid 3-benzyl ester

In a similar procedure as described in Example 46, hydrolysis of 2-pyridin-4-yl-oxazolidine-3,4-dicarboxylic acid 3-benzyl ester 4-methyl ester (0.17 g, 0.49 mmol) with NaOH (1M, 2 mL, 2 mmol) provided 2-pyridin-4-yl-oxazolidine-3,4-dicarboxylic acid 3-benzyl ester; MS: 329.1 (M+H⁺)

### (S)-4-[4-(4-Cyclopropylcarbamoyl-phenyl)-thiazol-2-ylcarbamoyl]-2-pyridin-4-yl-oxazolidine-3-carboxylic acid benzyl ester (Compound 7011)

In a similar procedure as described in Example 46, treatment of 2-pyridin-4-yl-oxazolidine-3,4-dicarboxylic acid 3-benzyl ester (0.134 g, 0.41 mmol) with HATU (0.16 g, 0.41 mmol), DIPEA (0.11 mL, 0.82 mmol), and 4-(2-amino-thiazol-4-yl)-N-cyclopropyl-benzamide (Example 1, 0.11 g, 0.41 mmol) provided the desired product. ¹HNMR (DMSO-d₆) δ (ppm) 12.74 (s, 1H), 8.80-8.68 (m, 2H), 8.46-8.45 (d, 1H), 8.03-7.60 (m, 7H), 7.30-7.09 (m, 4H), 6.93 (m, 1H), 6.35-6.15 (m, 1H), 5.13-4.94 (m, 3H), 4.42-4.26 (m, 2H), 2.88-2.82 (m, 1H), 1.23-1.11 (m, 3H), 0.73-0.55, (m, 4H); MS: 570.1 (M+H+).

### Example 12

### 4-[4-(4-Cyclopentylcarbamoyl-phenyl)-thiazol-2-ylcarbamoyl]-2-pyridin-4-yl-thiazolidine-3-carboxylic acid benzyl ester (Compound 7012)

From 30 mg of 4-(2-Amino-thiazol-4-yl)-N-cyclopentyl benzamide following procedure for Example 59. MS: 614.7 (M+H⁺); H¹ NMR (DMSO-d₆): δ (ppm) 12.94 (m, 1H), 8.68 (m, 2H), 8/56 (d, 1H), 7.65-8.18 (m, 7H), 6.82-7.32 (m, 6H), 6.49 (m, 1H), 6.31 (d, 2H), 5.32 (d, 1H), 5.14 (m, 2H), 3.45 (m, 1H), 2.81 (m, 1H), 0.51-0.76 (m, 8H).

### Example 13

### (R)-4-[4-(4-Cyclopropylcarbamoyl-phenyl)-thiazol-2-ylcarbamoyl]-2-(4-fluoro-phenyl)-thiazolidine-3-carboxylic acid benzyl ester (Compound 7013)

### (R)-2-(4-Fluoro-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 4-fluoro-benzaldehyde (325 μL, 3.06 mmol). The reaction was stirred at ambient temperature overnight. The solvents were removed, and no further purification steps were taken.

### (R)-2-(4-Fluoro-phenyl)-thiazolidine-3,4-dicarboxylic acid 3-benzyl ester

(R)-2-(4-Fluoro-phenyl)-thiazolidine-4-carboxylic acid (576.7 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(4-fluoro-phenyl)-thiazolidine-3-carboxylic acid benzyl ester (Compound 7013)

(R)-2-(4-Fluoro-phenyl)-thiazolidine-3,4-dicarboxylic acid 3-benzyl ester (89.9 mg, 0.25 mmol) was dissolved in DMF (1.5 mL). DIPEA (87 μL, 0.50 mmol) was added followed by HATU (94.8 mg, 0.25 mmol). The reaction was stirred at ambient temperature for 15 minutes. Then 4-(2-amino-thiazol-4-yl)-N-cyclopropyl-benzamide (Example 1, 64.0 mg, 0.25 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 17.4 mg. MS: 603.1 (M+H⁺); H¹ NMR (DMSO-d₆): δ (ppm) 0.39-0.66 (m, 4H), 2.68-2.81 (m, 1H), 2.99-3.13 (m, 1H), 2.86-2.98 (m, 1H), 4.75-5.01 (m, 3H), 6.18 (s, 1H), 6.83-7.18 (m, 7H), 7.66-7.91 (m, 7H), 8.31-8.39 (d, 1H).

### Example 14

### (R)-2-(4-Carboxy-phenyl)-4-[4-(4-cyclopropylcarbamoyl-phenyl)-thiazol-2-ylcarbamoyl]-thiazolidine-3-carboxylic acid benzyl ester (Compound 7014)

(R)-4-[4-(4-Cyclopropylcarbamoyl-phenyl)-thiazol-2-ylcarbamoyl]-2-(4-methoxycarbonyl-phenyl)-thiazolidine-3-carboxylic acid benzyl ester (Example 51, 28.7 mg, 0.04 mmol) was dissolved in THF (1 mL), methanol (500 μL), and distilled water (500 μL). Lithium hydroxide (12 mg, 0.50 mmol) was added, and the reaction was heated to 50° C. overnight. The reaction was cooled, filtered, and purified by reverse phase HPLC to give a diastereomeric mixture of the desired product. Yield 11.4 mg. MS: 629.1 (M+H⁺); H¹ NMR (DMSO-d₆): δ (ppm) 0.54-0.73 (m, 4H), 2.79-2.88 (m, 1H), 4.86-5.10 (m, 2H), 5.25-5.35 (m, 1H), 6.29-6.34 (d, 2H), 6.71-6.79 (m, 1H), 6.90-7.24 (m, 4H), 7.41-7.51 (m, 1H), 7.80-8.00 (m, 8H), 8.42-8.48 (m, 1H), 12.62-12.80 (m, 1H).

### Example 15

### (R)-4-[4-(4-Cyclopropylcarbamoyl-phenyl)-thiazol-2-ylcarbamoyl]-4,5-dihydro-[2,5′]bithiazolyl-3-carboxylic acid benzyl ester (Compound 7015)

### (R)-2,3,4,5-Tetrahydro-[2,5′]bithiazolyl-4-carboxylic acid

To a solution of L-cysteine hydrochloride (428 mg, 2.72 mmol) in distilled water (4.3 mL), potassium acetate (293 mg, 2.99 mmol) was added. Once the solids went into solution, methanol (4.3 mL) was added followed by thiazole-5-carbaldehyde (370 mg, 3.27 mmol). The reaction was stirred at ambient temperature for 4 hours. The solvent was removed, and no further purification steps were taken.

### (R)-4,5-Dihydro-[2,5′]bithiazolyl-3,4-dicarboxylic acid 3-benzyl ester

(R)-2,3,4,5-Tetrahydro-[2,5′]bithiazolyl-4-carboxylic acid (587 mg, 2.71 mmol) was dissolved in DMF (15 mL). The solution was cooled to 0° C., and DIPEA (710 μL, 4.08 mmol) was added followed by benzyl chloroformate (580 μL, 4.06 mmol). The reaction was stirred at 0° C. and allowed to warm to ambient temperature over 3 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-4,5-dihydro-[2,5′]bithiazolyl-3-carboxylic acid benzyl ester (Compound 7015)

(R)-4,5-Dihydro-[2,5′]bithiazolyl-3,4-dicarboxylic acid 3-benzyl ester (59 mg, 0.17 mmol) was dissolved in DMF (1 mL). DIPEA (59 μL, 0.34 mmol) was added followed by HATU (64 mg, 0.17 mmol). The reaction was stirred at ambient temperature for 15 minutes. Then 4-(2-amino-thiazol-4-yl)-N-cyclopropyl-benzamide (Example 1, 44 mg, 0.17 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. Yield 5.8 mg. MS: 592.1 (M+H⁺); H¹ NMR (DMSO-d₆): δ (ppm) 0.53-0.76 (m, 4H), 2.78-2.91 (m, 1H), 3.12-3.34 (m, 1H), 3.55-3.69 (m, 1H), 4.85-5.17 (m, 3H), 6.62-6.74 (m, 1H), 7.00-7.40 (m, 5H), 7.80-7.99 (m, 5H) 8.09-8.20 (m, 1H), 8.41-8.49 (m, 1H), 9.02 (s, 1H), 12.71 (s, 1H).

### Example 16

### (R)-4-[4-(4-Cyclopropylcarbamoyl-phenyl)-thiazol-2-ylcarbamoyl]-2-(3-methoxy-phenyl)-thiazolidine-3-carboxylic acid benzyl ester (Compound 7016)

### (R)-2-(3-Methoxy-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 3-methoxy-benzaldehyde (370 μL, 3.04 mmol). The reaction was stirred at ambient temperature overnight. The solvent was removed, and no further purification steps were taken.

### (R)-2-(3-Methoxy-phenyl)-thiazolidine-3,4-dicarboxylic acid 3-benzyl ester

(R)-2-(3-Methoxy-phenyl)-thiazolidine-4-carboxylic acid (607.3 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(3-methoxy-phenyl)-thiazolidine-3-carboxylic acid benzyl ester (Compound 7016)

(R)-2-(3-Methoxy-phenyl)-thiazolidine-3,4-dicarboxylic acid 3-benzyl ester (90.5 mg, 0.24 mmol) was dissolved in DMF (1.5 mL). DIPEA (85 μL, 0.49 mmol) was added followed by HATU (93.2 mg, 0.25 mmol). The reaction was stirred at ambient temperature for 15 minutes. Then 4-(2-amino-thiazol-4-yl)-N-cyclopropyl-benzamide (Example 1, 63.5 mg, 0.24 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 14.1 mg. MS: 615.1 (M+H⁺); H¹ NMR (DMSO-d₆): δ (ppm) 0.52-0.77 (m, 4H), 2.79-2.92 (m, 1H), 3.14-3.26 (m, 1H), 3.45-3.57 (m, 1H), 3.77 (s, 3H), 4.76-5.14 (m, 3H), 6.25 (s, 1H), 6.76-6.90 (m, 1H), 6.93-7.35 (m, 7H), 7.49 (s, 1H), 7.79-8.01 (m, 5H), 8.40-8.50 (m, 1H), 12.72 (s, 1H).

### Example 17

### (R)-2-(4-Cyano-phenyl)-4-[4-(4-cyclopropylcarbamoyl-phenyl)-thiazol-2-ylcarbamoyl]-thiazolidine-3-carboxylic acid benzyl ester (Compound 7017)

### (R)-2-(4-Cyano-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 4-formyl-benzonitrile (399 μL, 3.04 mmol). The reaction was stirred at ambient temperature overnight. The solvent was removed, and no further purification steps were taken.

### (R)-2-(4-Cyano-phenyl)-thiazolidine-3,4-dicarboxylic acid 3-benzyl ester

(R)-2-(4-Cyano-phenyl)-thiazolidine-4-carboxylic acid (594.5 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-2-(4-Cyano-phenyl)-4-[4-(4-cyclopropylcarbamoyl-phenyl)-thiazol-2-ylcarbamoyl]-thiazolidine-3-carboxylic acid benzyl ester (Compound 7017)

(R)-2-(4-Cyano-phenyl)-thiazolidine-3,4-dicarboxylic acid 3-benzyl ester (90.2 mg, 0.24 mmol) was dissolved in DMF (1 mL). DIPEA (86 μL, 0.49 mmol) was added followed by HATU (93.8 mg, 0.25 mmol). The reaction was stirred at ambient temperature for 15 minutes. Then 4-(2-amino-thiazol-4-yl)-N-cyclopropyl-benzamide (Example 1, 63.5 mg, 0.24 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 18.2 mg. MS: 610.2 (M+H⁺); H¹ NMR (DMSO-d₆): δ (ppm) 0.50-0.75 (m, 4H), 2.77-2.90 (m, 1H), 3.09-3.25 (m, 1H), 3.47-3.58 (m, 1H), 4.89-5.12 (m, 3H), 6.35 (s, 1H), 6.93-7.35 (m, 5H), 7.78-8.10 (m, 10H), 8.40-8.50 (m, 1H), 12.72-12.85 (m, 1H).

### Example 18

### 4-Phenylcarbamoyl-2-pyridin-3-yl-thiazolidine-3-carboxylic acid benzyl ester (Compound 7018)

From pyridine-3-carbaldehyde following procedure for Example 59. MS: 420.2 (M+H⁺); H¹ NMR (DMSO-d₆): δ (ppm) (HCl salt) 12.44 (dd, 1H), 8.33 (br s, 1H), 8.83 (m, 4H), 8.0-7.9 (m, 7H), 6.41 (d, 1 h), 5.16 (m, 1H), 4.98 (m, 2H), 3.64 (m, 1H), 3.21 (m, 1H), 1.23 (m, 2H).

### Example 19

### 2-[4-(4-Cyclopropylcarbamoyl-phenyl)-thiazol-2-ylcarbamoyl]-2,3-dihydro-indole-1-carboxylic acid benzyl ester (Compound 7019)

From 2,3-Dihydro-indole-1,2-dicarboxylic acid 1-benzyl ester following General Procedure A. Yield 10 mg. MS: 539.3 (M+H). H¹-NMR (DMSO-d₆): δ (ppm) 12.6 (s, 1H), 8.4 (m, 1H), 7.9-7.8 (m, 6H), 7.3-7.1 (m, 9H), 5.2-5.0 (m, 3H), 3.5 (m, 1H), 3.1 (m. 1H), 2.8 (m, 1H), 0.7-0.5 (m, 4H).

### Example 20

### (S)-2-[4-(4-Cyclopropylcarbamoyl-phenyl)-thiazol-2-ylcarbamoyl]-2,3-dihydro-indole-1-carboxylic acid tert-butyl ester (Compound 7020)

### (S)-2,3-Dihydro-indole-1,2-dicarboxylic acid 1-tert-butyl ester

(S)-2,3-Dihydro-1H-indole-2-carboxylic acid (207.4 mg, 1.27 mmol) was dissolved in DMF (10 mL). Triethylamine (375 μL, 2.69 mmol) was added followed by di-tert-butyl dicarbonate (300.7 mg, 1.38 mmol). The reaction was stirred at ambient temperature overnight. The reaction was filtered and purified by reverse phase HPLC to give the desired product.

### (S)-2-[4-(4-Cyclopropylcarbamoyl-phenyl)-thiazol-2-ylcarbamoyl]-2,3-dihydro-indole-1-carboxylic acid tert-butyl ester (Compound 7020)

(S)-2,3-Dihydro-indole-1,2-dicarboxylic acid 1-tert-butyl ester (108.0 mg, 0.41 mmol) was dissolved in DMF (4 mL). DIPEA (138 μL, 0.79 mmol) was added followed by HATU (150.0 mg, 0.39 mmol). The reaction was stirred at ambient temperature for 15 minutes. Then 4-(2-amino-thiazol-4-yl)-N-cyclopropyl-benzamide (Example 1, 103.5 mg, 0.40 mmol) was added, and the reaction was stirred at ambient temperature overnight. The reaction was then heated to 50° C. overnight. The reaction was cooled, filtered, and purified by reverse phase HPLC to give the desired product. Yield 9.0 mg. MS: 505.1 (M+H⁺); H¹ NMR (DMSO-d₆): δ (ppm)0.53-0.75 (m, 4H), 1.23-1.40 (s, 9H), 2.79-2.90 (m, 1H), 2.99-3.18 (m, 1H), 3.46-3.60 (m, 1H), 5.01-5.13 (m, 1H), 6.88-6.97 (m, 1H), 7.12-7.21 (m, 2H), 7.69-7.99 (m, 6H), 8.41-8.47 (d, 1H), 12.60-12.75 (s, 1H).

### Example 21

### (R)-4-[4-(4-Cyclopropylcarbamoyl-phenyl)-thiazol-2-ylcarbamoyl]-2-piperidin-3-yl-thiazolidine-3-carboxylic acid benzyl ester (Compound 7021)

### 3-((R)-4-Carboxy-thiazolidin-2-yl)-piperidine-1-carboxylic acid tert-butyl ester

To a solution of L-cysteine hydrochloride (400 mg, 2.54 mmol) in distilled water (4 mL), potassium acetate (275 mg, 2.80 mmol) was added. Once the solids went into solution, methanol (4 mL) was added followed by 3-formyl-piperidine-1-carboxylic acid tert-butyl ester (645 mg, 3.02 mmol). Precipitate crashed out of solution within 30 minutes. The solvent was removed, and no further purification steps were taken.

### (R)-2-(1-tert-Butoxycarbonyl-piperidin-3-yl)-thiazolidine-3,4-dicarboxylic acid 3-benzyl ester

To a solution of phenyl-methanol (360 μL, 3.48 mmol) in acetonitrile (6 mL), triethylamine (960 μL, 6.89 mmol) was added followed by carbonic acid bis-(2,5-dioxo-pyrrolidin-1-yl) ester (890 mg, 3.47 mmol). The reaction was stirred at ambient temperature for one hour. The solvent was removed, and the residue was redissolved in dry dichloromethane (6 mL). Then triethylamine (485 μL, 3.48 mmol), DMAP (5 mg), and 3-((R)-4-carboxy-thiazolidin-2-yl)-piperidine-1-carboxylic acid tert-butyl ester (735 mg, 2.32 mmol) were added. The reaction was stirred at ambient temperature for 5 hours. The reaction was filtered and purified by reverse phase HPLC to give a diastereomeric mixture of the desired product.

### 3-{(R)-3-Benzyloxycarbonyl-4-[4-(4-cyclopropylcarbamoyl-phenyl)-thiazol-2-ylcarbamoyl]-thiazolidin-2-yl}-piperidine-1-carboxylic acid tert-butyl ester

(R)-2-(1-tert-Butoxycarbonyl-piperidin-3-yl)-thiazolidine-3,4-dicarboxylic acid 3-benzyl ester (243 mg, 0.54 mmol) was dissolved in DMF (2.5 mL). DIPEA (187 μL, 1.07 mmol) was added followed by HATU (204 mg, 0.54 mmol). The reaction was stirred at ambient temperature for 15 minutes. Then 4-(2-amino-thiazol-4-yl)-N-cyclopropyl-benzamide (Example 1, 140 mg, 0.54 mmol) was added, and the reaction was stirred at ambient temperature for 1 hour. The reaction was then heated to 50° C. overnight. The solvent was removed, and no further purification steps were taken.

### (R)-4-[4-(4-Cyclopropylcarbamoyl-phenyl)-thiazol-2-ylcarbamoyl]-2-piperidin-3-yl-thiazolidine-3-carboxylic acid benzyl ester (Compound 7021)

A solution of 3-{(R)-3-benzyloxycarbonyl-4-[4-(4-cyclopropylcarbamoyl-phenyl)-thiazol-2-ylcarbamoyl]-thiazolidin-2-yl}-piperidine-1-carboxylic acid tert-butyl ester (186.8 mg, 0.27 mmol) in dichloromethane (750 μL) and trifluoroacetic acid (750 μL) was stirred at ambient temperature for 1.5 hours. The reaction was filtered and purified by reverse phase HPLC to give the desired product. Yield 10.0 mg. MS: 592.2 (M+H⁺); H¹ NMR (DMSO-d₆): δ (ppm) 0.50-0.88 (m, 4H), 1.15-2.05 (m, 5H), 2.19-2.42 (m, 1H), 2.65-2.93 (m, 3H), 2.98-3.31 (m, 2H), 3.38-3.60 (m, 2H), 4.80-5.26 (m, 4H), 7.01-7.46 (m, 5H), 7.80-8.02 (m, 5H), 8.23-8.52 (m, 2H), 8.70-8.86 (m, 1H).

### Example 22

### (R)-4-[4-(4-Cyclopropylcarbamoyl-phenyl)-thiazol-2-ylcarbamoyl]-2-pyrimidin-5-yl-thiazolidine-3-carboxylic acid benzyl ester (compound 7022)

### (R)-2-Pyrimidin-5-yl-thiazolidine-4-carboxylic acid

To a solution of L-cysteine hydrochloride (400 mg, 2.54 mmol) in distilled water (4 mL), potassium acetate (275 mg, 2.80 mmol) was added. Once the solids went into solution, methanol (4 mL) was added followed by pyrimidine-5-carbaldehyde (328.3 mg, 3.04 mmol). The reaction was allowed to stir at ambient temperature for one hour. The solvent was removed, and no further purification steps were taken.

### (R)-2-Pyrimidin-5-yl-thiazolidine-3,4-dicarboxylic acid 3-benzyl ester

To a solution of phenyl-methanol (360 μL, 3.48 mmol) in acetonitrile (6 mL), triethylamine (970 μL, 6.96 mmol) was added followed by carbonic acid bis-(2,5-dioxo-pyrrolidin-1-yl) ester (889.5 mg, 3.47 mmol). The reaction was stirred at ambient temperature for one hour. The solvent was removed, and the residue was redissolved in dry dichloromethane (6 mL). Then triethylamine (485 μL, 3.48 mmol), DMAP (5 mg), and (R)-2-pyrimidin-5-yl-thiazolidine-4-carboxylic acid (500 mg, 2.37 mmol) were added. The reaction was stirred at ambient temperature for 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-pyrimidin-5-yl-thiazolidine-3-carboxylic acid benzyl ester (compound 7022)

(R)-2-Pyrimidin-5-yl-thiazolidine-3,4-dicarboxylic acid 3-benzyl ester (249.3 mg, 0.72 mmol) was dissolved in DMF (5 mL). DIPEA (251 μL, 1.44 mmol) was added followed by HATU (274 mg, 0.72 mmol). The reaction was stirred at ambient temperature for 15 minutes. Then 4-(2-Amino-thiazol-4-yl)-N-cyclopropyl-benzamide (Example 1, 187 mg, 0.72 mmol) was added, and the reaction was stirred at ambient temperature for 1 hour. The reaction was then heated to 50° C. overnight. The reaction was cooled, filtered, and purified by reverse phase HPLC to give a diastereomeric mixture of the desired product. Yield 80.8 mg. MS: 587.1 (M+H⁺); H¹ NMR (DMSO-d₆): δ (ppm) 0.51-0.78 (m, 4H), 2.79-2.93 (m, 1H), 3.18-3.35 (m, 1H), 3.55-3.68 (m, 1H), 4.90-5.24 (m, 3H), 6.38 (s, 1H), 6.92-7.40 (m, 5H), 7.78-8.03 (m, 5H), 8.42-8.51 (d, 1H), 8.77 (s, 1H), 9.05-9.21 (m, 3H).

### Example 23

### (S)-2-(4-Phenyl-thiazol-2-ylcarbamoyl)-2,3-dihydro-indole-1-carboxylic acid benzyl ester (Compound 7023)

(S)-2,3-Dihydro-indole-1,2-dicarboxylic acid 1-benzyl ester (65.1 mg, 0.22 mmol) was dissolved in DMF (2 mL). DIPEA (77 μL, 0.44 mmol) was added followed by HATU (83.3 mg, 0.22 mmol). The reaction was stirred at ambient temperature for 15 minutes. Then 4-phenyl-thiazol-2-ylamine (39.2 mg, 0.22 mmol) was added, and the reaction was stirred at ambient temperature overnight. The reaction was purified by silica gel chromatography to give the desired product. Yield 5.7 mg. MS: 456.1 (M+H⁺); H¹ NMR (DMSO-d₆): δ (ppm) 0.78-0.97 (m, 2H), 1.06-1.26 (m, 2H), 3.07-3.22 (m, 1H), 3.50-3.66 (m, 1H) 5.01-5.14 (m, 3H), 6.90-7.48 (m, 11H), 7.62-7.70 (s, 1H), 7.73-7.92 (m, 3H), 12.70-12.75 (s, 1H).

### Example 24

### (R)-4-[4-(4-Cyclopropylcarbamoyl-phenyl)-thiazol-2-ylcarbamoyl]-2-methyl-thiazolidine-3-carboxylic acid benzyl ester (Compound 7024)

### (R)-2-Methyl-thiazolidine-3,4-dicarboxylic acid 3-benzyl ester

(R)-2-Methyl-thiazolidine-4-carboxylic acid (405.2 mg, 2.75 mmol) was dissolved in DMF (10 mL) and distilled water (10 mL). The solution was cooled to 0° C., and DIPEA (985 μL, 5.65 mmol) was added followed by benzyl chloroformate (585 μL, 4.10 mmol). The reaction was stirred at 0° C. and allowed to warm to ambient temperature overnight. The reaction was filtered and purified by reverse phase HPLC to give the desired product.

### (R)-4-[4-(4-Cyclopropylcarbamoyl-phenyl)-thiazol-2-ylcarbamoyl]-2-methyl-thiazolidine-3-carboxylic acid benzyl ester (Compound 7024)

(R)-2-Methyl-thiazolidine-3,4-dicarboxylic acid 3-benzyl ester (54.3 mg, 0.19 mmol) was dissolved in DMF (2 mL). DIPEA (67 μL, 0.38 mmol) was added followed by HATU (73.2 mg, 0.19 mmol). The reaction was stirred at ambient temperature for 15 minutes. Then 4-(2-amino-thiazol-4-yl)-N-cyclopropyl-benzamide (Example 1, 49.8 mg, 0.19 mmol) was added, and the reaction was stirred at ambient temperature overnight. The reaction was then heated to 50° C. for 7 hours. The reaction was cooled, filtered, and purified by reverse phase HPLC to give the desired product. Yield 23.3 mg. MS: 523.1 (M+H⁺); H¹ NMR (DMSO-d₆): δ (ppm) 0.54-0.74 (m, 4H), 1.53-1.61 (m, 3H), 2.69-2.91 (m, 2H), 4.80-5.14 (m, 3H), 5.21-5.33 (m, 1H), 7.02-7.41 (m, 5H), 7.78-7.98 (m, 5H), 8.40-8.47 (m, 1H), 12.60-12.65 (s, 1H).

### Example 25

### (R)-4-[4-(4-Cyclopropylcarbamoyl-phenyl)-thiazol-2-ylcarbamoyl]-2-(tetrahydro-pyran-4-yl)-thiazolidine-3-carboxylic acid benzyl ester (compound 7025)

### (R)-2-(Tetrahydro-pyran-4-yl)-thiazolidine-4-carboxylic acid

To a solution of L-cysteine hydrochloride (400.2 mg, 2.54 mmol) in distilled water (4 mL), potassium acetate (293.4 mg, 2.99 mmol) was added. Once the solids went into solution, methanol (4 mL) was added followed by tetrahydro-pyran-4-carbaldehyde (342.0 mg, 3.00 mmol). Precipitate crashed out of solution within 30 minutes. The solvent was removed, and no further purification steps were taken.

### (R)-2-(Tetrahydro-pyran-4-yl)-thiazolidine-3,4-dicarboxylic acid 3-benzyl ester

To a solution of phenyl-methanol (133 μL, 1.29 mmol) in acetonitrile (3 mL), triethylamine (357 μL, 2.56 mmol) was added followed by carbonic acid bis-(2,5-dioxo-pyrrolidin-1-yl) ester (328 mg, 1.28 mmol). The reaction was stirred at ambient temperature for one hour. The solvent was removed, and the residue was redissolved in dry dichloromethane (3 mL). Then triethylamine (178 μL, 1.28 mmol), DMAP (5 mg), and (R)-2-(tetrahydro-pyran-4-yl)-thiazolidine-4-carboxylic acid (185 mg, 0.85 mmol) were added. The reaction was stirred at ambient temperature for 4 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(tetrahydro-pyran-4-yl)-thiazolidine-3-carboxylic acid benzyl ester (Compound 7025)

(R)-2-(Tetrahydro-pyran-4-yl)-thiazolidine-3,4-dicarboxylic acid 3-benzyl ester (60 mg, 0.17 mmol) was dissolved in DMF (1 mL). DIPEA (57 μL, 0.33 mmol) was added followed by HATU (62.4 mg, 0.16 mmol). The reaction was stirred at ambient temperature for 15 minutes. Then 4-(2-amino-thiazol-4-yl)-N-cyclopropyl-benzamide (Example 1, 42.6 mg, 0.16 mmol) was added, and the reaction was stirred at ambient temperature for 3 hours. The reaction was then heated to 50° C. overnight. The reaction was cooled, filtered, and purified by reverse phase HPLC to give a diastereomeric mixture of the desired product. Yield 7.3 mg. MS: 593.1 (M+H⁺); H¹ NMR (DMSO-d₆): δ (ppm) 0.38-0.80 (m, 4H), 1.11-1.92 (m, 8H), 2.78-2.93 (m, 1H), 3.05-3.30 (m, 2H), 3.76-4.00 (m, 2H), 4.76-5.20 (m, 4H), 6.99-7.46 (m, 5H), 7.77-8.02 (m, 5H), 8.41-8.50 (d, 1H).

### Example 26

### 4-(4-Phenyl-thiazol-2-ylcarbamoyl)-2-pyridin-4-yl-thiazolidine-3-carboxylic acid benzyl ester (Compound 7026)

2-Pyridin-4-yl-thiazolidine-3,4-dicarboxylic acid 3-benzyl ester (100 mg, 0.29 mmol) was combined with HATU (100 mg, 0.29 mmol) and DIPEA (0.1 mL, 0.6 mmol) in 6 mL of DMF. This solution was stirred at room temperature for 30 minutes. To this solution was added thiourea (220 mg, 2.9 mmol). Reaction mixture was stirred at room temperature overnight to yield the crude product, 2-pyridin-4-yl-4-thioureidocarbonyl-thiazolidine-3-carboxylic acid benzyl ester, which was purified using reverse phase HPLC. A portion of this purified material (30 mg, 0.074 mmol) was combined with 2-Bromo-1-phenyl-ethanone (14.8 mg, 0.074 mmol) in 6 mL of dry EtOH. To this solution was then added NaOAc (9.1 mg, 0.11 mmol). The reaction mixture was stirred at room temperature for 1 hour and then evaporated to dryness. The residue was redissolved in 10 mL of DMF, filtered, and purified using reverse phase HPLC.

MS: 503.7 (M+H⁺); H¹ NMR (DMSO-d₆): δ (ppm) (HCl salt) 12.79 (d, 2H), 8.90-8.50 (m, 4H), 7.90-0.670 (m, 10H), 6.44 (d, 1H), 5.42 (m, 2H), 4.60 (m, 1H), 3.58 (m, 1H), 2.83 (m, 2H), 2.33 (m, 1H).

### Example 27

### (2S,4R)-2-[4-(4-Cyclopropylcarbamoyl-phenyl)-thiazol-2-ylcarbamoyl]-4-phenyl-pyrrolidine-1-carboxylic acid tert-butyl ester (Compound 7027)

(2S,4R)-4-Phenyl-pyrrolidine-1,2-dicarboxylic acid 1-tert-butyl ester (54.4 mg, 0.19 mmol) was dissolved in DMF (1.5 mL). DIPEA (66 μL, 0.38 mmol) was added followed by HATU (71.2 mg, 0.19 mmol). The reaction was stirred at ambient temperature for 15 minutes. Then 4-(2-amino-thiazol-4-yl)-N-cyclopropyl-benzamide (Example 1, 48.7 mg, 0.19 mmol) was added, and the reaction was stirred at ambient temperature for 8 hours. The reaction was then heated to 50° C. overnight. The reaction was cooled, filtered, and purified by reverse phase HPLC to give the desired product. Yield 31.2 mg. MS: 533.2 (M+H⁺); H¹ NMR (DMSO-d₆): δ (ppm) 0.54-0.75 (m, 4H), 1.28-1.30 (s, 6H), 1.33-1.45 (s, 3H), 1.90-2.09 (m, 1H), 2.55-2.65 (m, 1H), 2.70-2.80 (m, 1H), 3.30-3.48 (m, 1H), 3.80-3.98 (m, 1H), 4.45-4.57 (m, 1H), 7.18-7.36 (m, 5H), 7.75-7.99 (m, 5H), 8.41-8.47 (m, 1H), 12.50-12.60 (m, 1H).

### Example 28

### (2R,4R)-4-[4-(4-Cyclopropylcarbamoyl-phenyl)-thiazol-2-ylcarbamoyl]-2-pyridin-3-yl-thiazolidine-3-carboxylic acid benzyl ester (Compound 7028)

(2R,4R)-2-Pyridin-3-yl-thiazolidine-3,4-dicarboxylic acid 3-benzyl ester (253.4 mg, 0.74 mmol) was dissolved in DMF (4 mL). DIPEA (256 μL, 1.47 mmol) was added followed by HATU (278.3 mg, 0.73 mmol). The reaction was stirred at ambient temperature for 15 minutes. Then 4-(2-amino-thiazol-4-yl)-N-cyclopropyl-benzamide (Example 1, 190.9 mg, 0.74 mmol) was added, and the reaction was stirred at ambient temperature for 4 hours. The reaction was then heated to 50° C. overnight. The reaction was cooled, filtered, and purified by reverse phase HPLC to give the desired product. The product was then converted to the HCl salt. After dissolving the product in a minimum amount of acetonitrile and cooling the solution in dry ice, 2.0M HCl in diethyl ether was added until precipitate crashed out of solution. The mixture was centrifuged, and the liquid was decanted. Additional cold diethyl ether was added, and the mixture was again centrifuged and the liquid decanted. The resulting solid was dried to give the HCl salt of the desired product. Yield 6.0 mg. MS: 586.1 (M+H⁺); H¹ NMR (DMSO-d₆): δ (ppm) 0.53-0.74 (m, 4H), 1.19-1.29 (m, 1H), 2.79-2.90 (m, 1H), 3.15-3.35 (m, 1H), 4.91-5.13 (m, 3H), 6.36-6.41 (s, 1H), 6.95-7.29 (m, 4H), 7.48-7.63 (m, 1H), 7.80-7.98 (m, 5H), 8.28-8.64 (m, 2H), 8.93-9.05 (m, 1H), 12.75-12.82 (s, 1H).

### Example 29

### (R)-2-(2-Chloro-pyridin-4-yl)-4-[4-(4-cyclopropylcarbamoyl-phenyl)-thiazol-2-ylcarbamoyl]-thiazolidine-3-carboxylic acid benzyl ester (Compound 7029)

### (R)-2-(2-Chloro-pyridin-4-yl)-thiazolidine-4-carboxylic acid

To a solution of L-cysteine hydrochloride (403.7 mg, 2.56 mmol) in distilled water (4 mL), potassium acetate (306.2 mg, 3.12 mmol) was added. Once the solids went into solution, methanol (4 mL) was added followed by 2-chloro-pyridine-4-carbaldehyde (432.2 mg, 3.06 mmol). Precipitate crashed out of solution within one hour, and the reaction was stirred at ambient temperature overnight. The solvent was removed, and no further purification steps were taken.

### (R)-2-(2-Chloro-pyridin-4-yl)-thiazolidine-3,4-dicarboxylic acid 3-benzyl ester

(R)-2-(2-Chloro-pyridin-4-yl)-thiazolidine-4-carboxylic acid (627 mg, 2.56 mmol) was dissolved in DMF (12 mL). The solution was cooled to 0° C., and DIPEA (665 μL, 3.82 mmol) was added followed by benzyl chloroformate (905 μL, 6.34 mmol). The reaction was stirred at 0° C. and allowed to warm to ambient temperature over 5 hours. The reaction was filtered and purified by reverse phase HPLC to give a diastereomeric mixture of the desired product.

### (R)-2-(2-Chloro-pyridin-4-yl)-4-[4-(4-cyclopropylcarbamoyl-phenyl)-thiazol-2-ylcarbamoyl]-thiazolidine-3-carboxylic acid benzyl ester (Compound 7029)

(R)-2-(2-Chloro-pyridin-4-yl)-thiazolidine-3,4-dicarboxylic acid 3-benzyl ester (91.8 mg, 0.24 mmol) was dissolved in DMF (1.5 mL). DIPEA (84 μL, 0.48 mmol) was added followed by HATU (92.4 mg, 0.24 mmol). The reaction was stirred at ambient temperature for 15 minutes. Then 4-(2-amino-thiazol-4-yl)-N-cyclopropyl-benzamide (Example 1, 63.0 mg, 0.24 mmol) was added, and the reaction was stirred at ambient temperature for 4 hours. The reaction was then heated to 50° C. overnight. The reaction was cooled, filtered, and purified by reverse phase HPLC to give a diastereomeric mixture of the desired product. The product was then converted to the HCl salt. After dissolving the product in a minimum amount of acetonitrile and cooling the solution in dry ice, 2.0M HCl in diethyl ether was added until precipitate crashed out of solution. The mixture was centrifuged, and the liquid was decanted. Additional cold diethyl ether was added, and the mixture was again centrifuged and the liquid decanted. The resulting solid was dried to give the HCl salt of the desired product. Yield 5.2 mg. MS: 622.1 (M+H⁺); H¹ NMR (DMSO-d₆): δ (ppm) 0.49-0.76 (m, 4H), 2.76-2.92 (m, 1H), 3.17-3.20 (m, 1H), 3.45-3.60 (m, 1H), 4.86-5.20 (m, 3H), 6.30 (s, 1H), 6.98-7.34 (m, 5H), 7.75-8.04 (m, 7H), 8.35-8.51 (m, 2H).

### Example 30

### (R)-4-[4-(4-Cyclopropylcarbamoyl-phenyl)-thiazol-2-ylcarbamoyl]-2-(4-methoxy-phenyl)-thiazolidine-3-carboxylic acid benzyl ester (Compound 7030)

### (R)-2-(4-Methoxy-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 4-methoxy-benzaldehyde (370 μL, 3.04 mmol). The reaction was stirred at ambient temperature overnight. The solvent was removed, and no further purification steps were taken.

### (R)-2-(4-Methoxy-phenyl)-thiazolidine-3,4-dicarboxylic acid 3-benzyl ester

(R)-2-(4-Methoxy-phenyl)-thiazolidine-4-carboxylic acid (607.3 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(4-methoxy-phenyl)-thiazolidine-3-carboxylic acid benzyl ester (Compound 7030)

(R)-2-(4-Methoxy-phenyl)-thiazolidine-3,4-dicarboxylic acid 3-benzyl ester (91.3 mg, 0.24 mmol) was dissolved in DMF (1.5 mL). DIPEA (85 μL, 0.49 mmol) was added followed by HATU (94.0 mg, 0.25 mmol). The reaction was stirred at ambient temperature for 15 minutes. Then 4-(2-amino-thiazol-4-yl)-N-cyclopropyl-benzamide (Example 1, 63.2 mg, 0.24 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 6.1 mg. MS: 615.2 (M+H⁺); H¹ NMR (DMSO-d₆): δ (ppm) 0.52-0.77 (m, 4H), 2.80-2.92 (m, 1H), 3.10-3.23 (m, 1H), 3.45-3.55 (m, 1H), 3.76 (s, 3H), 4.86-5.11 (m, 3H), 6.23 (s, 1H), 6.80-7.38 (m, 8H), 7.63-7.75 (d, 2H), 7.80-8.03 (m, 5H), 8.42-8.50 (d, 1H), 12.71 (s, 1H).

### Example 31

### (R)-4-[4-(4-Cyclopropylcarbamoyl-phenyl)-thiazol-2-ylcarbamoyl]-2-pyridin-4-yl-thiazolidine-3-carboxylic acid benzyl ester (compound 7031)

### (R)-2-Pyridin-4-yl-thiazolidine-4-carboxylic acid

To a solution of L-cysteine hydrochloride monohydrate (499.0 mg, 2.84 mmol) in distilled water (4.3 mL), potassium acetate (317.3 mg, 3.23 mmol) was added. Once the solids went into solution, methanol (4.3 mL) was added followed by pyridine-4-carbaldehyde (325 μL, 3.45 mmol). The reaction was stirred at ambient temperature for 5 hours. The solvent was removed, and no further purification steps were taken.

### (R)-2-Pyridin-4-yl-thiazolidine-3,4-dicarboxylic acid 3-benzyl ester

(R)-2-Pyridin-4-yl-thiazolidine-4-carboxylic acid (500 mg, 2.38 mmol) was dissolved in DMF (15 mL). The solution was cooled to 0° C., and DIPEA (620 μL, 3.56 mmol) was added followed by benzyl chloroformate (510 μL, 3.57 mmol). The reaction was stirred at 0° C. and allowed to warm to ambient temperature overnight. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-pyridin-4-yl-thiazolidine-3-carboxylic acid benzyl ester (compound 7031)

(R)-2-Pyridin-4-yl-thiazolidine-3,4-dicarboxylic acid 3-benzyl ester (58.6 mg, 0.17 mmol) was dissolved in DMF (1 mL). DIPEA (59 μL, 0.34 mmol) was added followed by HATU (64.5 mg, 0.17 mmol). The reaction was stirred at ambient temperature for 15 minutes. Then 4-(2-amino-thiazol-4-yl)-N-cyclopropyl-benzamide (Example 1, 45.3 mg, 0.17 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. The product was then converted to the HCl salt. After dissolving the product in a minimum amount of acetonitrile and cooling the solution in dry ice, 2.0M HCl in diethyl ether was added until precipitate crashed out of solution. The mixture was centrifuged, and the liquid was decanted. Additional cold diethyl ether was added, and the mixture was again centrifuged and the liquid decanted. The resulting solid was dried to give the HCl salt of the desired product. Yield 6.6 mg. MS: 586.1 (M+H⁺); H¹ NMR (DMSO-d₆): δ (ppm) 0.51-0.76 (m, 4H), 2.79-2.92 (m, 1H), 3.35-3.50 (m, 1H), 4.85-5.14 (m, 2H), 5.30-5.38 (d, 1H), 6.31-6.36 (d, 0.6H), 6.39-6.43 (s, 0.4H), 6.82-7.31 (m, 6H), 7.65-8.17 (m, 7H), 8.42-8.50 (d, 1H), 8.59-8.82 (m, 2H), 12.70-12.85 (m, 1H).

### Example 32

### 4-[4-(4-Cyclopropylcarbamoyl-phenyl)-thiazol-2-ylcarbamoyl]-2-pyridin-3-yl-thiazolidine-3-carboxylic acid tert-butyl ester (Compound 7032)

### (2R,4R)-2-Pyridin-3-yl-thiazolidine-3,4-dicarboxylic acid 3-tert-butyl ester and (2S,4R)-2-Pyridin-3-yl-thiazolidine-3,4-dicarboxylic acid 3-tert-butyl ester

A solution of (R)-2-pyridin-3-yl-thiazolidine-4-carboxylic acid (650 mg, 3.09 mmol) and di-tert-butyl dicarbonate (806 mg, 3.69 mmol) in dichloromethane (15 mL) was stirred at ambient temperature for 3 hours. The reaction was filtered and purified by reverse phase HPLC to give (2R,4R)-2-pyridin-3-yl-thiazolidine-3,4-dicarboxylic acid 3-tert-butyl ester and (2S,4R)-2-pyridin-3-yl-thiazolidine-3,4-dicarboxylic acid 3-tert-butyl ester separately.

### 4-[4-(4-Cyclopropylcarbamoyl-phenyl)-thiazol-2-ylcarbamoyl]-2-pyridin-3-yl-thiazolidine-3-carboxylic acid tert-butyl ester (Compound 7032)

One of the single diastereomer, either (2R,4R)-2-pyridin-3-yl-thiazolidine-3,4-dicarboxylic acid 3-tert-butyl ester or (2S,4R)-2-pyridin-3-yl-thiazolidine-3,4-dicarboxylic acid 3-tert-butyl ester (57.5 mg, 0.19 mmol), was dissolved in DMF (1 mL). DIPEA (63 μL, 0.36 mmol) was added followed by HATU (69.3 mg, 0.18 mmol). The reaction was stirred at ambient temperature for 15 minutes. Then 4-(2-amino-thiazol-4-yl)-N-cyclopropyl-benzamide (Example 1, 47.1 mg, 0.18 mmol) was added, and the reaction was stirred at ambient temperature for 5 hours. The reaction was then heated to 50° C. overnight. The reaction was cooled, filtered, and purified by reverse phase HPLC to give the desired product. Yield 5.0 mg. MS: 552.1 (M+H⁺); H¹ NMR (DMSO-d₆): δ (ppm) 0.52-0.78 (m, 4H), 1.04-1.30 (d, 9H), 2.79-2.90 (m, 1H), 5.11-5.30 (m, 1H), 7.49-7.60 (m, 1H), 7.77-8.07 (m, 6H), 8.39-8.71 (m, 3H), 12.65-12.74 (m, 1H).

### Example 33

### (R)-2-(3-Cyano-phenyl)-4-[4-(4-cyclopropylcarbamoyl-phenyl)-thiazol-2-ylcarbamoyl]-thiazolidine-3-carboxylic acid benzyl ester (Compound 7033)

### (R)-2-(3-Cyano-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 3-formyl-benzonitrile (399 μL, 3.04 mmol). The reaction was stirred at ambient temperature overnight. The solvent was removed, and no further purification steps were taken

### (R)-2-(3-Cyano-phenyl)-thiazolidine-3,4-dicarboxylic acid 3-benzyl ester

(R)-2-(3-Cyano-phenyl)-thiazolidine-4-carboxylic acid (594.5 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-2-(3-Cyano-phenyl)-4-[4-(4-cyclopropylcarbamoyl-phenyl)-thiazol-2-ylcarbamoyl]-thiazolidine-3-carboxylic acid benzyl ester (Compound 7033)

(R)-2-(3-Cyano-phenyl)-thiazolidine-3,4-dicarboxylic acid 3-benzyl ester (60 mg, 0.16 mmol) was dissolved in DMF (1 mL). DIPEA (57 μL, 0.33 mmol) was added followed by HATU (62 mg, 0.16 mmol). The reaction was stirred at ambient temperature for 15 minutes. Then 4-(2-amino-thiazol-4-yl)-N-cyclopropyl-benzamide (Example 1, 43 mg, 0.17 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 10.5 mg. MS: 610.1 (M+H⁺); H¹ NMR (DMSO-d₆): δ (ppm) 0.46-0.72 (m, 4H), 2.71-2.87 (m, 1H), 3.07-3.19 (m, 1H), 4.83-5.08 (m, 3H), 6.26 (s, 1H), 6.86-7.24 (m, 5H), 7.47-7.59 (m, 1H), 7.65-8.08 (m, 7H), 8.14-8.23 (m, 1H), 8.35-8.42 (d, 1H).

### Example 34

### (R)-2-(3-Carboxy-phenyl)-4-[4-(4-cyclopropylcarbamoyl-phenyl)-thiazol-2-ylcarbamoyl]-thiazolidine-3-carboxylic acid benzyl ester (Compound 7034)

(R)-4-[4-(4-Cyclopropylcarbamoyl-phenyl)-thiazol-2-ylcarbamoyl]-2-(3-methoxycarbonyl-phenyl)-thiazolidine-3-carboxylic acid benzyl ester (Example 57, 25.2 mg, 0.04 mmol) was dissolved in THF (600 μL), methanol (300 μL), and distilled water (300 μL). Lithium hydroxide (12.9 mg, 0.54 mmol) was added, and the reaction was heated to 50° C. overnight. The reaction was cooled, filtered, and purified by reverse phase HPLC to give a diastereomeric mixture of the desired product. Yield 9.4 mg. MS: 629.1 (M+H⁺); H¹ NMR (DMSO-d₆): δ (ppm) 0.49-0.78 (m, 4H), 2.75-2.82 (m, 1H), 4.81-5.15 (m, 3H), 6.30-6.40 (m, 1H), 6.90-7.33 (m, 4H), 7.39-7.70 (m, 1H), 7.75-8.28 (m, 9H), 8.40-8.40-8.51 (m, 1H), 12.65-12.12.75 (m, 1H).

### Example 35

### (R)-2-Cyclohexyl-4-[4-(4-cyclopropylcarbamoyl-phenyl)-thiazol-2-ylcarbamoyl]-thiazolidine-3-carboxylic acid benzyl ester (Compound 7035)

### (R)-2-Cyclohexyl-thiazolidine-4-carboxylic acid

To a solution of L-cysteine hydrochloride (430.4 mg, 2.73 mmol) in distilled water (4.3 mL), potassium acetate (300 mg, 3.06 mmol) was added. Once the solids went into solution, methanol (4.3 mL) was added followed by cyclohexanecarbaldehyde (395 mg, 3.26 mmol). The reaction was stirred at ambient temperature for 4 hours. The solvent was removed, and no further purification steps were taken.

### (R)-2-Cyclohexyl-thiazolidine-3,4-dicarboxylic acid 3-benzyl ester

(R)-2-Cyclohexyl-thiazolidine-4-carboxylic acid (588 mg, 2.73 mmol) was dissolved in DMF (15 mL). The solution was cooled to 0° C., and DIPEA (715 μL, 4.10 mmol) was added followed by benzyl chloroformate (585 μL, 4.10 mmol). The reaction was stirred at 0° C. and allowed to warm to ambient temperature over 3 hours. The reaction was filtered and purified by reverse phase HPLC to give a diastereomeric mixture of the desired product.

### (R)-2-Cyclohexyl-4-[4-(4-cyclopropylcarbamoyl-phenyl)-thiazol-2-ylcarbamoyl]-thiazolidine-3-carboxylic acid benzyl ester (Compound 7035)

(R)-2-Cyclohexyl-thiazolidine-3,4-dicarboxylic acid 3-benzyl ester (59 mg, 0.17 mmol) was dissolved in DMF (1 mL). DIPEA (59 μL, 0.34 mmol) was added followed by HATU (64 mg, 0.17 mmol). The reaction was stirred at ambient temperature for 15 minutes. Then 4-(2-amino-thiazol-4-yl)-N-cyclopropyl-benzamide (Example 1, 44 mg, 0.17 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. Yield 5.1 mg. MS: 591.2 (M+H⁺); H¹ NMR (DMSO-d₆): δ (ppm) 0.53-0.75 (m, 4H), 0.80-1.30 (m, 6H), 1.55-2.05 (m, 4H), 2.80-2.91 (m, 1H), 3.05-3.23 (m, 1H), 4.73-5.15 (m, 4H), 6.53 (s, 1H), 7.00-7.45 (m, 5H), 7.74-8.00 (m, 5H), 8.40-8.48 (m, 1H), 12.60 (s, 1H).

### Example 36

### (R)-4-[4-(4-Cyclopropylcarbamoyl-phenyl)-thiazol-2-ylcarbamoyl]-2-oxazol-5-yl-thiazolidine-3-carboxylic acid benzyl ester (Compound 7036)

### (R)-2-Oxazol-5-yl-thiazolidine-4-carboxylic acid

To a solution of L-cysteine hydrochloride (446.9 mg, 2.84 mmol) in distilled water (4.3 mL), potassium acetate (319.8 mg, 3.26 mmol) was added. Once the solids went into solution, methanol (4.3 mL) was added followed by oxazole-5-carbaldehyde (339.0 mg, 3.49 mmol). The reaction was stirred at ambient temperature for 4 hours. The solvent was removed, and no further purification steps were taken.

### (R)-2-Oxazol-5-yl-thiazolidine-3,4-dicarboxylic acid 3-benzyl ester

(R)-2-Oxazol-5-yl-thiazolidine-4-carboxylic acid (568 mg, 2.84 mmol) was dissolved in DMF (15 mL). The solution was cooled to 0° C., and DIPEA (740 μL, 4.25 mmol) was added followed by benzyl chloroformate (610 μL, 4.27 mmol). The reaction was stirred at 0° C. and allowed to warm to ambient temperature over 3 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-oxazol-5-yl-thiazolidine-3-carboxylic acid benzyl ester (Compound 7036)

(R)-2-Oxazol-5-yl-thiazolidine-3,4-dicarboxylic acid 3-benzyl ester (58 mg, 0.17 mmol) was dissolved in DMF (1 mL). DIPEA (61 μL, 0.35 mmol) was added followed by HATU (66 mg, 0.17 mmol). The reaction was stirred at ambient temperature for 15 minutes. Then 4-(2-amino-thiazol-4-yl)-N-cyclopropyl-benzamide (Example 1, 45 mg, 0.17 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. Yield 5.2 mg. MS: 576.2 (M+H⁺); H¹ NMR (DMSO-d₆): δ (ppm) 0.52-0.76 (m, 4H), 1.18-1.28 (m, 1H), 2.75-2.90 (m, 1H), 4.79-5.18 (m, 3H), 6.42-6.47 (m, 0.55H), 6.47-6.53 (m, 0.43H), 6.98-7.47 (m, 8H), 7.78-8.02 (m, 4H), 8.32-8.38 (m, 1H), 8.40-8.48 (m, 1H).

### Example 37

### (R)-4-[4-(4-Cyclopropylcarbamoyl-phenyl)-thiazol-2-ylcarbamoyl]-2-(2-fluoro-phenyl)-thiazolidine-3-carboxylic acid benzyl ester (compound 7037)

### (R)-2-(2-Fluoro-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 2-fluoro-benzaldehyde (325 μL, 3.06 mmol). The reaction was stirred at ambient temperature overnight. The solvent was removed, and no further purification steps were taken.

### (R)-2-(2-Fluoro-phenyl)-thiazolidine-3,4-dicarboxylic acid 3-benzyl ester

(R)-2-(2-Fluoro-phenyl)-thiazolidine-4-carboxylic acid (576.7 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(2-fluoro-phenyl)-thiazolidine-3-carboxylic acid benzyl ester (compound 37)

(R)-2-(2-Fluoro-phenyl)-thiazolidine-3,4-dicarboxylic acid 3-benzyl ester (60 mg, 0.17 mmol) was dissolved in DMF (1 mL). DIPEA (58 μL, 0.33 mmol) was added followed by HATU (63 mg, 0.17 mmol). The reaction was stirred at ambient temperature for 15 minutes. Then 4-(2-amino-thiazol-4-yl)-N-cyclopropyl-benzamide (Example 1, 43 mg, 0.17 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 13.0 mg. MS: 603.1 (M+H⁺); H¹ NMR (DMSO-d₆): δ (ppm) 0.52-0.78 (m, 4H), 2.78-2.92 (m, 1H), 3.11-3.29 (m, 1H), 4.85-5.14 (m, 3H), 6.34-6.38 (m, 1H), 6.90-7.42 (m, 9H), 7.78-8.01 (m, 5H), 8.25-8.37 (m, 1H), 8.41-8.49 (m, 1H), 12.77 (s, 1H).

### Example 38

### (2S,4S)-2-[4-(4-Cyclopropylcarbamoyl-phenyl)-thiazol-2-ylcarbamoyl]-4-phenyl-pyrrolidine-1-carboxylic acid tert-butyl ester (Compound 7038)

(2S,4S)-4-Phenyl-pyrrolidine-1,2-dicarboxylic acid 1-tert-butyl ester (54.1 mg, 0.19 mmol) was dissolved in DMF (2 mL). DIPEA (66 μL, 0.38 mmol) was added followed by HATU (71.6 mg, 0.19 mmol). The reaction was stirred at ambient temperature for 15 minutes. Then 4-(2-amino-thiazol-4-yl)-N-cyclopropyl-benzamide (Example 1, 48.7 mg, 0.19 mmol) was added, and the reaction was stirred at ambient temperature for 6 hours. The reaction was then heated to 45° C. overnight. The reaction was cooled, filtered, and purified by reverse phase HPLC to give the desired product. Yield 8.5 mg. MS: 533.2 (M+H⁺); H¹ NMR (DMSO-d₆): δ (ppm) 0.54-0.75 (m, 4H), 1.20-1.30 (s, 6H), 1.36-1.44 (s, 3H), 2.79-2.90 (m, 1H), 4.52-4.66 (m, 1H), 7.18-7.31 (m, 5H), 7.75-7.98 (m, 5H), 8.40-8.45 (m, 1H), 12.40-1212.51 (m, 1H).

### Example 39

### (S)-2-[4-(4-Cyclopropylcarbamoyl-phenyl)-thiazol-2-ylcarbamoyl]-2,3-dihydro-indole-1-carboxylic acid benzyl ester (compound 7039)

### (S)-2,3-Dihydro-indole-1,2-dicarboxylic acid 1-benzyl ester

(S)-2,3-Dihydro-1H-indole-2-carboxylic acid (254.4 mg, 1.6 mmol) was dissolved in DMF (15 mL) and distilled water (6 mL). The solution was cooled to 0° C., and DIPEA (400 μL, 2.3 mmol) was added followed by benzyl chloroformate (0.330 mL, 2.3 mmol). The reaction was stirred at 0° C. and allowed to warm to ambient temperature overnight. The reaction was filtered and purified by reverse phase HPLC to give the desired product.

### (S)-2-[4-(4-Cyclopropylcarbamoyl-phenyl)-thiazol-2-ylcarbamoyl]-2,3-dihydro-indole-1-carboxylic acid benzyl ester (compound 7039)

(S)-2,3-Dihydro-indole-1,2-dicarboxylic acid 1-benzyl ester (50.5 mg, 0.17 mmol) was dissolved in DMF (1.5 mL). Triethylamine (47 μL, 0.34 mmol) and pentafluorophenyl trifluoroacetate (29 μL, 0.17 mmol) were added, and the reaction was stirred at ambient temperature for 30 minutes. Then 4-(2-amino-thiazol-4-yl)-N-cyclopropyl-benzamide trifluoroacetic acid salt (62.9 mg, 0.17 mmol) was added, and the reaction was stirred at ambient temperature overnight. The reaction was then heated to 70° C. for 4 hours. The reaction was cooled, filtered, and purified by reverse phase HPLC to give the desired product. Yield 16.8 mg. MS: 539.2 (M+H⁺); H¹ NMR (DMSO-d₆): δ (ppm) 0.0.53-0.74 (m, 4H), 2.80-2.89 (m, 1H), 5.01-5.35 (m, 3H), 6.92-7.52 (m, 8H), 7.74-7.99 (m, 6H), 8.41-8.49 (m, 1H), 12.60-12.77 (s, 1H).

### Example 40

### (S)-2-[4-(4-Cyclopropylcarbamoyl-phenyl)-thiazol-2-ylcarbamoyl]-4-methylene-pyrrolidine-1-carboxylic acid tert-butyl ester (Compound 7040)

(S)-4-Methylene-pyrrolidine-1,2-dicarboxylic acid 1-tert-butyl ester (48.5 mg, 0.21 mmol) was dissolved in DMF (2 mL). DIPEA (74.5 μL, 0.43 mmol) was added followed by HATU (81.1 mg, 0.21 mmol). The reaction was stirred at ambient temperature for 15 minutes. Then 4-(2-amino-thiazol-4-yl)-N-cyclopropyl-benzamide (Example 1, 55.5 mg, 0.21 mmol) was added, and the reaction was stirred at ambient temperature overnight. The reaction was then heated to 50° C. overnight. The reaction was cooled, filtered, and purified by reverse phase HPLC to give the desired product. Yield 9.5 mg. MS: 469.1 (M+H⁺); H¹ NMR (DMSO-d₆): δ (ppm) 0.52-0.74 (m, 4H), 1.20-1.30 (s, 6H), 1.37-1.44 (s, 3H), 2.79-2.90 (m, 1H), 2.90-3.08 (m, 1H), 3.89-4.08 (m, 2H), 4.94-5.05 (m, 2H), 7.75-7.92 (s, 1H), 7.82-7.97 (m, 4H), 8.41-8.47 (m, 1H), 12.45-12.58 (m, 1H).

### Example 41

### 4-[4-(4-tert-Butyl-carbamoyl-phenyl)-thiazol-2-ylcarbamoyl]-2-pyridin-4-yl-thiazolidine-3-carboxylic acid benzyl ester (Compound 7041)

From 230 mg of 4-(2-Amino-thiazol-4-yl)-N-tert-butyl-benzamide following procedure for Example 59. MS: 602.2 (M+H⁺); H¹ NMR (DMSO-d₆): δ (ppm) (HCl salt) 12.94 (m, 1H), 8.84 (m, 2H), 8.28 (d, 1H), 7.89-8.09 (m, 7H), 6.82-7.32 (m, 6H), 6.49 (s, 1H), 6.31 (d, 2H), 5.32 (d, 1H), 5.14 (m, 2H), 3.45 (m, 2H), 3.23 (m, 2H), 1.38 (s, 9H).

### Example 42

### (R)-4-[4-(4-Cyclopropylcarbamoyl-phenyl)-thiazol-2-ylcarbamoyl]-2-piperidin-4-yl-thiazolidine-3-carboxylic acid benzyl ester (compound 7042)

### 4-((R)-4-Carboxy-thiazolidin-2-yl)-piperidine-1-carboxylic acid tert-butyl ester

To a solution of L-cysteine hydrochloride (399.8 mg, 2.54 mmol) in distilled water (4 mL), potassium acetate (284.2 mg, 2.90 mmol) was added. Once the solids went into solution, methanol (4 mL) was added followed by 4-formyl-piperidine-1-carboxylic acid tert-butyl ester (653.0 mg, 3.06 mmol). Precipitate crashed out of solution within 30 minutes. The solvent was removed to give the crude product.

### (R)-2-(1-tert-Butoxycarbonyl-piperidin-4-yl)-thiazolidine-3,4-dicarboxylic acid 3-benzyl ester

To a solution of phenyl-methanol (103 μL, 1.00 mmol) in acetonitrile (3 mL), triethylamine (278 μL, 1.99 mmol) was added followed by carbonic acid bis-(2,5-dioxo-pyrrolidin-1-yl) ester (256 mg, 1.00 mmol). The reaction was stirred at ambient temperature for one hour. The solvent was removed, and the residue was redissolved in dry dichloromethane (3 mL). Then triethylamine (139 μL, 1.00 mmol), DMAP (5 mg), and 4-((R)-4-carboxy-thiazolidin-2-yl)-piperidine-1-carboxylic acid tert-butyl ester (211 mg, 0.67 mmol) were added. The reaction was stirred at ambient temperature for 5 hours. The reaction was filtered and purified by reverse phase HPLC to give a diastereomeric mixture of the desired product.

### 4-{(R)-3-Benzyloxycarbonyl-4-[4-(4-cyclopropylcarbamoyl-phenyl)-thiazol-2-ylcarbamoyl]-thiazolidin-2-yl}-piperidine-1-carboxylic acid tert-butyl ester

(R)-2-(1-tert-Butoxycarbonyl-piperidin-4-yl)-thiazolidine-3,4-dicarboxylic acid 3-benzyl ester (170.5 mg, 0.38 mmol) was dissolved in DMF (2.5 mL). DIPEA (132 μL, 0.76 mmol) was added followed by HATU (144 mg, 0.38 mmol). The reaction was stirred at ambient temperature for 15 minutes. Then 4-(2-amino-thiazol-4-yl)-N-cyclopropyl-benzamide (Example 1, 98 mg, 0.38 mmol) was added, and the reaction was stirred at ambient temperature for 6 hours. The reaction was then heated to 50° C. overnight. The solvent was removed, and no further purification steps were taken.

### (R)-4-[4-(4-Cyclopropylcarbamoyl-phenyl)-thiazol-2-ylcarbamoyl]-2-piperidin-4-yl-thiazolidine-3-carboxylic acid benzyl ester (compound 7042)

A solution of 4-{(R)-3-benzyloxycarbonyl-4-[4-(4-cyclopropylcarbamoyl-phenyl)-thiazol-2-ylcarbamoyl]-thiazolidin-2-yl}-piperidine-1-carboxylic acid tert-butyl ester (262 mg, 0.38 mmol) in dichloromethane (1 mL) and trifluoroacetic acid (1 mL) was stirred at ambient temperature for 1 hour. The reaction was filtered and purified by reverse phase HPLC to give the desired product. Yield 13.6 mg. MS: 592.2 (M+H⁺); H¹ NMR (DMSO-d₆): δ (ppm) 0.45-0.78 (m, 4H), 1.12-1.65 (m, 2H), 1.90-2.18 (m, 3H), 2.68-3.00 (m, 3H), 3.00-3.59 (m, 4H), 4.77-5.21 (m, 4H), 7.01-7.24 (m, 3H), 7.26-7.44 (m, 2H), 7.80-8.02 (m, 4H), 8.10-8.30 (m, 1H), 8.43-8.65 (m, 2H).

### Example 43

### (2S,3aS,7aS)-2-[4-(4-Cyclopropylcarbamoyl-phenyl)-thiazol-2-ylcarbamoyl]-octahydro-indole-1-carboxylic acid benzyl ester (Compound 7043)

### (2S,3aS,7aS)-Octahydro-indole-1,2-dicarboxylic acid 1-benzyl ester

(2S,3aS,7aS)-Octahydro-indole-2-carboxylic acid (257.8 mg, 1.52 mmol) was dissolved in DMF (8 mL) and distilled water (8 mL). The solution was cooled to 0° C., and DIPEA (400 μL, 2.30 mmol) was added followed by benzyl chloroformate (325 μL, 2.28 mmol). The reaction was stirred at 0° C. and allowed to warm to ambient temperature overnight. The reaction was filtered and purified by reverse phase HPLC to give the desired product.

### (2S,3aS,7aS)-2-[4-(4-Cyclopropylcarbamoyl-phenyl)-thiazol-2-ylcarbamoyl]-octahydro-indole-1-carboxylic acid benzyl ester (Compound 7043)

(2S,3aS,7aS)-Octahydro-indole-1,2-dicarboxylic acid 1-benzyl (55.9 mg, 0.18 mmol) was dissolved in DMF (2 mL). DIPEA (64 μL, 0.37 mmol) was added followed by HATU (70.7 mg, 0.19 mmol). The reaction was stirred at ambient temperature for 15 minutes. Then 4-(2-amino-thiazol-4-yl)-N-cyclopropyl-benzamide (Example 1, 47.2 mg, 0.18 mmol) was added, and the reaction was stirred at ambient temperature for 6 hours. The reaction was then heated to 45° C. overnight. The reaction was cooled, filtered, and purified by reverse phase HPLC to give the desired product. Yield 13.1 mg. MS: 545.2 (M+H⁺); H¹ NMR (DMSO-d₆): δ (ppm) 0.52-0.75 (m, 4H), 1.35-1.78 (m, 2H), 1.80-2.45 (m, 1H), 2.80-2.90 (m, 1H), 3.75-3.85 (m, 1H), 4.40-4.59 (m, 1H), 4.81-5.08 (m, 2H), 6.97-7.15 (m, 2H), 7.27-7.41 (m, 2H), 7.76-7.99 (m, 5H), 8.41-8.47 (d, 1H), 12.45-12.55 (s, 1H).

### Example 44

### (R)-4-[4-(4-Cyclopropylcarbamoyl-phenyl)-thiazol-2-ylcarbamoyl]-2-phenyl-thiazolidine-3-carboxylic acid benzyl ester (compound 7044)

### (R)-2-Phenyl-thiazolidine-3,4-dicarboxylic acid 3-benzyl ester

(R)-2-Phenyl-thiazolidine-4-carboxylic acid (395.3 mg, 1.89 mmol) was dissolved in DMF (10 mL) and distilled water (10 mL). The solution was cooled to 0° C., and DIPEA (700 μL, 4.02 mmol) was added followed by benzyl chloroformate (410 μL, 2.87 mmol). The reaction was stirred at 0° C. and allowed to warm to ambient temperature overnight. The reaction was filtered and purified by reverse phase HPLC to give the desired product.

### (R)-4-[4-(4-Cyclopropylcarbamoyl-phenyl)-thiazol-2-ylcarbamoyl]-2-phenyl-thiazolidine-3-carboxylic acid benzyl ester (compound 7044)

(R)-2-Phenyl-thiazolidine-3,4-dicarboxylic acid 3-benzyl ester (58.9 mg, 0.17 mmol) was dissolved in DMF (2 mL). DIPEA (60 μL, 0.34 mmol) was added followed by HATU (65.2 mg, 0.17 mmol). The reaction was stirred at ambient temperature for 15 minutes. Then 4-(2-amino-thiazol-4-yl)-N-cyclopropyl-benzamide (Example 1, 44.9 mg, 0.17 mmol) was added, and the reaction was stirred at ambient temperature overnight. The reaction was then heated to 50° C. for 7 hours. The reaction was cooled, filtered, and purified by reverse phase HPLC to give the desired product. Yield 22.8 mg. MS: 585.1 (M+H⁺); H¹ NMR (DMSO-d₆): δ (ppm) 0.54-0.76 (m, 4H), 2.76-2.93 (m, 1H), 3.10-3.24 (m, 1H), 3.43-3.57 (m, 1H), 4.88-5.10 (m, 3H), 6.26-6.29 (s, 1H), 6.91-7.40 (m, 8H), 7.72-8.00 (m, 7H), 8.41-8.48 (d, 1H), 12.69-12.78 (s, 1H).

### Example 45

### (2S,5R)-2-[4-(4-Cyclopropylcarbamoyl-phenyl)-thiazol-2-ylcarbamoyl]-5-(4-morpholin-4-ylmethyl-phenyl)-pyrrolidine-1-carboxylic acid benzyl ester (Compound 7045)

### (S)-2-tert-Butoxycarbonylamino-5-(4-morpholin-4-ylmethyl-phenyl)-5-oxo-pentanoic acid ethyl ester

(S)-5-Oxo-pyrrolidine-1,2-dicarboxylic acid 1-tert-butyl ester 2-ethyl ester (257 mg, 1 mmol) was dissolved in THF (10 mL) and cooled to 0° C. The solution was then treated with a solution of 4-[(4-morpholino)methyl]phenylmagnesium bromide (1 eq, 4 mL of a 0.25 M solution in THF). After addition, the reaction was warmed to room temperature, stirred for 1 hour then quenched with water, the organic solvents removed and the crude product was redissolved in 5 ml of 90% DMF, 10% water with 0.1% TFA and purified by reverse phase HPLC to give the product. MS: 435 (M+H⁺)

### (S)-5-(4-Morpholin-4-ylmethyl-phenyl)-3,4-dihydro-2H-pyrrole-2-carboxylic acid

(S)-2-tert-Butoxycarbonylamino-5-(4-morpholin-4-ylmethyl-phenyl)-5-oxo-pentanoic acid ethyl ester (500 mg, 1.2 mmol) was dissolved in 6 M HCl (10 mL) and heated to 45° C. overnight. The solution was then cooled and the solvents removed under vacuum. The crude product was redissolved in 5 ml of 90% DMF, 10% water with 0.1% TFA and purified by reverse phase HPLC to give the product. MS: 289 (M+H⁺).

### (2S,5R)-5-(4-Morpholin-4-ylmethyl-phenyl)-pyrrolidine-2-carboxylic acid

(S)-5-(4-Morpholin-4-ylmethyl-phenyl)-3,4-dihydro-2H-pyrrole-2-carboxylic acid (121 mg) was dissolved in ethanol (50 mL) with platinum oxide (2 mg) and hydrogenated at 30 psi for 2 hours. The solution was filtered, the solvents removed and used directly in the next reaction. MS: 291 (M+H⁺)

### (2S,5R)-5-(4-Morpholin-4-ylmethyl-phenyl)-pyrrolidine-1,2-dicarboxylic acid 1-benzyl ester

(2S,5R)-5-(4-Morpholin-4-ylmethyl-phenyl)-pyrrolidine-2-carboxylic acid (240 mg, 0.8 mmol) was dissolved in dichloromethane (4 mL) and treated with triethylamine (1 mL) then carbonic acid benzyl ester 2,5-dioxo-pyrrolidin-1-yl ester (1.3 mmol) and DMAP (10 mg). The solution was then stirred at room temperature for 90 minutes. The solvents were removed and the crude product was redissolved in 5 ml of 90% DMF, 10% water with 0.1% TFA and purified by reverse phase HPLC to give the product. MS: 425 (M+H⁺).

### (2S,5R)-2-[4-(4-Cyclopropylcarbamoyl-phenyl)-thiazol-2-ylcarbamoyl]-5-(4-morpholin-4-ylmethyl-phenyl)-pyrrolidine-1-carboxylic acid benzyl ester (Compound 7045)

A solution of (2S,5R)-5-(4-morpholin-4-ylmethyl-phenyl)-pyrrolidine-1,2-dicarboxylic acid 1-benzyl ester (28 mg, 0.07 mmol) in DMF (5 mL) and treated with HATU (1.1 eq. 29 mg) and DIPEA (2.2 eq, 34 μL) and stirred for 15 minutes. Then 1-[4-(2-amino-thiazol-4-yl)-phenyl]-ethanone (1 eq, 20 mg) was added and the mixture stirred at 45° C. overnight. The reaction was cooled, filtered and the solvents removed. The resulting mixture was redissolved in 5 ml of 90% DMF, 10% water with 0.1% TFA and purified by reverse phase HPLC to give the product. Yield 17.3 mg. MS: 666.5 (M+H⁺); H¹-NMR (DMSO-d₆): δ (ppm) 12.6 (s, 1H), 8.5 (m, 1H), 7.9-6.9 (m, 14H), 5.0 (m, 4H), 4.0-3.7 (m, 8H), 3.2-3.0 (m, 8H), 2.5-2.2 (m, 4H), 2.0-1.7 (m, 4H), 1.3-0.9 (m, 4H), 0.7-0.6 (m, 4H).

### Example 46

### (S)-4-[4-(4-Cyclopropylcarbamoyl-phenyl)-thiazol-2-ylcarbamoyl]-2-phenyl-oxazolidine-3-carboxylic acid benzyl ester (Compound 7046)

### 2-Benzyloxycarbonylamino-3-hydroxy-propionic acid methyl ester

To a mixture of L-serine methyl ester hydrochloride (1.88 g, 12.0 mmol) in EtOAc (45 mL) at 0° C. was added to a saturated solution of NaHCO₃ (24 mL) followed by addition of benzylchloroformate (2.22 mL, 15.6 mL). The mixture was allowed to warm to room temperature and stirred for 2 h. The separated organic phase was dried (Na₂SO₄), and concentrated in vacuo to give the crude product. Purification by silica gel furnished (2.41 g, 79% yield) of 2-Benzyloxycarbonylamino-3-hydroxy-propionic acid methyl ester; MS: 254.1 (M+H⁺).

### 2-Phenyl-oxazolidine-3,4-dicarboxylic acid 3-benzyl ester

A mixture of 2-benzyloxycarbonylamino-3-hydroxy-propionic acid methyl ester (0.261 g, 1.03 mmol), benzaldehyde dimethylacetal (0.5 mL, 3.23 mmol), and TsOH (9 mg) in toluene (6 mL) was heated to reflux for 2 h. using a Dean Stark trap. The mixture was concentrated to about 1 mL then diluted with EtOAc (20 mL) and was washed with sat. NaHCO₃, brine, dried (Na₂SO₄), and concentrated in vacuo to give the crude product. Purification by silica gel gave (0.310 g, 88%) of 2-phenyl-oxazolidine-3,4-dicarboxylic acid 3-benzyl ester; MS: 342.1 (M+H⁺).

### 2-Benzyloxycarbonylamino-3-hydroxy-propionic acid

A mixture of 2-phenyl-oxazolidine-3,4-dicarboxylic acid 3-benzyl ester (0.25 g, 0.73 mmol) and aqueous NaOH (1 M, 4 mL, 4 mmol) in THF/H₂O/MeOH (2:2:1, 5 mL) was stirred at room temperature for 3 h. to provide 2-benzyloxycarbonylamino-3-hydroxy-propionic acid; 342.1 (M+H⁺).

### (S)-4-[4-(4-Cyclopropylcarbamoyl-phenyl)-thiazol-2-ylcarbamoyl]-2-phenyl-oxazolidine-3-carboxylic acid benzyl ester (Compound 7046)

A mixture of 2-Benzyloxycarbonylamino-3-hydroxy-propionic acid (0.134 g, 0.41 mmol), HATU (0.16 g, 0.41 mmol), DIPEA (0.11 mL, 0.82 mmol) in DMF (6.0 mL) was stirred at room temperature for 1 h. 4-(2-Amino-thiazol-4-yl)-N-cyclopropyl-benzamide (Example 1, 0.11 g, 0.41 mmol) was added and the reaction mixture was stirred at 50° C. overnight. Purification of the crude product by reverse phase HPLC furnished the desired product. ¹HNMR (DMSO-d₆) δ (Ppm) 12.67 (s, 1H), 8.46-8.44 (d, 1H), 7.97-7.73 (m, 6H), 7.40-7.17 (m, 7H), 6.84 (m, 1H), 6.02 (s, 1H), 5.02-4.87 (m, 3H), 4.35-4.23 (m, 2H), 2.88-2.82 (m, 1H), 0.73-0.55, (m, 4H); MS: 569.2 (M+H⁺).

### Example 47

### (R)-4-[4-(4-Cyclopropylcarbamoyl-phenyl)-thiazol-2-ylcarbamoyl]-2-(2-methoxy-phenyl)-thiazolidine-3-carboxylic acid benzyl ester (Compound 7047)

### (R)-2-(2-Methoxy-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 2-methoxy-benzaldehyde (415 mg, 3.05 mmol). The reaction was stirred at ambient temperature overnight. The solvent was removed, and no further purification steps were taken.

### (R)-2-(2-Methoxy-phenyl)-thiazolidine-3,4-dicarboxylic acid 3-benzyl ester

(R)-2-(2-Methoxy-phenyl)-thiazolidine-4-carboxylic acid (607.3 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(2-methoxy-phenyl)-thiazolidine-3-carboxylic acid benzyl ester (Compound 7047)

(R)-2-(2-Methoxy-phenyl)-thiazolidine-3,4-dicarboxylic acid 3-benzyl ester (91.5 mg, 0.25 mmol) was dissolved in DMF (1.5 mL). DIPEA (85 μL, 0.49 mmol) was added followed by HATU (93.2 mg, 0.25 mmol). The reaction was stirred at ambient temperature for 15 minutes. Then 4-(2-amino-thiazol-4-yl)-N-cyclopropyl-benzamide (Example 1, 63.8 mg, 0.25 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 11.4 mg. MS: 615.1 (M+H⁺); H¹ NMR (DMSO-d₆): δ (ppm) 0.52-0.76 (m, 4H), 2.78-2.90 (m, 1H), 3.01-3.13 (m, 1H), 4.80-5.10 (m, 3H), 6.22-6.37 (d, 1H), 6.90-7.37 (m, 8H), 7.80-8.01 (m, 5H), 8.15-8.22 (m, 1H), 8.40-8.48 (m, 1H), 12.70-12.78 (m, 1H).

### Example 48

### (R)-4-[4-(4-Cyclopropylcarbamoyl-phenyl)-thiazol-2-ylcarbamoyl]-2-(3-fluoro-phenyl)-thiazolidine-3-carboxylic acid benzyl ester (Compound 7048)

### (R)-2-(3-Fluoro-phenyl)-thiazolidine-4-carboxylic acid

To a solution of L-cysteine hydrochloride (400 mg, 2.54 mmol) in distilled water (4 mL), potassium acetate (274 mg, 2.79 mmol) was added. Once the solids went into solution, methanol (4 mL) was added followed by 3-fluoro-benzaldehyde (325 μL, 3.06 mmol). The reaction was stirred at ambient temperature overnight. The solvent was removed, and no further purification steps were taken.

### (R)-2-(3-Fluoro-phenyl)-thiazolidine-3,4-dicarboxylic acid 3-benzyl ester

(R)-2-(3-Fluoro-phenyl)-thiazolidine-4-carboxylic acid (576.7 mg, 2.54 mmol) was dissolved in DMF (12 mL). The solution was cooled to 0° C., and DIPEA (665 μL, 3.82 mmol) was added followed by benzyl chloroformate (545 μL, 3.82 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(3-fluoro-phenyl)-thiazolidine-3-carboxylic acid benzyl ester (Compound 7048)

(R)-2-(3-Fluoro-phenyl)-thiazolidine-3,4-dicarboxylic acid 3-benzyl ester (90.4 mg, 0.25 mmol) was dissolved in DMF (1.5 mL). DIPEA (87 μL, 0.50 mmol) was added followed by HATU (94.7 mg, 0.25 mmol). The reaction was stirred at ambient temperature for 15 minutes. Then 4-(2-amino-thiazol-4-yl)-N-cyclopropyl-benzamide (Example 1, 64.7 mg, 0.25 mmol) was added, and the reaction was stirred at ambient temperature for 5 hours. The reaction was then heated to 50° C. overnight. The reaction was cooled, filtered, and purified by reverse phase HPLC to give a diastereomeric mixture of the desired product. Yield 14.1 mg. MS: 603.1 (M+H⁺); H¹ NMR (DMSO-d₆): δ (ppm) 0.43-0.73 (m, 4H), 2.70-2.88 (m, 1H), 3.05-3.18 (m, 1H), 4.81-5.08 (m, 3H), 6.24 (s, 1H), 6.88-7.55 (m, 8H), 7.62-7.71 (m, 1H), 7.78-7.88 (m, 5H), 8.36-8.43 (m, 1H).

### Example 49

### (R)-4-[4-(4-Cyclopropylcarbamoyl-phenyl)-thiazol-2-ylcarbamoyl]-2-(2-hydroxy-pyridin-4-yl)-thiazolidine-3-carboxylic acid benzyl ester (Compound 7049)

### (R)-2-(2-Hydroxy-pyridin-4-yl)-thiazolidine-4-carboxylic acid

To a solution of L-cysteine hydrochloride (402.9 mg, 2.56 mmol) in distilled water (4 mL), potassium acetate (281.7 mg, 2.87 mmol) was added. Once the solids went into solution, methanol (4 mL) was added followed by 2-hydroxy-pyridine-4-carbaldehyde (376.4 mg, 3.06 mmol). Precipitate crashed out of solution within one hour, and the reaction was stirred at ambient temperature overnight. The solvent was removed to give the crude product.

### (R)-2-(2-Hydroxy-pyridin-4-yl)-thiazolidine-3,4-dicarboxylic acid 3-benzyl ester

(R)-2-(2-Hydroxy-pyridin-4-yl)-thiazolidine-4-carboxylic acid (578 mg, 2.56 mmol) was dissolved in DMF (12 mL). The solution was cooled to 0° C., and DIPEA (665 μL, 3.82 mmol) was added followed by benzyl chloroformate (905 μL, 6.34 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(2-hydroxy-pyridin-4-yl)-thiazolidine-3-carboxylic acid benzyl ester (Compound 7049)

(R)-2-(2-Hydroxy-pyridin-4-yl)-thiazolidine-3,4-dicarboxylic acid 3-benzyl ester (83.7 mg, 0.23 mmol) was dissolved in DMF (1.5 mL). DIPEA (81 μL, 0.47 mmol) was added followed by HATU (89.1 mg, 0.23 mmol). The reaction was stirred at ambient temperature for 15 minutes. Then 4-(2-amino-thiazol-4-yl)-N-cyclopropyl-benzamide (Example 1, 60.6 mg, 0.23 mmol) was added, and the reaction was stirred at ambient temperature for 4 hours. The reaction was then heated to 50° C. overnight. The reaction was cooled, filtered, and purified by reverse phase HPLC to give a diastereomeric mixture of the desired product. Yield 14.9 mg. MS: 602.1 (M+H⁺); H¹ NMR (DMSO-d₆): δ (ppm) 0.49-0.75 (m, 4H), 2.78-2.91 (m, 1H), 3.02-3.19 (m, 1H), 4.79-5.29 (m, 3H), 5.95-6.09 (m, 1H), 6.35-6.49 (m, 1H), 6.84-7.45 (m, 9H), 7.80-8.04 (m, 4H), 8.42-8.51 (d, 1H).

### Example 50

### (R)-4-[4-(4-Cyclopropylcarbamoyl-phenyl)-thiazol-2-ylcarbamoyl]-2-(1-oxy-pyridin-4-yl)-thiazolidine-3-carboxylic acid benzyl ester (Compound 7050)

### (R)-2-(1-Oxy-pyridin-4-yl)-thiazolidine-4-carboxylic acid

To a solution of L-cysteine hydrochloride (399.1 mg, 2.53 mmol) in distilled water (4 mL), potassium acetate (281.0 mg, 2.86 mmol) was added. Once the solids went into solution, methanol (4 mL) was added followed by 1-oxy-pyridine-4-carbaldehyde (376.8 mg, 3.06 mmol). Precipitate crashed out of solution within one hour, and the reaction was stirred at ambient temperature overnight. The solvent was removed to give the crude product.

### (R)-2-(1-Oxy-pyridin-4-yl)-thiazolidine-3,4-dicarboxylic acid 3-benzyl ester

(R)-2-(1-Oxy-pyridin-4-yl)-thiazolidine-4-carboxylic acid (573 mg, 2.53 mmol) was dissolved in DMF (12 mL). The solution was cooled to 0° C., and DIPEA (665 μL, 3.82 mmol) was added followed by benzyl chloroformate (905 μL, 6.34 mmol). The reaction was stirred at 0° C. and allowed to warm to ambient temperature over 5 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(1-oxy-pyridin-4-yl)-thiazolidine-3-carboxylic acid benzyl ester (Compound 7050)

(R)-2-(1-Oxy-pyridin-4-yl)-thiazolidine-3,4-dicarboxylic acid 3-benzyl ester (84 mg, 0.23 mmol) was dissolved in DMF (1.5 mL). DIPEA (81 μL, 0.47 mmol) was added followed by HATU (89 mg, 0.23 mmol). The reaction was stirred at ambient temperature for 15 minutes. Then 4-(2-amino-thiazol-4-yl)-N-cyclopropyl-benzamide (Example 1, 61 mg, 0.24 mmol) was added, and the reaction was stirred at ambient temperature for 4 hours. The reaction was then heated to 50° C. overnight. The reaction was cooled, filtered, and purified by reverse phase HPLC to give a diastereomeric mixture of the desired product. Yield 15.4 mg. MS: 602.2 (M+H⁺); H¹ NMR (DMSO-d₆): δ (ppm) 0.52-0.76 (m, 4H), 2.79-2.82 (m, 1H), 3.12-3.25 (m, 1H), 3.50-3.61 (m, 1H), 4.88-5.21 (m, 3H), 6.30 (s, 1H), 6.98-7.38 (m, 5H), 7.78-8.05 (m, 6H), 8.10-8.35 (m, 2H), 8.42-8.50 (d, 1H).

### Example 51

### (R)-4-[4-(4-Cyclopropylcarbamoyl-phenyl)-thiazol-2-ylcarbamoyl]-2-(4-methoxycarbonyl-phenyl)-thiazolidine-3-carboxylic acid benzyl ester (Compound 7051)

### (R)-2-(4-Methoxycarbonyl-phenyl)-thiazolidine-4-carboxylic acid

To a solution of L-cysteine hydrochloride monohydrate (307.1 mg, 1.75 mmol) in distilled water (2.6 mL), potassium acetate (184.7 mg, 1.88 mmol) was added. Once the solids went into solution, methanol (2.6 mL) was added followed by 4-formyl-benzoic acid methyl ester (346.9 mg, 2.11 mmol). The reaction was stirred at ambient temperature, and precipitate crashed out of solution within 15 minutes. The solvent was removed to give the crude product.

### (R)-2-(4-Methoxycarbonyl-phenyl)-thiazolidine-3,4-dicarboxylic acid 3-benzyl ester

(R)-2-(4-Methoxycarbonyl-phenyl)-thiazolidine-4-carboxylic acid (467 mg, 1.75 mmol) was dissolved in DMF (15 mL). The solution was cooled to 0° C., and DIPEA (455 μL, 2.61 mmol) was added followed by benzyl chloroformate (375 μL, 2.63 mmol). The reaction was stirred at 0° C. and allowed to warm to ambient temperature overnight. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(4-methoxycarbonyl-phenyl)-thiazolidine-3-carboxylic acid benzyl ester (Compound 7051)

(R)-2-(4-Methoxycarbonyl-phenyl)-thiazolidine-3,4-dicarboxylic acid 3-benzyl ester (276.7 mg, 0.70 mmol) was dissolved in DMF (4 mL). DIPEA (240 μL, 1.38 mmol) was added followed by HATU (263.3, 0.69 mmol). The reaction was stirred at ambient temperature for 15 minutes. Then 4-(2-amino-thiazol-4-yl)-N-cyclopropyl-benzamide (Example 1, 179.3 mg, 0.69 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. Yield 5.8 mg. MS: 643.3 (M+H⁺); H¹ NMR (DMSO-d₆): δ (ppm) 0.52-0.75 (m, 4H), 2.78-2.90 (m, 1H), 3.08-3.22 (m, 1H), 3.78-3.88 (s, 3H), 4.88-5.11 (m, 3H), 6.30-6.38 (s, 1H), 6.89-7.25 (m, 5H), 7.78-7.99 (m, 9H), 8.40-8.49 (d, 1H), 12.70-12.79 (s, 1H).

### Example 52

### (2S,5R)-2-[4-(4-Cyclopropylcarbamoyl-phenyl)-thiazol-2-ylcarbamoyl]-5-phenyl-pyrrolidine-1-carboxylic acid benzyl ester (compound 7052)

### (2S,5R)-5-Phenyl-pyrrolidine-2-carboxylic acid [4-(4-cyclopropylcarbamoyl-phenyl)-thiazol-2-yl]-amide

A solution of (2S,5R)-2-[4-(4-cyclopropylcarbamoyl-phenyl)-thiazol-2-ylcarbamoyl]-5-phenyl-pyrrolidine-1-carboxylic acid tert-butyl ester (Example 5, 271.5 mg, 0.51 mmol) in dichloromethane (2 mL) and trifluoroacetic acid (2 mL) was stirred at ambient temperature overnight. The reaction was filtered and purified by reverse phase HPLC to give the desired product.

### (2S,5R)-2-[4-(4-Cyclopropylcarbamoyl-phenyl)-thiazol-2-ylcarbamoyl]-5-phenyl-pyrrolidine-1-carboxylic acid benzyl ester (compound 7052)

(2S,5R)-5-Phenyl-pyrrolidine-2-carboxylic acid [4-(4-cyclopropylcarbamoyl-phenyl)-thiazol-2-yl]-amide (259.5 mg, 0.60 mmol) was dissolved in DMF (3 mL). The solution was cooled to 0° C., and DIPEA (157 μL, 0.90 mmol) was added followed by benzyl chloroformate (128 μL, 0.90 mmol). The reaction was stirred at 0° C. and allowed to warm to ambient temperature overnight. The reaction was filtered and purified by reverse phase HPLC to give the desired product. Yield 103.9 mg. MS: 567.2 (M+H⁺); H¹ NMR (DMSO-d₆): δ (ppm) 0.53-0.75 (m, 4H), 1.78-2.03 (m, 2H), 2.19-2.46 (m, 2H), 2.80-2.90 (m, 1H), 5.10-5.23 (m, 1H), 5.32-5.17 (m, 4H), 6.78-6.88 (m, 1H), 7.00-7.40 (m, 7H), 7.61-7.70 (m, 2H), 7.78-8.01 (m, 5H), 8.42-8.49 (d, 1H), 12.55-12.64 (s, 1H).

### Example 53

### (R)-4-[4-(4-Cyclopropylcarbamoyl-phenyl)-thiazol-2-ylcarbamoyl]-2-(1-methyl-1H-imidazol-2-yl)-thiazolidine-3-carboxylic acid benzyl ester (Compound 7053)

### (R)-2-(1-Methyl-1H-imidazol-2-yl)-thiazolidine-4-carboxylic acid

To a solution of L-cysteine hydrochloride monohydrate (305.8 mg, 1.74 mmol) in distilled water (2.6 mL), potassium acetate (188.0 mg, 1.92 mmol) was added. Once the solids went into solution, methanol (2.6 mL) was added followed by 1-methyl-1H-imidazole-2-carbaldehyde (240.7 mg, 2.19 mmol). The reaction was stirred at ambient temperature overnight. The solvent was removed, and no further purification steps were taken.

### 2-(1-Methyl-1H-imidazol-2-yl)-thiazolidine-3,4-dicarboxylic acid 3-benzyl ester

(R)-2-(1-Methyl-1H-imidazol-2-yl)-thiazolidine-4-carboxylic acid (371 mg, 1.74 mmol) was dissolved in DMF (15 mL). The solution was cooled to 0° C., and DIPEA (455 μL, 2.61 mmol) was added followed by benzyl chloroformate (375 μL, 2.63 mmol). The reaction was stirred at 0° C. and allowed to warm to ambient temperature overnight. The reaction was filtered and purified by reverse phase HPLC. One of the diasteromers was able to be isolated as a pure fraction. Both the pure diasteromer and the mixture were subjected to the subsequent coupling conditions.

### (R)-4-[4-(4-Cyclopropylcarbamoyl-phenyl)-thiazol-2-ylcarbamoyl]-2-(1-methyl-1H-imidazol-2-yl)-thiazolidine-3-carboxylic acid benzyl ester (Compound 7053)

The diastereomeric mixture of (R)-2-(1-methyl-1H-imidazol-2-yl)-thiazolidine-3,4-dicarboxylic acid 3-benzyl ester (59.3 mg, 0.17 mmol) was dissolved in DMF (1 mL). DIPEA (59 μL, 0.34 mmol) was added followed by HATU (130 mg, 0.34 mmol). The reaction was stirred at ambient temperature for 15 minutes. Then 4-(2-amino-thiazol-4-yl)-N-cyclopropyl-benzamide (Example 1, 44.4 mg, 0.17 mmol) was added, and the reaction was stirred at ambient temperature for 5 hours. The reaction was then heated to 50° C. overnight. The reaction was cooled, filtered, and purified by reverse phase HPLC to give the desired product. Yield 8.6 mg. MS: 589.2 (M+H⁺); H¹ NMR (DMSO-d₆): δ (ppm) 0.54-0.76 (m, 4H), 2.81-2.90 (m, 1H), 4.77-5.28 (m, 3H), 6.68-6.77 (m, 1H), 7.05-7.45 (m, 6H), 7.77-8.08 (m, 5H), 8.40-8.49 (m, 1H).

### Example 54

### 4-[4-(4-Cyclopropylcarbamoyl-phenyl)-thiazol-2-ylcarbamoyl]-2-(1-methyl-1H-imidazol-2-yl)-thiazolidine-3-carboxylic acid benzyl ester (Compound 7054)

One of the other single diastereomer from Example 53, either (2R,4R)-2-(1-methyl-1H-imidazol-2-yl)-thiazolidine-3,4-dicarboxylic acid 3-benzyl ester or (2S,4R)-2-(1-methyl-1H-imidazol-2-yl)-thiazolidine-3,4-dicarboxylic acid 3-benzyl ester (59.7 mg, 0.17 mmol), was dissolved in DMF (1 mL). DIPEA (59 μL, 0.34 mmol) was added followed by HATU (130 mg, 0.34 mmol). The reaction was stirred at ambient temperature for 15 minutes. Then 4-(2-amino-thiazol-4-yl)-N-cyclopropyl-benzamide (Example 1, 45.2 mg, 0.17 mmol) was added, and the reaction was stirred at ambient temperature for 5 hours. The reaction was then heated to 50° C. overnight. The reaction was cooled, filtered, and purified by reverse phase HPLC to give the desired product. Yield 14.5 mg. MS: 589.2 (M+H⁺); H¹ NMR (DMSO-d₆): δ (ppm) 0.54-0.75 (m, 4H), 2.76-2.93 (m, 1H), 4.75-5.21 (m, 3H), 6.69-6.78 (m, 1H), 7.03-7.45 (m, 6H), 7.78-8.07 (m, 5H), 8.41-8.48 (m, 1H).

### Example 55

### 4-[4-(4-Cyclopropylcarbamoyl-phenyl)-thiazol-2-ylcarbamoyl]-2-pyridin-3-yl-thiazolidine-3-carboxylic acid tert-butyl ester (Compound 7055)

The unidentified single diastereomer, either (2R,4R)-2-pyridin-3-yl-thiazolidine-3,4-dicarboxylic acid 3-tert-butyl ester or (2S,4R)-2-pyridin-3-yl-thiazolidine-3,4-dicarboxylic acid 3-tert-butyl ester (57.6 mg, 0.19 mmol), was dissolved in DMF (1 mL). DIPEA (63 μL, 0.36 mmol) was added followed by HATU (70.0 mg, 0.18 mmol). The reaction was stirred at ambient temperature for 15 minutes. Then 4-(2-amino-thiazol-4-yl)-N-cyclopropyl-benzamide (Example 1, 47.3 mg, 0.18 mmol) was added, and the reaction was stirred at ambient temperature for 5 hours. The reaction was then heated to 50° C. overnight. The reaction was cooled, filtered, and purified by reverse phase HPLC to give the desired product. Yield 5.5 mg. MS: 552.1 (M+H⁺); H¹ NMR (DMSO-d₆): δ (ppm) 0.51-0.75 (m, 4H), 4.76-4.98 (m, 1H), 7.56-7.71 (m, 1H), 7.77-8.00 (m, 6H), 8.32-8.50 (m, 2H), 8.54-8.65 (m, 1H), 8.90-9.07 (m, 1H), 12.68-12.77 (m, 1H).

### Example 56

### (R)-4-[4-(4-Cyclopropylcarbamoyl-phenyl)-thiazol-2-ylcarbamoyl]-2-oxazol-2-yl-thiazolidine-3-carboxylic acid benzyl ester (Example 7056)

### (R)-2-Oxazol-2-yl-thiazolidine-4-carboxylic acid

To a solution of L-cysteine hydrochloride (448.9 mg, 2.85 mmol) in distilled water (4.3 mL), potassium acetate (348.3 mg, 3.55 mmol) was added. Once the solids went into solution, methanol (4.3 mL) was added followed by oxazole-2-carbaldehyde (330.8 mg, 3.41 mmol). The reaction was stirred at ambient temperature for 4 hours. The solvent was removed, and no further purification steps were taken.

### (R)-2-Oxazol-2-yl-thiazolidine-3,4-dicarboxylic acid 3-benzyl ester

(R)-2-Oxazol-2-yl-thiazolidine-4-carboxylic acid (568 mg, 2.84 mmol) was dissolved in DMF (15 mL). The solution was cooled to 0° C., and DIPEA (740 μL, 4.25 mmol) was added followed by benzyl chloroformate (610 μL, 4.27 mmol). The reaction was stirred at 0° C. and allowed to warm to ambient temperature over 3 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-oxazol-2-yl-thiazolidine-3-carboxylic acid benzyl ester (Example 7056)

(R)-2-Oxazol-2-yl-thiazolidine-3,4-dicarboxylic acid 3-benzyl ester (58 mg, 0.17 mmol) was dissolved in DMF (1 mL). DIPEA (61 μL, 0.35 mmol) was added followed by HATU (66 mg, 0.17 mmol). The reaction was stirred at ambient temperature for 15 minutes. Then 4-(2-amino-thiazol-4-yl)-N-cyclopropyl-benzamide (Example 1, 45 mg, 0.17 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. Yield 13.3 mg. MS: 576.3 (M+H⁺); H¹ NMR (DMSO-d₆): δ (ppm) 0.51-0.75 (m, 4H), 1.10-1.29 (m, 2H), 2.78-2.92 (m, 1H), 3.60-3.79 (m, 2H), 4.79-5.28 (m, 5H), 6.47-6.60 (m, 1H), 6.96-7.41 (m, 9H), 7.77-8.70 (m, 7H), 8.40-8.50 (m, 1H).

### Example 57

### (R)-4-[4-(4-Cyclopropylcarbamoyl-phenyl)-thiazol-2-ylcarbamoyl]-2-(3-methoxycarbonyl-phenyl)-thiazolidine-3-carboxylic acid benzyl ester (Compound 7057)

### (R)-2-(3-Methoxycarbonyl-phenyl)-thiazolidine-4-carboxylic acid

To a solution of L-cysteine hydrochloride (452.6 mg, 2.87 mmol) in distilled water (4.3 mL), potassium acetate (348.9 mg, 3.56 mmol) was added. Once the solids went into solution, methanol (4.3 mL) was added followed by 3-formyl-benzoic acid methyl ester (566.7 mg, 3.45 mmol). The reaction was stirred at ambient temperature for 4 hours. The solvent was removed, and no further purification steps were taken.

### (R)-2-(3-Methoxycarbonyl-phenyl)-thiazolidine-3,4-dicarboxylic acid 3-benzyl ester

(R)-2-(3-Methoxycarbonyl-phenyl)-thiazolidine-4-carboxylic acid (768 mg, 2.87 mmol) was dissolved in DMF (15 mL). The solution was cooled to 0° C., and DIPEA (750 μL, 4.31 mmol) was added followed by benzyl chloroformate (615 μL, 4.31 mmol). The reaction was stirred at 0° C. and allowed to warm to ambient temperature over 3 hours. The reaction was filtered and purified by reverse phase HPLC to give a diastereomeric mixture of the desired product.

### (R)-4-[4-(4-Cyclopropylcarbamoyl-phenyl)-thiazol-2-ylcarbamoyl]-2-(3-methoxycarbonyl-phenyl)-thiazolidine-3-carboxylic acid benzyl ester (Compound 7057)

(R)-2-(3-Methoxycarbonyl-phenyl)-thiazolidine-3,4-dicarboxylic acid 3-benzyl ester (187 mg, 0.47 mmol) was dissolved in DMF (3 mL). DIPEA (163 μL, 0.94 mmol) was added followed by HATU (177 mg, 0.47 mmol). The reaction was stirred at ambient temperature for 15 minutes. Then 4-(2-amino-thiazol-4-yl)-N-cyclopropyl-benzamide (Example 1, 121 mg, 0.47 mmol) was added, and the reaction was stirred at ambient temperature overnight. The reaction was then heated to 50° C. overnight. The reaction was cooled, filtered, and purified by reverse phase HPLC to give a diastereomeric mixture of the desired product. Yield 5.1 mg. MS: 643.2 (M+H⁺); H¹ NMR (DMSO-d₆): δ (ppm) 0.52-0.75 (m, 4H), 2.75-2.89 (m, 1H), 3.86 (s, 3H), 6.36 (s, 1H), 6.90-7.24 (m, 5H), 7.47-7.56 (m, 1H), 7.78-7.99 (m, 4H), 8.09-8.17 (m, 1H), 8.24-8.32 (m, 1H), 8.40-8.46 (m, 1H).

### Example 58

### (2R,4R)-4-(4-Phenyl-thiazol-2-ylcarbamoyl)-2-pyridin-3-yl-thiazolidine-3-carboxylic acid benzyl ester (Compound 7058)

(2R,4R)-2-Pyridin-3-yl-thiazolidine-3,4-dicarboxylic acid 3-benzyl ester (68.8 mg, 0.20 mmol) was dissolved in DMF (1 mL). DIPEA (69 μL, 0.40 mmol) was added followed by HATU (75.6 mg, 0.20 mmol). The reaction was stirred at ambient temperature for 15 minutes. Then 4-phenyl-thiazol-2-ylamine (35.5 mg, 0.20 mmol) was added, and the reaction was stirred at ambient temperature for 6 hours. The reaction was then heated to 50° C. overnight. The reaction was cooled, filtered, and purified by reverse phase HPLC to give the desired product. Yield 7.4 mg. MS: 503.1 (M+H⁺); H¹ NMR (DMSO-d₆): δ (ppm) 3.15-3.31 (m, 1H), 4.88-5.15 (m, 3H), 6.42 (s, 1H), 6.95-7.94 (m, 13H), 8.40-8.71 (m, 2H), 9.00-9.13 (m, 1H), 12.77 (s, 1H).

### Example 59

### 4-Phenylcarbamoyl-2-pyridin-4-yl-thiazolidine-3-carboxylic acid benzyl ester (Compound 7059)

### 2-Pyridin-4-yl-thiazolidine-4-carboxylic acid

To a solution of L-cysteine hydrochloride monohydrate (499.0 mg, 2.84 mmol) in distilled water (4.3 mL), potassium acetate (317.3 mg, 3.23 mmol) was added. Once the solids went into solution, methanol (4.3 mL) was added followed by pyridine-4-carbaldehyde (325 μL, 3.45 mmol). The reaction was stirred at ambient temperature for 5 hours. The solvent was removed, and no further purification steps were taken.

### 2-Pyridin-4-yl-thiazolidine-3,4-dicarboxylic acid 3-benzyl ester

2-Pyridin-4-yl-thiazolidine-4-carboxylic acid (500 mg, 2.38 mmol) was dissolved in DMF (15 mL). The solution was cooled to 0° C., and DIPEA (620 μL, 3.56 mmol) was added followed by benzyl chloroformate (510 μL, 3.57 mmol). The reaction was stirred at 0° C. and allowed to warm to ambient temperature overnight. The reaction was filtered and purified by reverse phase HPLC to give a diastereomeric mixture of the desired product.

### 4-Phenylcarbamoyl-2-pyridin-3-yl-thiazolidine-3-carboxylic acid benzyl ester (Compound 7059)

2-Pyridin-4-yl-thiazolidine-3,4-dicarboxylic acid 3-benzyl ester (58.6 mg, 0.17 mmol) was dissolved in DMF (1 mL). DIPEA (59 μL, 0.34 mmol) was added followed by HATU (64.5 mg, 0.17 mmol). The reaction was stirred at ambient temperature for 15 minutes. Then aniline (16 mg, 0.17 mmol) was added, and the reaction was stirred at ambient temperature overnight. The reaction was then heated to 50° C. overnight. The reaction was cooled, filtered, and purified by reverse phase HPLC to give the desired product. MS: 592.7 (M+H⁺); H¹ NMR (DMSO-d₆): δ (ppm) (HCl salt) 10.38 (d, 1H), 8.78 (dd, 2H), 7.99 (dd, 2H), 7.78 (dd, 2H), 7.30 (m, 8H), 6.31 (d, 1H), 5.16 (m, 1H), 4.98 (m, 2H), 3.64 (m, 1H), 3.21 (m, 1H), 1.23 (m, 2H).

### Example 60

### 4-(2-Methyl-cyclohexylcarbamoyl)-2-pyridin-4-yl-thiazolidine-3-carboxylic acid benzyl ester (Compound 7060)

Prepared using experimental procedure described for compound 7031 (Example 31). MS: 440.7 (M+H⁺)

### Example 61

### 2-Pyridin-4-yl-4-(1,2,3,4-tetrahydro-naphthalen-1-ylcarbamoyl)-thiazolidine-3-carboxylic acid benzyl ester (Compound 7061)

Prepared using experimental procedure described for compound 7031 (Example 31). MS: 474.7 (M+H⁺)

### Example 62

### 2-Pyridin-4-yl-4-(thiazol-2-ylcarbamoyl)-thiazolidine-3-carboxylic acid benzyl ester (Compound 7062)

From 20 mg of 2-thiazole amine following procedure for compound 7059 (Example 59). MS: 427.1 (M+H⁺); H¹ NMR (DMSO-d₆): δ (ppm) (HCl salt) 8.86 (d, 2H), 8.08 (dd, 1H), 7.51 (br s, 1H), 7.20 (m, 7H), 6.86 (d, 1H), 5.37 (d, 1H), 5.01 (m, 2H), 3.64 (m, 1H), 3.48 (m, 1H).

### Example 63

### 4-(Indan-2-ylcarbamoyl)-2-pyridin-4-yl-thiazolidine-3-carboxylic acid benzyl ester (Compound 7063)

Prepared using experimental procedure described for compound 7031 (Example 31). MS: 460.7 (M+H⁺)

### Example 64

### 4-[4-(4-Cyclopropylcarbamoyl-phenyl)-thiazol-2-ylcarbamoyl]-2-pyridin-4-yl-oxazolidine-3-carboxylic acid benzyl ester (Compound 7064)

### 4-Dimethoxymethyl-pyridine

A mixture of 4-pyridinecarbaldehyde (3.0 ml, 31.9 mmol), trimethylorthofomate (40 mL), TsOH (1.82 g, 9.57 mmol), and MeOH (20 mL) was heated to reflux overnight, concentrated and then dissolved in EtOAc (50 mL). The mixture was washed with Sat.NaHCO₃, brine, dried (Na₂SO₄), and concentrated to give the crude product. Purification by ISCO gave the desired product 4-dimethoxymethyl-pyridine; MS: 154.1 (M+H⁺).

### 2-Benzyloxycarbonylamino-3-hydroxy-propionic acid methyl ester

To a mixture of L-Serine methyl ester hydrochloride (1.88 g, 12.0 mmol) in ETOAc (45 mL) at 0° C. was added Sat. NaHCO₃ (24 mL) followed by benzylchloroformate (2.22 mL, 15.6 mmol). The mixture was allowed to warm to room temperature and stirred for 2 h. The organic phase was separated, dried (Na₂SO₄), and concentrated to give the crude product. Purification by ISCO gave the desired product 2-benzyloxycarbonylamino-3-hydroxy-propionic acid methyl ester; MS: 255.1 (M+H⁺).

### (S)-2-Pyridin-4-yl-oxazolidine-3,4-dicarboxylic acid 3-benzyl ester 4-methyl ester

A mixture of 2-benzyloxycarbonylamino-3-hydroxy-propionic acid methyl ester, 0.36 g, 1.42 mmol), 4-dimethoxymethylpyridine, 0.7 g, 4.57 mmol), and TsOH (0.27 g, 1.6 mmol) in toluene (20 mL) was heated to reflux for 5 h. The mixture was concentrated, dissolved in EtOAc (30 mL), washed with Sat.NaHCO₃, brine, dried (Na₂SO₄), and concentrated to give the crude product. Purification by ISCO gave the desired product 2-pyridin-4-yl-oxazolidine-3,4-dicarboxylic acid 3-benzyl ester 4-methyl ester; MS: 343.1 (M+H⁺).

### (S)-2-Pyridin-4-yl-oxazolidine-3,4-dicarboxylic acid 3-benzyl ester

A mixture of 2-pyridin-4-yl-oxazolidine-3,4-dicarboxylic acid 3-benzyl ester 4-methyl ester, 0.140 g, 0.41 mmol) and NaOH (1M, 2 mL, 2 mmol) in THF/H₂O/MeOH (2/2/1) was stirred at room temperature for 2 h, acidified with HCl (IN) and concentrated to give the crude product (S)-2-pyridin-4-yl-oxazolidine-3,4-dicarboxylic acid 3-benzyl ester; MS: 329.1.

### 4-[4-(4-Cyclopropylcarbamoyl-phenyl)-thiazol-2-ylcarbamoyl]-2-pyridin-4-yl-oxazolidine-3-carboxylic acid benzyl ester (Compound 7064)

A mixture of (S)-2-pyridin-4-yl-oxazolidine-3,4-dicarboxylic acid 3-benzyl ester, (0.12 g, 0.37 mmol), HATU (0.615 g, 1.70 mmol), and DIEA (0.25 mL, 1.9 mmol) in DMF (6.0 mL) was stirred at room temperature for 1 h., 4-(2-Amino-thiazol-4-yl)-N-cyclopropyl-benzamide, 0160 g, 0.62 mmol) was added and the reaction mixture was heated to 50° C. overnight. The resulting mixture was purified by reverse phase HPLC to furnish the desired product. MS: 570 (M+H⁺); ¹H NMR (DMSO-d₆) δ (ppm) 12.79 (bs, 1H), 8.89 (m, 3H), 8.46-8.05 (m, 3H), 8.04-7.84 (m, 6H), 7.64-7.61 (m, 1H), 7.25-6.98 (m, 7H), 6.45-6.24 (m, 1H), 5.31-4.97 (m, 4H), 4.46-3.30 (m, under water peak), 3.39-3.37 (m, 2H), 2.71 (m, 1H), 1.23-1.11 (m, 10H), 0.68-0.58, (m, 4H).

### Example 65

### 2-(2-Carboxy-ethyl)-4-[4-(4-cyclopropylcarbamoyl-phenyl)-thiazol-2-ylcarbamoyl]-thiazolidine-3-carboxylic acid benzyl ester (Compound 7065)

To a solution of 4-[4-(4-cyclopropylcarbamoyl-phenyl)-thiazol-2-ylcarbamoyl]-2-(2-methoxycarbonyl-ethyl)-thiazolidine-3-carboxylic acid benzyl ester (89.7 mg, 0.15 mmol) in THF:H₂O:MeOH (2:1:1), LiOH (31.5 mg, 0.75 mmol) was added and the reaction was heated to 50° C. overnight. The reaction was cooled, filtered, and purified by reverse phase HPLC to give a diastereomeric mixture of the desired products. MS: 581.1 (M+H⁺); H¹ NMR (DMSO-d₆): δ (ppm) 0.54-0.85 (m, 4H), 1.93-2.45 (m, 4H), 2.80-2.92 (m, 1H), 3.17-3.60 (m, 2H), 4.79-5.30 (m, 4H), 7.04-7.44 (m, 4H), 7.80-8.01 (m, 5H), 8.41-8.50 (m, 1H).

### Example 66

### 4-(4-Cyclopropylcarbamoyl-phenylcarbamoyl)-2-pyridin-4-yl-thiazolidine-3-carboxylic acid benzyl ester (Compound 7066)

This was prepared by coupling 2-pyridin-4-yl-thiazolidine-3,4-dicarboxylic acid 3-benzyl ester and 4-amino-N-cyclopropyl-benzamide using an amide coupling procedure similar to compound 7078 (Example 78). MS: 503.1 (M+H⁺).

### Example 67

### 4-{4-[(3-Phenyl-ureido)-methyl]-phenylcarbamoyl}-2-pyridin-4-yl-thiazolidine-3-carboxylic acid benzyl ester (Compound 7067)

This was prepared using a procedure similar to compound 7124 (Example 124). MS: 568.5 (M+H⁺).

### Example 68

### 4-(4-Cyclopropylcarbamoylmethyl-phenylcarbamoyl)-2-pyridin-4-yl-thiazolidine-3-carboxylic acid benzyl ester (compound 7068)

Prepared using experimental procedure described for compound 7031 (Example 31). MS: 517.7 (M+H⁺)

### Example 69

### 3-(Tetrahydro-furan-2-carbonyl)-2-(tetrahydro-pyran-4-yl)-thiazolidine-4-carboxylic acid [4-(4-cyclopropylcarbamoyl-phenyl)-thiazol-2-yl]-amide (Compound 7069)

Prepared using experimental procedure described for compound 7104 (Example 104). MS: 557.1 (M+H⁺); ¹H NMR (DMSO-d₆): δ (ppm) 12.60 (s, 0.6H), 12.49 (s, 0.3H), 8.45-8.44 (d, 1H), 7.96-7.78 (m, 4H), 5.87-5.37 (m, 1H), 5.34-5.20 (m, 0.4H), 4.91-4.88 (m, 0.7H), 4.85-4.77 (m, 0.55H), 4.74-4.52 (m, 0.4H), 3.91-3.84 (m, 2H), 3.76-3.31 (bs, underwater), 3.54-3.31 (m, 13H), 2.92-2.82 (m, 2H), 2.08-1.7 (m, 8H), 1.35-1.23 (m, 3H), 0.73-0.55 (m, 4H).

### Example 70

### 3-(2,2-Dimethyl-propionyl)-2-(tetrahydro-pyran-4-yl)-thiazolidine-4-carboxylic acid [4-(4-cyclopropylcarbamoyl-phenyl)-thiazol-2-yl]-amide (Compound 7070)

Prepared using experimental procedure described for compound 7104 (Example 104). MS: 543.1 (M+H⁺); ¹H NMR (DMSO-d₆): δ (ppm) 12.55 (s, 0.7H), 8.46-8.44 (d, 2H), 7.97-7.80 (m, 5H), 5.42-5.29 (m, 2H), 3.89-3.86 (m, 3H), 3.51-3.14 (m, under water peak), 2.88-2.82 (m, 1H), 2.03-2.00 (m, 1H), 1.83-1.70 (m, 3H), 1.33-1.10 (m, 14H), 0.73-0.55 (m, 4H).

### Example 71

### 4-[4-(4-Cyclopropylcarbamoyl-phenyl)-thiazol-2-ylcarbamoyl]-2-(5-morpholin-4-yl-5-oxo-pentyl)-thiazolidine-3-carboxylic acid benzyl ester (Compound 7071)

Prepared from 2-(4-carboxy-butyl)-4-[4-(4-cyclopropylcarbamoyl-phenyl)-thiazol-2-ylcarbamoyl]-thiazolidine-3-carboxylic acid benzyl ester and morpholine using experimental procedures described for compound 7125 (Example 125). MS: 678.2 (M+H⁺); H¹ NMR (DMSO-d₆): δ (ppm) 0.54-0.77 (m, 4H), 1.28-2.11 (m, 12H), 2.20-2.38 (m, 3H), 2.80-2.93 (m, 1H), 3.45-3.61 (m, 3H), 4.79-5.20 (m, 4H), 7.00-7.42 (m, 4H), 7.77-8.00 (m, 5H), 8.41-8.49 (m, 1H).

### Example 72

### 4-(3-Phenylcarbamoylmethyl-phenylcarbamoyl)-2-pyridin-4-yl-thiazolidine-3-carboxylic acid benzyl ester (Compound 7072)

Prepared using experimental procedure described for compound 7031 (Example 31). MS: 553.7 (M+H⁺)

### Example 73

### 4-{4-[(Biphenyl-4-sulfonylamino)-methyl]-phenylcarbamoyl}-2-pyridin-4-yl-thiazolidine-3-carboxylic acid benzyl ester (Compound 7073)

This was prepared using a procedure similar to compound 7111 (Example 111). MS: 665 (M+H⁺).

### Example 74

### 3-(Tetrahydro-furan-2-carbonyl)-2-(tetrahydro-pyran-4-yl)-thiazolidine-4-carboxylic acid [4-(4-cyclopropylcarbamoyl-phenyl)-thiazol-2-yl]-amide (Compound 7074)

Prepared using experimental procedure described for compound 7104 (Example 104). MS: 557.1 (M+H⁺); ¹H NMR (DMSO-d₆) δ (ppm) 12.73 (s, 0.3H), 12.58 (s, 0.4H), 8.45-8.44 (d, 1H), 8.0-7.79 (m, 5H), 5.32-5.29 (m, 0.7H), 5.12-5.09 (m, 0.54H), 4.87-4.4.78 (m, 1H), 4.40-4.38 (m, 0.4H), 3.87-3.78 (m, 5H), 3.70-3.45 (bs, under water), 3.44-3.11 (m, 3H), 2.87-2.81 (m 1H), 2.25-2.22 (m, 0.7H), 1.96-1.69 (m, 6H), 1.37-1.23 (m, 2H), 0.73-0.68 (m, 4H).

### Example 75

### 4-(4-Benzylcarbamoyl-phenylcarbamoyl)-2-pyridin-4-yl-thiazolidine-3-carboxylic acid benzyl ester (Compound 7075)

Synthesized using experimental procedure for compound 7031 (Example 31). MS: 553.2 (M+H⁺); H¹ NMR (DMSO-d₆): δ (ppm) 10.84 (br s, 1H), 8.89 (m, 3H), 7.90 (d, 2H), 7.70 (t, 2H), 7.23 (m, 9H), 6.49 (s, 1H), 5.00 (m, 2H), 4.88 (br s, 1H), 4.46 (s, 2H), 3.63 (m, 1H), 3.15 (m, 2H).

### Example 76

### 4-(4-Phenylcarbamoyl-phenylcarbamoyl)-2-pyridin-4-yl-thiazolidine-3-carboxylic acid benzyl ester (Compound 7076)

Synthesized using experimental procedure for compound 7031 (Example 31). MS: 539.1 (M+H⁺); H¹ NMR (DMSO-d₆): δ (ppm) 10.98 (s, 1H), 10.18 (s, 1H), 8.92 (br s, 2H), 8.48 (br s, 2H), 7.94 (s, 2H), 7.53 (m, 5H), 7.13 (m, 7H), 6.51 (s, 1H), 4.99 (m, 2H), 3.64 (m, 2H), 3.17 (m, 1H).

### Example 77

### 4-[4-(Benzoylamino-methyl)-phenylcarbamoyl]-2-pyridin-4-yl-thiazolidine-3-carboxylic acid benzyl ester (compound 7077)

Prepared using the procedure for compound 7078 (Example 78). MS: 553.2 (M+H⁺).

### Example 78

### 4-[4-(tert-Butoxycarbonylamino-methyl)-phenylcarbamoyl]-2-pyridin-4-yl-thiazolidine-3-carboxylic acid benzyl ester (Compound 7078)

2-Pyridin-4-yl-thiazolidine-3,4-dicarboxylic acid 3-benzyl ester (Example 31, 0.54 g, 1.56 mmol) and (4-amino-benzyl)-carbamic acid tert-butyl ester (0.35 g, 1.56 mmol) were dissolved in DMF. HATU (0.71 g, 1.87 mmol) was added, followed by DIPEA (0.43 mL, 2.34 mmol) and the mixture stirred at room temperature for 1 hr. The solution was added dropwise into ice-water. The desired product crashed out and was isolated by filtration, washing (water) and drying to afford 0.84 g of an off-white solid (0.84 g, 99%). MS: 549.2 (M+H⁺).

### Example 79

### 4-(1-Ethoxycarbonyl-cyclobutylcarbamoyl)-2-pyridin-4-yl-thiazolidine-3-carboxylic acid benzyl ester (compound 7079)

Prepared using experimental procedure described for compound 7031 (Example 31). MS: 470.7 (M+H⁺)

### Example 80

### 4-[4-(4-Cyclopropylcarbamoyl-phenyl)-thiazol-2-ylcarbamoyl]-2-(3-morpholin-4-ylmethyl-phenyl)-thiazolidine-3-carboxylic acid benzyl ester (Compound 7080)

### (R)-2-(3-Morpholin-4-ylmethyl-phenyl)-thiazolidine-4-carboxylic acid

To a solution of L-cysteine hydrochloride monohydrate (400 mg, 2.54 mmol) in distilled water (4 mL), potassium acetate (275 mg, 2.80 mmol) was added. Once the solids went into solution, methanol (4 mL) was added followed by 3-morpholin-4-ylmethyl-benzaldehyde (625 mg, 3.05 mmol). The reaction was stirred at ambient temperature for 1.5 hours. The solvent was removed, and no further purification steps were taken.

### (R)-2-(3-Morpholin-4-ylmethyl-phenyl)-thiazolidine-3,4-dicarboxylic acid 3-benzyl ester

To a solution of (R)-2-(3-morpholin-4-ylmethyl-phenyl)-thiazolidine-4-carboxylic acid (783 mg, 2.54 mmol) in dichloromethane (15 mL), triethylamine (530 μL, 3.80 mmol), DMAP (catalytic), and carbonic acid benzyl ester 2,5-dioxo-pyrrolidin-1-yl ester (949 mg, 3.81 mmol) were added. The reaction was stirred at ambient temperature for 2 hours. The reaction was filtered and purified by reverse phase HPLC to give a diastereomeric mixture of the desired products.

### 4-[4-(4-Cyclopropylcarbamoyl-phenyl)-thiazol-2-ylcarbamoyl]-2-(3-morpholin-4-ylmethyl-phenyl)-thiazolidine-3-carboxylic acid benzyl ester (compound 7080)

(R)-2-(3-Morpholin-4-ylmethyl-phenyl)-thiazolidine-3,4-dicarboxylic acid 3-benzyl ester was dissolved in DMF (1.5 mL). DIEA (76 μL, 0.44 mmol) was added followed by HATU (83 mg, 0.22 mmol). The reaction was stirred at ambient temperature for 15 minutes. Then 4-(2-Amino-thiazol-4-yl)-N-cyclopropyl-benzamide (57 mg, 0.22 mmol) was added, and the reaction was stirred at ambient temperature for 2 hours. Due to incompletion, the reaction was heated to 50° C. overnight. The reaction was cooled, filtered, and purified by reverse phase HPLC to give a diastereomeric mixture of the desired products. MS: 684.2 (M+H⁺); H¹ NMR (DMSO-d₆): δ (ppm) 0.56-0.74 (m, 4H), 2.80-2.90 (m, 1H), 3.05-3.40 (m, 4H), 3.50-4.05 (m, 7H), 4.28-4.42 (m, 2H), 4.95-5.10 (m, 3H), 6.32 (s, 1H), 7.00-7.30 (m 4H), 7.40-7.55 (m, 2H), 7.72-8.00 (m, 6H), 8.45-8.48 (m, 1H), 10.05-10.22 (m, 1H).

### Example 81

### 2-(3-Cyano-phenyl)-4-[4-(4-cyclopropylcarbamoyl-phenyl)-thiazol-2-ylcarbamoyl]-oxazolidine-3-carboxylic acid benzyl ester (Compound 7081)

Prepared using experimental procedure described for compound 7031 (Example 31). MS: 594 (M+H⁺); ¹H NMR (DMSO-d₆) δ (ppm) 12.74 (s, 0.6H), 8.45-8.44 (d, 1H), 8.20-8.07 (m, 2H), 7.97-7.79 (m, 5H), 7.65-7.61 (m, 2H), 7.22-7.19 (m, 4H), 6.93 (m, 1H), 6.05 (m, 1H), 5.02-4.88 (m, 3H), 4.36-4.31 (m, 2H), 2.88-2.82 (m, 1H), 1.23-1.11 (m, 3H), 0.73-0.55, (m, 3H).

### Example 82

### 4-(4-Cyclopropylcarbamoyl-phenylcarbamoyl)-2-pyridin-3-yl-thiazolidine-3-carboxylic acid benzyl ester (Compound 7082)

This was prepared by coupling 2-pyridin-3-yl-thiazolidine-3,4-dicarboxylic acid 3-benzyl ester and 4-amino-N-cyclopropyl-benzamide using an amide coupling procedure similar to compound 7078 (Example 78). MS: 503.1 (M+H⁺).

### Example 83

### 4-{4-[(Cyclopropanecarbonyl-amino)-methyl]-phenylcarbamoyl}-2-pyridin-4-yl-thiazolidine-3-carboxylic acid benzyl ester (Compound 7083)

Prepared using the procedure for compound 7078 (Example 78). MS: 517.2 (M+H⁺).

### Example 84

### 4-[4-(4-Methyl-1H-benzoimidazol-2-ylmethyl)-phenylcarbamoyl]-2-pyridin-4-yl-thiazolidine-3-carboxylic acid benzyl ester (Compound 7084)

### 4-(4-Carboxymethyl-phenylcarbamoyl)-2-pyridin-4-yl-thizolidine-3-carboxylic acid benzyl ester

2-Pyridin-4-yl-thizolidine-3,4-dicarboxylic acid 3-benzyl ester (210 mg, 0.61 mmol) was combined with PyBrOP (284.4 mg, 0.61 mmol). This mixture was dissolved in 3 ml of DMF and DIEA (0.21 mL, 1.22 mmol) was added. This solution was stirred at room temperature for 10 minutes and (110.1 mg, 0.73 mmol) of (4-amino-phenyl)-acetic acid was added. Reaction mixture was stirred at room temperature for 3 hours. The crude mixture was concentrated and purified using silica gel chromatography.

### 4-{4-[(2-Amino-5-methyl-phenylcarbamoyl)-methyl]-phenylcarbamoyl}-2-pyridin-4-yl-thizolidine-3-carboxylic acid benzyl ester

4-(4-Carboxymethyl-phenylcarbamoyl)-2-pyridin-4-yl-thizolidine-3-carboxylic acid benzyl ester, IS2342-80, (300 mg, 0.63 mmol) was combined with (263 mg, 0.66 mmol) of HATU. This mixture was dissolved in 3.5 mL of DMF and DIEA (0.22 mL, 1.86 mmol) was added. This solution was stirred at room temperature for 30 minutes at which point 4-methyl-benzene-1,2 diamine (85.8 mg, 0.63 mmol) was added. This mixture was stirred at room temperature overnight. It was concentrated and purified using reverse phase HPLC.

### 4-[4-(4-Methyl-1H-benzoimidazol-2-ylmethyl)-phenylcarbamoyl]-2-pyridin-4-yl-thizolidine-3-carboxylic acid benzyl ester (compound 7084)

4-{4-[(2-Amino-5-methyl-phenylcarbamoyl)-methyl]-phenylcarbamoyl}-2-pyridin-4-yl-thizolidine-3-carboxylic acid benzyl ester, IS2342-86, 110 mg was dissolved in dry acetic acid. Reaction mixture was heated at 70° C. for 30 minutes, and evaporated to dryness. It was purified using reverse phase HPLC. MS: 564.7 (M+H⁺); H¹ NMR δ (ppm) (DMSO-d₆): δ (ppm) 10.55 (d, 1H), 8.84 (d, 2H), 7.95 (d, 2H), 7.62-7.11 (m, 1H), 6.86 (s, 1H), 6.33 (d, 1H), 5.26 (d, 1H), 4.96 (m, 2H), 4.50 (s, 2H), 3.69 (m, 3H), 3.42 (m, 2H), 2.59 (s, 3H).

### Example 85

### 3-Cyclopropanecarbonyl-2-(tetrahydro-pyran-4-yl)-thiazolidine-4-carboxylic acid [4-(4-cyclopropylcarbamoyl-phenyl)-thiazol-2-yl]-amide (Compound 7085)

To a mixture of (R)-2-(tetrahydro-pyran-4-yl)-thiazolidine-4-carboxylic acid [4-(4-cyclopropylcarbamoyl-phenyl)-thiazol-2-yl]-amid, 0.08 g, 0.14 mmol) and TEA (0.098 mL, 0.7 mmol) in DCM (3 mL) at 0° C. was added cyclopropylacyl-chloride (0.025 mL, 0.28 mmol). The mixture was stirred at room temperature overnight. One more equivalent of acylchloride was added and stirred for an additional 8 h. Water was added. The organic phase was separated, dried and concentrated to give the crude product. Purification by reverse phase HPLC gave the desired product. MS: 527.2 (M+H⁺); ¹H NMR (DMSO-d₆): δ (ppm) 12.55 (s, 0.6), 8.45-8.44 (d, 1H), 7.93-7.77 (m, 4H), 5.41-5.38 (d, 1H), 4.91-4.85 (t, 1H), 4.13-4.08 (m, 1H), 3.95-3.88 (m 2H), 3.47-3.15 (bs, under water), 2.87-2.82 (m, 1H), 2.10-1.75 (m, 4H), 1.50-1.22 (m, 3H), 0.82-0.62 (m, 8H).

### Example 86

### 4-[4-(2-Hydroxyl-ethylcarbamoyl)-phenylcarbamoyl]-2-pyridin-4-yl-thiazolidine-3-carboxylic acid benzyl ester (Compound 7086)

Synthesized using experimental procedure for compound 7031 (Example 31). MS: 507.2 (M+H⁺).

### Example 87

### 4-[4-(4-Cyclopropylcarbamoyl-phenyl)-thiazol-2-ylcarbamoyl]-2-(4-morpholin-4-ylmethyl-phenyl)-thiazolidine-3-carboxylic acid benzyl ester (Compound 7087)

Prepared from 4-morpholin-4-ylmethyl-benzaldehyde using experimental procedures described for compound 7080 (Example 80). MS: 684.2 (M+H⁺); H¹ NMR (DMSO-d₆): δ (ppm) 0.55-0.75 (m, 4H), 2.81-2.91 (m, 1H), 3.01-3.34 (m, 5H), 3.50-3.72 (m, 3H), 3.88-4.01 (m, 2H), 4.29-4.40 (m, 2H), 4.90-5.10 (m, 3H), 6.33-6.35 (m, 1H), 6.97-7.27 (m, 4H), 7.47-7.56 (m, 2H), 7.82-8.00 (m, 6H), 8.45-8.49 (m, 1H), 10.05-10.26 (m, 1H).

### Example 88

### 2-(4-Carboxy-butyl)-4-[4-(4-cyclopropylcarbamoyl-phenyl)-thiazol-2-ylcarbamoyl]-thiazolidine-3-carboxylic acid benzyl ester (compound 7088)

Prepared from 4-[4-(4-cyclopropylcarbamoyl-phenyl)-thiazol-2-ylcarbamoyl]-2-(4-methoxycarbonyl-butyl)-thiazolidine-3-carboxylic acid benzyl ester using the experimental procedures for compound 7065 (Example 65). MS: 609.2 (M+H⁺); H¹ NMR (DMSO-d₆): δ (ppm) 0.53-0.76 (m, 4H), 1.26-1.81 (m, 4H), 1.90-2.06 (m, 1H), 2.11-2.29 (m, 3H), 2.82-2.90 (m, 1H), 3.15-3.35 (m, 2H), 4.78-5.15 (m, 4H), 7.00-7.20 (m, 2H), 7.27-7.40 (m, 2H), 7.78-7.97 (m, 5H), 8.41-8.49 (m, 1H).

### Example 89

### 4-(4-Aminomethyl-phenylcarbamoyl)-2-pyridin-4-yl-thiazolidine-3-carboxylic acid benzyl ester (Compound 7089)

4-[4-(tert-Butoxycarbonylamino-methyl)-phenylcarbamoyl]-2-pyridin-4-yl-thiazolidine-3-carboxylic acid benzyl ester (0.82 g, 1.49 mmol) was dissolved in DCM (40 mL) and HCl (4M anhydrous in 1,4-dioxane, 5 mL) was added. The mixture was stirred for 2 hrs. Ether (40 mL) was added and the solids were filtered, washed (ether) and dried to afford the desired product as an off-white solid (0.76 g, 100%). MS: 449.1 (M+H⁺).

### Example 90

### 4-[4-(4-Cyclopropylcarbamoyl-phenyl)-thiazol-2-ylcarbamoyl]-2-(tetrahydropyran-4-yl)-thiazolidine-3-carboxylic acid tert-butyl ester (Compound 7090)

### (R)-2-(Tetrahydropyran-4-yl)-thiazolidine-4-carboxylic acid

A mixture of L-cysteine hydrochloride monohydrate (7.0 g, 40.0 mmol), tetrahydropyran-4-carbaldehyde (5.0 g, 43.8 mmol), KOH (4.3 g, 43.8 mmol), MeOH (40 mL), and water (60 mL) was stirred at room temperature for 1 h. The solid was filtered and dried to give the desired product (R)-2-(tetrahydropyran-4-yl)-thiazolidine-4-carboxylic acid; MS: 218.1 (M+H⁺).

### (R)-2-(Tetrahydropyran-4-yl)-thiazolidine-3,4-dicarboxylic acid 3-tert-butyl ester

To a stirred mixture of 2-(tetrahydropyran-4-yl)-thiazolidine-4-carboxylic acid, 3.51 g, 16.2 mmol) in water (60 mL) and dioxane (40 mL) was added solid. NaHCO₃ (3.0 g, 35.7 mmol) followed by (BOC)₂O (0.67 g, 3.1 mmol) at 0° C. The mixture was stirred at room temperature for 3 h then acidified with citric acid. The separated organic phase was dried (Na₂SO₄) and concentrated to give the crude product (R)-2-(tetrahydropyran-4-yl)-thiazolidine-3,4-dicarboxylic acid 3-tert-butyl ester; MS: 318.1 (M+H⁺).

### 4-[4-(4-Cyclopropylcarbamoyl-phenyl)-thiazol-2-ylcarbamoyl]-2-(tetrahydropyran-4-yl)-thiazolidine-3-carboxylic acid tert-butyl ester (compound 7090)

A mixture of (R)-2-(tetrahydropyran-4-yl)-thiazolidine-3,4-dicarboxylic acid 3-tert-butyl ester, 4.83 g, 15.22 mmol), HATU (6.5 g, 17.0 mmol), and DIEA (4.4 mL, 35 mmol) in DMF (100 mL) was stirred at room temperature for 1 h. 4-(2-Amino-thiazol-4-yl)-N-cyclopropyl-benzamide, 4.41 g, 17.1 mmol) was added and the reaction mixture was stirred overnight. Water (200 mL) was added to the reaction mixture. The aqueous layer was separated and extracted with EtOAc (5×100 mL). The combined organic layers was washed with brine, dried (Na₂SO₄), and concentrated to give the crude product. Purification by ISCO furnished the desired product. MS: 559 (M+H⁺); ¹H NMR (DMSO-d₆): δ (ppm) 12.55 (s, 0.8H), 8.39-8.38 (d, 1H), 7.91-7.74 (m, 5H), 4.84-4.64 (m, 2H), 3.85-3.82 (m, 2H), 3.39-3.33 (m, 2H), 3.27-3.06 (m, 5H), 2.83-2.76 (m, 2H), 1.81-1.65 (m, 3H), 1.36-1.15 (m, 9H), 0.77-0.52 (m, 4H).

### Example 91

### 4-[4-(4-Cyclopropylcarbamoyl-phenyl)-thiazol-2-ylcarbamoyl]-2-(2-methylcarbamoyl-ethyl)-thiazolidine-3-carboxylic acid benzyl ester (Compound 7091)

Prepared from 2-(2-carboxy-ethyl)-4-[4-(4-cyclopropylcarbamoyl-phenyl)-thiazol-2-ylcarbamoyl]-thiazolidine-3-carboxylic acid benzyl ester using the experimental procedures for compound 7125 (Example 125). MS: 594.1 (M+H⁺); H¹ NMR (DMSO-d₆): δ (ppm) 0.52-0.74 (m, 4H), 1.88-2.28 (m, 4H), 2.50-2.59 (m, 3H), 2.79-2.90 (m, 1H), 3.40-3.60 (m, 2H), 4.76-5.25 (m, 4H), 7.00-7.40 (m, 4H), 7.70-7.98 (m, 6H), 8.41-8.48 (m, 1H).

### Example 92

### 3-(1-Methyl-1H-imidazole-2-carbonyl)-2-(tetrahydro-pyran-4-yl)-thiazolidine-4-carboxylic acid [4-(4-cyclopropylcarbamoyl-phenyl)-thiazol-2-yl]-amide (Compound 7092)

Prepared using experimental procedure described for compound 7104 (Example 104). MS: 567.1 (M+H⁺); ¹H NMR (DMSO-d₆): δ (ppm) 13.04 (bs, 0.3H), 12.71 (bs, 0.3H), 8.46-8.44 (d, 1H), 7.94-7.77 (m, 4H), 7.06-6.97 (m, 1H), 6.61-6.59 (m, 0.4H), 5.56 (m, 0.3H), 5.53-5.01 (m, 0.3H), 4.31 (m, 0.3H), 4.01 (bs, underwater peak), 3.83-3.52 (m, 4H), 3.39-3.22 (m, 1H), 3.22-2.18 (m, 3H), 2.88-2.82 (m, 1H), 1.92-1.69 (m, 3H), 1.34-1.14 (m, 2H), 0.73-0.55 (m, 4H).

### Example 93

### 3-[4-(4-Cyclopropylcarbamoyl-phenyl)-thiazol-2-ylcarbamoyl]-1-thia-4-aza-spiro[4.5]decane-4-carboxylic acid benzyl ester (Compound 7093)

Prepared from cyclohexanone using experimental procedures described for compound 7114 (Example 114). MS: 577.2 (M+H⁺); H¹ NMR (DMSO-d₆): δ (ppm) 0.54-0.75 (m, 4H), 1.00-1.85 (m, 10H), 2.01-2.13 (m, 1H), 2.55-2.90 (M, 4H), 3.06-3.18 (m, 1H), 4.88-5.18 (m, 3H) 7.06-7.46 (m, 5H), 7.78-8.00 (m, 4H), 8.42-8.49 (m, 1H).

### Example 94

### 3-(4-Phenyl-thiazol-2-ylcarbamoyl)-8-oxa-1-thia-4-aza-spiro[4.5]decane-4-carboxylic acid benzyl ester (Compound 7094)

Prepared from tetrahydro-pyran-4-one and 4-phenyl-thiazol-2-ylamine using experimental procedures described for compound 7114 (Example 114). MS: 496.1 (M+H⁺); H¹ NMR (DMSO-d₆): δ (ppm) 1.93-2.06 (m, 1H), 3.13-3.47 (m, 2H), 3.50-3.68 (m, 2H), 3.75-3.89 (m, 2H), 4.21-4.38 (m, 2H), 5.25-5.57 (m, 4H), 7.40-7.56 (m, 4H), 7.70-7.80 (m, 4H), 7.99-8.03 (m, 1H), 8.19-8.26 (m, 2H).

### Example 95

### 4-[4-(4-Cyclopropylcarbamoyl-phenyl)-thiazol-2-ylcarbamoyl]-2-(tetrahydropyran-4-ylmethyl)-oxazolidine-3-carboxylic acid benzyl ester (Compound 7095)

Prepared using experimental procedure described for compound 7031 (Example 31). MS: 591 (M+H⁺); ¹H NMR (DMSO-d₆) δ (ppm) 12.57 (s, 1H), 8.45-8.44 (d, 2H), 7.95-7.81 (m, 5H), 7.36-7.17 (m, 4H), 5.26-5.07 (m, 3H), 4.71 (bs, 1H), 4.26-3.77 (m, under water), 3.24-3.15 (m, 2H), 2.87-2.79 (m, 1H), 1.87-1.63 (m, 5H), 1.21-1.14 (m, 2H), 0.72-0.54, (m, 4H).

### Example 96

### 4-[4-(4-Cyclopropylcarbamoyl-phenyl)-thiazol-2-ylcarbamoyl]-2-methyl-2-pyridin-4-yl-thiazolidine-3-carboxylic acid benzyl ester (Compound 7096)

Prepared from 1-pyridin-4-yl-ethanone using the experimental procedures for compound 7109 (Example 109). The product was converted to the HCl salt. After dissolving the product in a minimum amount of acetonitrile and cooling the solution in dry ice, 2.0M HCl in diethyl ether was added until precipitate crashed out of solution. The mixture was centrifuged, and the liquid was decanted. Additional cold diethyl ether was added, and the mixture was again centrifuged and the liquid decanted. The resulting solid was dried to give the HCl salt of the desired product. MS: 623.2 (M+H⁺); H¹ NMR (DMSO-d₆): δ (ppm) 0.51-0.76 (m, 4H), 2.27-2.42 (m, 3H), 2.80-2.90 (m, 1H), 3.35-3.55 (m, 2H), 4.84-5.15 (m, 2H), 5.47-5.56 (m, 1H), 6.82-6.87 (m, 1H), 7.12-7.26 (m, 4H), 7.83-8.09 (m, 7H), 8.45-8.51 (m, 1H), 8.68-8.85 (m, 2H).

### Example 97

### 4-[4-(Phenylacetylamino-methyl)-phenylcarbamoyl]-2-pyridin-4-yl-thiazolidine-3-carboxylic acid benzyl ester (Compound 7097)

Prepared using the procedure for compound 7078 (Example 78). MS: 567.2 (M+H⁺).

### Example 98

### 3-{3-Benzyloxycarbonyl-4-[4-(4-cyclopropylcarbamoyl-phenyl)-thiazol-2-ylcarbamoyl]-thiazolidin-2-yl}-piperidine-1-carboxylic acid tert-butyl ester (Compound 7098)

### 3-((R)-4-Carboxy-thiazolidin-2-yl)-piperidine-1-carboxylic acid tert-butyl ester

Prepared from 3-formyl-piperidine-1-carboxylic acid tert-butyl ester using the experimental procedures in Example 80.

### (R)-2-(1-tert-Butoxycarbonyl-piperidin-3-yl)-thiazolidine-3,4-dicarboxylic acid 3-benzyl ester

To a solution of phenyl-methanol (360 μL, 3.48 mmol) in acetonitrile (6 mL), triethylamine (970 μL, 6.96 mmol) was added followed by carbonic acid bis-(2,5-dioxo-pyrrolidin-1-yl) ester (890 mg, 3.47 mmol). The reaction was stirred at ambient temperature for 45 minutes. The solvent was removed, and the residue was redissolved in dry dichloromethane (6 mL). Then triethylamine (485 μL, 3.47 mmol), DMAP (catalytic), and 3-((R)-4-carboxy-thiazolidin-2-yl)-piperidine-1-carboxylic acid tert-butyl ester (735 mg, 2.32 mmol) were added. The reaction was stirred at ambient temperature for 5 hours. The reaction was filtered and purified by reverse phase HPLC to give a diastereomeric mixture of the desired products.

### 3-{3-Benzyloxycarbonyl-4-[4-(4-cyclopropylcarbamoyl-phenyl)-thiazol-2-ylcarbamoyl]-thiazolidin-2-yl}-piperidine-1-carboxylic acid tert-butyl ester (compound 7098)

Prepared from (R)-2-(1-tert-butoxycarbonyl-piperidin-3-yl)-thiazolidine-3,4-dicarboxylic acid 3-benzyl ester using the experimental procedures for compound 7080 (Example 80). MS: 692.2 (M+H⁺); H¹ NMR (DMSO-d₆): δ (ppm) 0.53-0.76 (m, 4H), 1.30-1.45 (m, 12H), 2.80-2.90 (m, 2H), 3.09-3.25 (m, 2H), 3.45-3.55 (m, 4H), 4.79-5.16 (m, 5H), 7.00-7.39 (m, 5H), 7.77-7.98 (m, 5H), 8.43-8.48 (m, 1H).

### Example 99

### 4-[4-(4-Cyclopropylcarbamoyl-phenyl)-thiazol-2-ylcarbamoyl]-2-(4-methylcarbamoyl-butyl)-thiazolidine-3-carboxylic acid benzyl ester (Compound 7099)

Prepared from 2-(4-carboxy-butyl)-4-[4-(4-cyclopropylcarbamoyl-phenyl)-thiazol-2-ylcarbamoyl]-thiazolidine-3-carboxylic acid benzyl ester using the experimental procedures for compound 7125 (Example 125). MS: 622.2 (M+H⁺); H¹ NMR (DMSO-d₆): δ (ppm) 0.53-0.76 (m, 4H), 1.10-1.80 (m, 7H), 1.90-2.11 (m, 3H), 2.51-2.58 (m, 3H), 2.80-2.90 (m, 1H), 4.77-5.15 (m, 4H), 7.01-7.40 (m, 4H), 7.62-7.99 (m, 5H), 8.42-8.48 (m, 1H).

### Example 100

### 4-(3-Benzylcarbamoyl-phenylcarbamoyl)-2-pyridin-4-yl-thiazolidine-3-carboxylic acid benzyl ester (Compound 7100)

Synthesized using experimental procedure for compound 7031 (Example 31). MS: 553.1 (M+H⁺); H¹ NMR (DMSO-d₆): δ (ppm) 10.58 (s, 1H), 8.90 (br s, 1H), 8.49 (t, 2H), 8.09 (d, 2H), 7.62 (d, 2H), 7.22 (m, 10H), 6.35 (d, 1H), 5.26 (m, 1H), 4.96 (m, 2H), 4.44 (s, 2H), 3.64 (m, 2H), 3.13 (m, 2H).

### Example 101

### 2-Benzyl-4-[4-(4-cyclopropylcarbamoyl-phenyl)-thiazol-2-ylcarbamoyl]-thiazolidine-3-carboxylic acid benzyl ester (Compound 7101)

Prepared from phenyl-acetaldehyde using experimental procedures described for compound 7080 (Example 80). MS: 599.1 (M+H⁺); H¹ NMR (DMSO-d₆): δ (ppm) 0.54-0.75 (m, 4H), 2.81-3.04 (m, 2H), 3.35-3.57 (m, 3H), 4.86-5.30 (m, 4H), 7.04-7.40 (m, 10H), 7.82-8.00 (m, 4H), 8.43-8.48 (m, 1H).

### Example 102

### 4-[4-(4-Cyclopropylcarbamoyl-phenyl)-thiazol-2-ylcarbamoyl]-2-(5-oxo-5-piperidin-1-yl-pentyl)-thiazolidine-3-carboxylic acid benzyl ester (Compound 7102)

Prepared from 2-(4-carboxy-butyl)-4-[4-(4-cyclopropylcarbamoyl-phenyl)-thiazol-2-ylcarbamoyl]-thiazolidine-3-carboxylic acid benzyl ester and piperidine using experimental procedures described for compound 7125 (Example 125). MS: 676.2 (M+H⁺); H¹ NMR (DMSO-d₆): δ (ppm) 0.52-0.75 (m, 4H), 1.26-1.62 (m, 9H), 1.26-1.80 (m, 2H), 1.90-2.10 (m, 2H), 2.16-2.35 (m, 3H), 2.79-2.90 (m, 2H), 3.45-3.54 (m, 3H), 4.77-5.20 (m, 4H), 7.02-7.45 (m, 5H), 7.80-8.03 (m, 4H), 8.41-8.50 (m, 1H).

### Example 103

### 3-(2-Methyl-pentanoyl)-2-(tetrahydro-pyran-4-yl)-thiazolidine-4-carboxylic acid [4-(4-cyclopropylcarbamoyl-phenyl)-thiazol-2-yl]-amide (Compound 7103)

Prepared using experimental procedure described for compound 7104 (Example 104). MS: 557.1 (M+H⁺); ¹H NMR (DMSO-d₆) δ (ppm) 12.84 (s, 0.2H), 12.56 (s, 0.4H), 8.45 (d, 1H), 7.97-7.78 (m, 4H), 5.33 (m, 0.3H), 5.1-4.84 (m, 2H), 3.92 (m, 2H), 3.64 (m, 0.44H), 3.48-3.41 (m, 11H), 3.33-3.12 (bs, under water), 2.84 (m, 2H), 2.49 (m, 9H), 1.88-1.85 (m, 3H), 1.35-1.22 (m, 6H), 1.02-0.57 (m, 10H).

### Example 104

### 3-(2-Cyclopropyl-acetyl)-2-(tetrahydropyran-4-yl)-thiazolidine-4-carboxylic acid [4-(4-cyclopropylcarbamoyl-phenyl)-thiazol-2-yl]-amide (Compound 7104)

### (R)-2-(Tetrahydro-pyran-4-yl)-thiazolidine-4-carboxylic acid [4-(4-cyclopropyl-carbamoylphenyl)-thiazol-2-yl]-amid

A mixture of compound 7090 (2.5 g, 4.5 mmol) and TFA (10 mL) in DCM (20 mL) was stirred at room temperature for 1 h, and then concentrated to give the desired product (R)-2-(tetrahydropyran-4-yl)-thiazolidine-4-carboxylic acid [4-(4-cyclopropylcarbamoyl-phenyl)-thiazol-2-yl]-amid; MS: 459.1 (M+H⁺).

### 3-(2-Cyclopropyl-acetyl)-2-(tetrahydropyran-4-yl)-thiazolidine-4-carboxylic acid [4-(4-cyclopropylcarbamoyl-phenyl)-thiazol-2-yl]-amide (Compound 7104)

A mixture of cyclopropyl-acetic acid (0.026 mL, 0.3 mmol), HATU (0.11 g, 0.3 mmol), and TEA (0.11 mL) in DMF (2.0 mL) was stirred at room temperature for 1 h. (R)-2-(Tetrahydropyran-4-yl)-thiazolidine-4-carboxylic acid [4-(4-cyclopropylcarbamoyl-phenyl)-thiazol-2-yl]-amid, 0.08 g, 0.14 mmol) was added and the mixture was stirred at room temperature overnight. It was then heated to 50° C. overnight. Purification by reverse phase HPLC furnished the desired product. MS: 541.2 (M+H⁺); ¹H NMR (DMSO-d₆) δ (ppm) 12.68 (s, 0.2H), 12.50 (s, 6H), 8.39-8.38 (d, 1H), 7.90-7.21 (m, 5H), 5.26-5.23 (m, 0.2H), 5.00-4.83 (m, 2H), 3.83-382 (m, 2H), 3.53-3.04 (m, under water peak), 2.81-2.76 (m, 1H), 1.87-1.72 (m, 4H), 1.27-1.17 (m, 3H), 0.90-0.87 (m, 1H), 0.64-0.36 (m, 7H), 0.8-0076 (m, 2H).

### Example 105

### 4-[4-(Benzylcarbamoyl-methyl)-phenylcarbamoyl]-2-pyridin-4-yl-thiazolidine-3-carboxylic acid benzyl ester (Compound 7105)

Prepared using experimental procedure described for compound 7031 (Example 31). MS: 567.7 (M+H⁺)

### Example 106

### 4-{[4-(4-Cyclopropylcarbamoyl-phenyl)-thiazol-2-yl]-methyl-carbamoyl}-2-pyridin-4-yl-thiazolidine-3-carboxylic acid benzyl ester (Compound 7106)

4-(2-Bromo-acetyl)-benzoic acid and methyl-thiourea were used to synthesize 4-(2-methylamino-thiazol-4-yl)-benzoic acid. N-Cyclopropyl-4-(2-methylamino-thiazol-4-yl)-benzamide was synthesized through coupling with cyclopropyl amine via amide coupling. This was used in a final amide coupling with 2-pyridin-4-yl-thiazolidine-3,4-dicarboxylic acid 3-benzyl ester to afford the desired compound. MS: 600.2 (M+H⁺); ¹H NMR (DMSO-d₆): 1(Ppm) 8.91 (m, 2H), 8.45 (m, 2H), 7.96 (m, 6H), 7.11 (m, 5H), 6.52 (m, 1H), 5.55 (t, 1H), 4.99 (m, 3H), 3.90 (m under water peak), 3.22 (br t, 1H), 2.85 (m, 2H), 0.62 (m, 4H).

### Example 107

### 4-(4-Cyclopropylcarbamoyl-phenylcarbamoyl)-2-(tetrahydro-pyran-4-yl)-thiazolidine-3-carboxylic acid benzyl ester (Compound 7107)

Synthesized using experimental procedure for compound 7025 (Example 25). MS: 510.71 (M+H⁺).

### Example 108

### 4-[4-(4-Cyclopropylcarbamoyl-phenyl)-thiazol-2-ylcarbamoyl]-2-(4-methoxycarbonyl-butyl)-thiazolidine-3-carboxylic acid benzyl ester (Compound 7108)

Prepared from 6-oxo-hexanoic acid methyl ester using the experimental procedures for compound 7080 (Example 80). MS: 623.2 (M+H⁺); H¹ NMR (DMSO-d₆): δ (ppm) 0.54-0.75 (m, 4H), 1.30-1.80 (m, 2H), 2.80-2.92 (m, 1H), 3.14-3.62 (m, 1H), 4.88-5.18 (m, 4H), 7.01-7.40 (m, 5H), 7.80-7.99 (m, 4H), 8.41-8.48 (m, 1H).

### Example 109

### 2-(4-Acetyl-phenyl)-4-[4-(4-cyclopropylcarbamoyl-phenyl)-thiazol-2-ylcarbamoyl]-thiazolidine-3-carboxylic acid benzyl ester (Compound 7109)

### (R)-2-(4-Acetyl-phenyl)-thiazolidine-4-carboxylic acid

Prepared from 4-acetyl-benzaldehyde using experimental procedures described in Example 80.

### (R)-2-(4-Acetyl-phenyl)-thiazolidine-3,4-dicarboxylic acid 3-benzyl ester

(R)-2-(4-Acetyl-phenyl)-thiazolidine-4-carboxylic acid (335 mg, 1.33 mmol) was dissolved in DMF (8 mL). The solution was cooled to 0° C., and DIEA (464.5 μL, 2.67 mmol) was added followed by benzyl chloroformate (285.5 μL, 2.00 mmol). The reaction was stirred at 0° C. and allowed to warm to ambient temperature over 3 hours. The reaction was filtered and purified by reverse phase HPLC to give a diastereomeric mixture of the desired products.

### 2-(4-Acetyl-phenyl)-4-[4-(4-cyclopropylcarbamoyl-phenyl)-thiazol-2-ylcarbamoyl]-thiazolidine-3-carboxylic acid benzyl ester (compound 7109)

Prepared from (R)-2-(4-acetyl-phenyl)-thiazolidine-3,4-dicarboxylic acid 3-benzyl ester using experimental procedures described for compound 7080 (Example 80). MS: 627.2 (M+H⁺); H¹ NMR (DMSO-d₆): δ (ppm) 0.56-0.76 (m, 4H), 2.59 (s, 3H), 2.81-2.92 (m, 1H), 3.12-3.24 (m, 1H), 3.50-3.59 (m, 1H), 4.90-5.11 (m, 3H), 6.33-6.39 (m, 1H), 6.95-7.24 (m, 4H), 7.82-8.00 (m, 8H), 8.43-8.49 (m, 1H).

### Example 110

### 4-(1-Methoxycarbonyl-cyclopropylcarbamoyl)-2-pyridin-4-yl-thiazolidine-3-carboxylic acid benzyl ester (Compound 7110)

Prepared using experimental procedure described for compound 7031 (Example 31). MS: 442.7 (M+H⁺).

### Example 111

### 4-[4-(Benzenesulfonylamino-methyl)-phenylcarbamoyl]-2-pyridin-4-yl-thiazolidine-3-carboxylic acid benzyl ester (Compound 7111)

4-(4-Aminomethyl-phenylcarbamoyl)-2-pyridin-4-yl-thiazolidine-3-carboxylic acid benzyl ester (0.19 g, 0.48 mmol) was dissolved in DCM (2.5 mL) and benzenesulfonyl chloride (0.062 mL, 0.48 mmol) was added followed by dropwise addition of DIPEA (0.26 mL, 1.44 mmol). The mixture was stirred for 2 hrs followed by partitioning between ethyl acetate and water. The organic layer was separated, washed (water) and dried to afford the crude product. This was redissolved in 0.1 N HCl and acetonitrile and lyophilized to afford the desired product as a solid (0.0491 g, 17%). MS: 589.5 (M+H⁺); ¹H NMR (DMSO-d₆): δ (ppm) 10.29 (m, 1H), 8.80 (d, 1H), 8.72 (d, 1H), 8.11 (t, 1H), 8.01 (d, 1H), 7.92 (d, 1H), 7.79 (d, 2H), 7.56 (m, 5H), 7.18 (m, 6H), 6.85 (d, 1H), 6.33 (m, 1H), 5.10 (m, 3H), 3.92, 3.67, 3.39 (br m under water peak).

### Example 112

### 4-(3-Phenylcarbamoyl-phenylcarbamoyl)-2-pyridin-4-yl-thizolidine-3-carboxylic acid benzyl ester (Compound 7112)

Synthesized using experimental procedure for compound 7031 (Example 31). MS: 539.1 (M+H⁺); H¹ NMR (DMSO-d₆): δ (ppm) 10.87 (s, 1H), 10.31 (s, 1H), 8.91 (br s, 2H), 8.51 (d, 2H), 8.31 (m, 2H), 7.75 (m, 5H), 7.33-7.07 (m, 5H), 6.51 (d, 1H), 4.99 (m, 3H), 3.64 (m, 2H), 3.42 (m, 1H), 3.26 (m, 1H).

### Example 113

### 4-(4-Phenethylcarbamoyl-phenylcarbamoyl)-2-pyridin-4-yl-thiazolidine-3-carboxylic acid benzyl ester (Compound 7113)

Synthesized using experimental procedure for compound 7031 (Example 31). MS: 567.7 (M+H⁺); H¹ NMR (DMSO-d₆): δ (ppm) 10.73 (s, 1H), 8.84 (br s, 2H), 8.51 (t, 2H), 8.36 (d, 2H), 7.81 (d, 2H), 7.70 (m, 2H), 7.23 (m, 8H), 6.45 (s, 1H), 5.09 (m, 2H), 4.92 (br s, 1H), 3.82-3.32 (m, 3H), 2.95 (m, 4H).

### Example 114

### 3-[4-(4-Cyclopropylcarbamoyl-phenyl)-thiazol-2-ylcarbamoyl]-8-methyl-1-thia-4,8-diaza-spiro[4.5]decane-4-carboxylic acid benzyl ester (Compound 7114)

### (R)-8-Methyl-1-thia-4,8-diaza-spiro[4.5]decane-3-carboxylic acid

To a solution of L-cysteine (560 mg, 4.62 mmol) in distilled water (4 mL) and ethanol (4 mL), 1-methyl-piperidin-4-one (535 μL, 4.60 mmol) was added. The reaction was heated to 95° C. overnight. The solvent was removed, and no further purification steps were taken.

### (R)-8-Methyl-1-thia-4,8-diaza-spiro[4.5]decane-3,4-dicarboxylic acid 4-benzyl ester

Prepared from (R)-8-methyl-1-thia-4,8-diaza-spiro[4.5]decane-3-carboxylic acid using experimental procedures described in Example 109.

### 3-[4-(4-Cyclopropylcarbamoyl-phenyl)-thiazol-2-ylcarbamoyl]-8-methyl-1-thia-4,8-diaza-spiro[4.5]decane-4-carboxylic acid benzyl ester (compound 7114)

Prepared from (R)-8-methyl-1-thia-4,8-diaza-spiro[4.5]decane-3,4-dicarboxylic acid 4-benzyl ester using experimental procedures described for compound 7080 (Example 80). MS: 592.2 (M+H⁺); H¹ NMR (DMSO-d₆): δ (ppm) 0.53-0.75 (m, 4H), 1.87-2.06 (m, 1H), 2.15-2.29 (m, 1H), 2.71-3.55 (m, 12H), 4.95-5.27 (m, 3H), 7.08-7.44 (m, 4H), 7.78-7.99 (m, 5H), 8.43-8.50 (M, 1H).

### Example 115

### 4-{3-[(4-Chloro-benzylcarbamoyl)-methyl]-phenylcarbamoyl}-2-pyridin-4-yl-thiazolidine-3-carboxylic acid benzyl ester (Compound 7115)

Prepared using experimental procedure described for compound 7031 (Example 31). MS: 602.7 (M+H⁺).

### Example 116

### 4-[3-(Benzylcarbamoyl-methyl)-phenylcarbamoyl]-2-pyridin-4-yl-thiazolidine-3-carboxylic acid benzyl ester (compound 7116)

Prepared using experimental procedure described for compound 7031 (Example 31). MS: 567.7 (M+H⁺)

### Example 117

### 4-[4-(5-Methyl-1H-benzoimidazol-2-ylmethyl)-phenylcarbamoyl]-2-pyridin-4-yl-thiazolidine-3-carboxylic acid benzyl ester (Compound 7117)

Synthesized using experimental procedure for compound 7084 (Example 84). MS: 564.7 (M+H⁺); H¹ NMR (DMSO-d₆): δ (ppm) 10.51 (d, 1H), 8.77 (m, 4H), 8.27 (d, 2H), 7.92-6.84 (m, 11H), 6.42 (d, 1H), 6.28 (d, 2H), 5.22 (d, 1H), 4.96 (m, 2H), 4.47 (s, 2H), 3.63 (m, 1H), 3.15 (m, 2H).

### Example 118

### 4-[4-(4-Cyclopropylcarbamoyl-phenyl)-thiazol-2-ylcarbamoyl]-2-(2-dimethylcarbamoyl-ethyl)-thiazolidine-3-carboxylic acid benzyl ester (Compound 7118)

Prepared from 2-(2-carboxy-ethyl)-4-[4-(4-cyclopropylcarbamoyl-phenyl)-thiazol-2-ylcarbamoyl]-thiazolidine-3-carboxylic acid benzyl ester using the experimental procedures for compound 7125 (Example 125). MS: 608.2 (M+H⁺); H¹ NMR (DMSO-d₆): δ (ppm) 0.53-0.76 (m, 4H), 1.90-2.25 (m, 3H), 2.33-2.47 (m, 2H), 2.70-2.99 (m, 6H), 3.17-3.32 (m, 1H), 4.80-5.32 (m, 5H), 7.04-7.45 (m, 5H), 7.80-8.00 (m, 4H), 8.43-8.50 (m, 1H).

### Example 119

### 4-[4-(Phenethylcarbamoyl-methyl)-phenylcarbamoyl]-2-pyridin-4-yl-thiazolidine-3-carboxylic acid benzyl ester (Compound 7119)

Prepared using experimental procedure described for compound 7031 (Example 31). MS: 581.7 (M+H⁺)

### Example 120

### 2-(3-Acetyl-phenyl)-4-[4-(4-cyclopropylcarbamoyl-phenyl)-thiazol-2-ylcarbamoyl]-thiazolidine-3-carboxylic acid benzyl ester (Compound 7120)

Prepared from 3-acetyl-benzaldehyde using the experimental procedures for compound 7109 (Example 109). MS: 627.2 (M+H⁺); H¹ NMR (DMSO-d₆): δ (ppm) 0.54-0.75 (m, 4H), 2.54-2.67 (m, 3H), 2.81-2.91 (m, 1H), 3.17-3.28 (m, 1H), 3.48-3.60 (m, 1H), 4.92-5.12 (m, 3H), 6.38 (s, 1H), 6.93-7.25 (m, 4H), 7.49-7.56 (m, 1H), 7.81-8.09 (m, 7H), 8.38-8.49 (m, 2H).

### Example 121

### 4-[4-(4-Cyclopropylcarbamoyl-phenyl)-thiazol-2-ylcarbamoyl]-2-(2-methoxycarbonyl-ethyl)-thiazolidine-3-carboxylic acid benzyl ester (Compound 7121)

Prepared from 4-oxo-butyric acid methyl ester using the experimental procedures for compound 7080 (Example 80). MS: 595.2 (M+H⁺); H¹ NMR (DMSO-d₆): δ (ppm) 0.54-0.75 (m, 4H), 1.97-2.39 (m, 2H), 2.80-2.90 (m, 1H), 3.16-3.64 (m, 7H), 4.80-5.30 (m, 4H), 7.01-7.20 (m, 2H), 7.30-7.43 (m, 2H), 7.80-8.01 (m, 5H), 8.43-8.48 (m, 1H).

### Example 122

### 4-[4-(4-Cyclopropylcarbamoyl-phenyl)-thiazol-2-ylcarbamoyl]-2-(tetrahydro-furan-3-yl)-thiazolidine-3-carboxylic acid benzyl ester (Compound 7122)

Prepared from tetrahydro-furan-3-carbaldehyde using experimental procedures described for compound 7109 (Example 109). MS: 579.2 (M+H⁺); H¹ NMR (DMSO-d₆): δ (ppm) 0.54-0.75 (m, 4H), 1.60-2.05 (m, 3H), 2.68-2.90 (m, 3H), 3.22-3.80 (m, 3H), 4.80-5.25 (m, 4H), 7.08-7.21 (m, 2H), 7.28-7.42 (m, 3H), 7.81-7.98 (m, 4H), 8.43-8.48 (m, 1H).

### Example 123

### 4-(4-Carboxymethyl-phenylcarbamoyl)-2-pyridin-4-yl-thiazolidine-3-carboxylic acid benzyl ester (compound 7123)

Prepared using experimental procedure described for compound 7031 (Example 31). MS: 478.7 (M+H⁺)

### Example 124

### 4-[4-(3-Benzyl-ureidomethyl)-phenylcarbamoyl]-2-pyridin-4-yl-thiazolidine-3-carboxylic acid benzyl ester (Compound 7124)

4-(4-Aminomethyl-phenylcarbamoyl)-2-pyridin-4-yl-thiazolidine-3-carboxylic acid benzyl ester (0.11 g, 0.22 mmol), isocyanatomethyl-benzene (0.027 mL, 0.22 mmol), DIPEA (0.08 mL, 0.44 mmol) were stirred together in DMF (2 mL) at room temperature for 14 h. The reaction mixture was added dropwise into ice-water and solids crashed out. The solids were filtered, washed (water) and dried to afford the desired product as a white solid (0.071 g, 55%). MS: 582.3 (M+H⁺).

### Example 125

### 4-[4-(4-Cyclopropylcarbamoyl-phenyl)-thiazol-2-ylcarbamoyl]-2-(4-dimethylcarbamoyl-butyl)-thiazolidine-3-carboxylic acid benzyl ester (compound 7125)

To a solution of 2-(4-carboxy-butyl)-4-[4-(4-cyclopropylcarbamoyl-phenyl)-thiazol-2-ylcarbamoyl]-thiazolidine-3-carboxylic acid benzyl ester (57 mg, 0.09 mmol) in DMF (500 μL), DIEA (33 μL, 0.19 mmol) was added followed by HATU (36 mg, 0.09 mmol). The reaction was stirred at ambient temperature for 15 minutes. Then dimethyl-amine (47 μL, 0.09 mmol) was added, and the reaction was stirred at ambient temperature for 3 hours. Due to incompletion, the reaction was heated to 50° C. overnight. The reaction was cooled, filtered, and purified by reverse phase HPLC to give a diastereomeric mixture of the desired products. MS: 636.2 (M+H⁺); H¹ NMR (DMSO-d₆): δ (ppm) 0.54-0.75 (m, 4H), 1.21-1.83 (m, 6H), 1.90-2.08 (m, 1H), 2.18-2.34 (m, 2H), 2.75-2.98 (m, 7H), 3.15-3.31 (m, 1H), 4.78-5.18 (m, 4H), 7.02-7.40 (m, 4H), 7.80-7.99 (m, 5H), 8.43-8.50 (m, 1H).

### Example 126

### 4-(4-Phenylcarbamoylmethyl-phenylcarbamoyl)-2-pyridin-4-yl-thiazolidine-3-carboxylic acid benzyl ester (Compound 7126)

Prepared using experimental procedure described for compound 7031 (Example 31). MS: 553.7 (M+H⁺)

### Example 127

### 4-(3-carbamoyl-phenylcarbamoyl)-2-pyridin-4-yl-thiazolidine-3-carboxylic acid benzyl ester (Compound 7127)

Synthesized using experimental procedure for compound 7031 (Example 31). MS: 463.1 (M+H⁺); H¹ NMR (DMSO-d₆): δ (ppm) 10.52 (d, 1H), 8.74 (d, 1H), 8.50 (d, 1H), 8.07 (m, 2H), 7.76 (d, 2H), 7.55 (d, 2H), 7.36 (d, 2H), 7.16 (m, 4H), 6.36 (d, 1H), 5.25 (d, 1H), 4.97 (m, 2H), 3.63 (m, 1H), 3.15 (m, 2H).

### Example 128

### 4-{3-[(4-Methoxy-benzylcarbamoyl)-methyl]-phenylcarbamoyl}-2-pyridin-4-yl-thiazolidine-3-carboxylic acid benzyl ester (Compound 7128)

Prepared using experimental procedure described for compound 7031 (Example 31). MS: 597.7 (M+H⁺)

### Example 129

### 4-(1-Carboxy-cyclobutylcarbamoyl)-2-pyridin-4-yl-thiazolidine-3-carboxylic acid benzyl ester (compound 7129)

Prepared using experimental procedure described for compound 7031 (Example 31). MS: 442.7 (M+H⁺).

### Example 130

### 4-(3-Carboxymethyl-phenylcarbamoyl)-2-pyridin-4-yl-thiazolidine-3-carboxylic acid benzyl ester (Compound 7130)

Prepared using experimental procedure described for compound 7031 (Example 31). MS: 478.7 (M+H⁺)

### Example 131

### 4-[4-(4-Cyclopropylcarbamoyl-phenyl)-thiazol-2-ylcarbamoyl]-2-(2-dimethylamino-pyrimidin-5-yl)-thiazolidine-3-carboxylic acid benzyl ester (Compound 7131)

Prepared from 2-dimethylamino-pyrimidine-5-carbaldehyde using the experimental procedures for compound 7109 (Example 109). MS: 630.2 (M+H⁺); H¹ NMR (DMSO-d₆): δ (ppm) 0.52-0.76 (m, 4H), 1.21-1.29 (m, 3H), 3.05-3.17 (m, 6H), 4.89-5.11 (m, 3H) 6.12-6.18 (m, 1H), 7.03-7.40 (m, 5H), 7.79-7.99 (m, 4H), 8.42-8.48 (m, 1H), 8.60-8.68 (m, 1H).

### Example 132

### 4-[4-(2-Morpholin-4-yl-ethylcarbamoyl)-phenylcarbamoyl]-2-pyridin-4-yl-thizolidine-3-carboxylic acid benzyl ester (Compound 7132)

Synthesized using experimental procedure for compound 7031 (Example 31). MS: 576.7 (M+H⁺).

### Example 133

### 2-(1-Acetyl-piperidin-3-yl)-4-[4-(4-cyclopropylcarbamoyl-phenyl)-thiazol-2-ylcarbamoyl]-thiazolidine-3-carboxylic acid benzyl ester (Compound 7133)

### (R)-2-Piperidin-3-yl-thiazolidine-3,4-dicarboxylic acid 3-benzyl ester

A solution of (R)-2-(1-tert-butoxycarbonyl-piperidin-3-yl)-thiazolidine-3,4-dicarboxylic acid 3-benzyl ester in dichloromethane and trifluoroacetic acid (1:1) was stirred at ambient temperature overnight. Ethyl acetate was added, and the mixture was extracted with distilled water. The organic layers were combined, dried with anhydrous magnesium sulfate, filtered, and concentrated. The crude was purified by reverse phase HPLC to give a diastereomeric mixture of the desired products.

### (R)-2-(1-Acetyl-piperidin-3-yl)-thiazolidine-3,4-dicarboxylic acid 3-benzyl ester

To a solution of (R)-2-piperidin-3-yl-thiazolidine-3,4-dicarboxylic acid 3-benzyl ester (407.5 mg, 1.16 mmol) in dichloromethane (7 mL), DIEA (405 μL, 2.33 mmol) was added followed by acetic anhydride (132 μL, 1.40 mmol). The reaction was stirred at ambient temperature for 3 hours. The reaction was filtered and purified by reverse phase HPLC to give a diastereomeric mixture of the desired products.

### 2-(1-Acetyl-piperidin-3-yl)-4-[4-(4-cyclopropylcarbamoyl-phenyl)-thiazol-2-ylcarbamoyl]-thiazolidine-3-carboxylic acid benzyl ester (compound 7133)

Prepared from (R)-2-(1-acetyl-piperidin-3-yl)-thiazolidine-3,4-dicarboxylic acid 3-benzyl ester using experimental procedures described for compound 7080 (Example 80). MS: 634.2 (M+H⁺); H¹ NMR (DMSO-d₆): δ (ppm) 0.53-0.76 (m, 4H), 1.06-1.50 (m, 4H), 1.59-2.22 (m, 6H), 2.78-2.91 (m, 2H), 3.97-4.42 (m, 3H), 4.78-5.22 (m, 4H), 7.04-7.44 (m, 5H), 7.78-7.99 (m, 4H), 8.42-8.48 (m, 1H).

### Example 134

### 4-(4-carbamoyl-phenylcarbamoyl)-2-pyridin-4-yl-thiazolidine-3-carboxylic acid benzyl ester (Compound 7134)

Synthesized using experimental procedure for compound 7031 (Example 31). MS: 463.1 (M+H⁺).

### Example 135

### 4-[4-(2-Methoxy-ethylcarbamoyl)-phenylcarbamoyl]-2-pyridin-4-yl-thiazolidine-3-carboxylic acid benzyl ester (Compound 7135)

Synthesized using experimental procedure for compound 7031 (Example 31). MS: 521.2 (M+H⁺).

### Example 136

### 4-[3-(Phenethylcarbamoyl-methyl)-phenylcarbamoyl]-2-pyridin-4-yl-thiazolidine-3-carboxylic acid benzyl ester (Compound 7136)

Prepared using experimental procedure described for compound 7031 (Example 31). MS: 581.7 (M+H⁺)

### Example 137

### 4-[5-(4-Methoxy-benzyl)-thiazol-2-ylcarbamoyl]-2-pyridin-4-yl-thiazolidine-3-carboxylic acid benzyl ester (Compound 7137)

Prepared using experimental procedure described for compound 7031 (Example 31). MS: 547.1 (M+H⁺); H¹ NMR (DMSO-d₆): δ (ppm) 9.02 (d, 1H), 8.92 (d, 1H), 8.27 (d, 1H), 8.15 (d, 1H), 7.40 (s, 1H), 6.88-7.28 (m, 8H), 6.54 (d, 1H), 5.09 (m, 2H), 4.13 (d, 1H), 3.88 (m, 1H), 3.82 (d, 3H), 3.37 (t, 1H).

### Example 138

### 3-{4-[4-(2-Morpholin-4-yl-ethylcarbamoyl)-phenyl]-thiazol-2-ylcarbamoyl}-8-oxa-1-thia-4-aza-spiro[4.5]decane-4-carboxylic acid benzyl ester (Compound 7138)

### 8-Oxa-1-thia-4-aza-spiro[4.5]decane-3-carboxylic acid

L-cysteine (2.0 g, 12.7 mmol) was combined with tetrahydro-pyran-4-one (1.27 g, 12.7 mmol). This mixture was dissolved in 10 mL of 1:1 H₂O/EtOH and heated for 1 hour at 90° C. It was evaporated do dryness, and washed using diethyl ether. The resulting white powder was used in the next step without further purification.

### 8-Oxa-1-thia-4-aza-spiro[4.5]decane-3,4-dicarboxylic acid 4-benzyl ester

8-Oxa-1-thia-4-aza-spiro[4.5]decane-3-carboxylic acid, IS2423-59, (1.0 g, 4.9 mmol) was partially dissolved in 25 mL of dichloromethane. At 0° C., DIEA (0.85 ml), followed by benzyl chloroformate (0.7 mL, 4.9 mmol) was added to the solution. Reaction mixture was brought to room temperature and stirred at room temperature for 45 minutes. It was extracted using 10% citric acid and water. Organic layer was isolated, dried over Na₂SO₄ and evaporated to yield clear oil that was used in the next step without further purification.

### 3-{4-[4-(2-Morpholin-4-yl-ethylcarbamoyl)-phenyl]-thiazol-2-ylcarbamoyl}-8-oxa-1-thia-4-aza-spiro[4.5]decane-4-carboxylic acid benzyl ester (compound 7138)

8-Oxa-1-thia-4-aza-spiro[4.5]decane-3,4-dicarboxylic acid 4-benzyl ester, (330 mg, 0.98 mmol) was combined with (0.98 mmol, 373 mg) of HATU in 2.5 mL of DMF. To this solution was added DIEA (1.96 mmol, 0.34 mL) and mixture stirred at room temperature for 1 hour. 4-(2-amino-thiazol-4-yl)-N-(2-morpholin-4-ethyl)-benzamide (0.98 mmol, 319 mg) was then added to the reaction mixture which was stirred at 50° C. overnight. Reaction mixture was diluted using EtOAc and extracted using NaHCO₃ (sat). Organic phase was isolated and concentrated to yield crude product which was purified using reverse phase HPLC. MS: 652.2 (M+H⁺); H¹ NMR (Acetone-d₆): δ (ppm) 11.37 (brs s, 1H), 8.70 (br t, 1H), 7.97 (m. 5H), 7.63 (s, 1H), 7.26 (m, 4H), 5.39 (m, 1H), 5.12 (m, 3H), 4.49 (br s, 1H), 3.91 (m, 8H), 3.51 (m, 4H), 3.38 (m, 8H).

### Example 139

### 3-{4-[4-(Pyridin-3-ylcarbamoyl)-phenyl]-thiazol-2-ylcarbamoyl}-8-oxa-1-thia-4-aza-spiro-[4.5]decane-4-carboxylic acid benzyl ester (Compound 7139)

Synthesized using experimental procedure for compound 7138 (Example 138). MS: 616.7 (M+H⁺); H¹ NMR (Acetone-d₆): δ (ppm) 10.47 (s, 1H), 9.53 (d, 1H), 8.88 (d, 1H), 8.68 (d, 1H), 8.08 (m, 5H) 7.74 (s, 1H), 7.33 (m, 5H), 5.40 (m, 2H), 5.12 (m, 2H), 3.98 (m, 4H), 3.62 (m, 2H), 3.38 (m, 4H).

### Example 140

### 3-{4-[4-(Pyridin-3-ylcarbamoyl)-phenyl]-thiazol-2-ylcarbamoyl}-1,8-dioxa-4-aza-spiro[4.5]decane-4-carboxylic acid benzyl ester (Compound 7140)

### 1,8-Dioxa-4-aza-spiro[4.5]decane-3,4-dicarboxylic acid 4-benzyl ester 3-methyl ester

2-Benzyloxycarbonylamino-3-hydroxy-propionic acid methyl ester (2.0 g, 7.9 mmol) was combined with tetrahydro-pyran-4-one (790 mg, 7.9 mmol). This mixture was dissolved in 120 mL of dry toluene. Catalytic amount (50 mg) of p-toluenesulfonic acid was then added to the reaction mixture which was heated under reflux overnight. It was brought to room temperature, diluted using EtOAc, and extracted using NaHCO₃. Organic phase was isolated, dried over Na₂SO₄ and evaporated. The resulting clear oil was used in the next step without further purification. 1,8-Dioxa-4-aza-spiro[4.5]decane-3,4-dicarboxylic acid benzyl ester

1,8-Dioxa-4-aza-spiro[4.5]decane-3,4-dicarboxylic acid 4-benzyl ester 3-methyl ester, (1.0 g, 3.0 mmol) was dissolved in 10 mL of 3:1:1 mixture of THF/MeOH/H₂O. LiOH (6.0 mmol, 144 mg) was added to the solution which was then stirred at room temperature for 30 minutes. Organic solvent was evaporated. The remaining aqueous layer was extracted using EtOAc. Aqueous layer was isolated, acidified using 1M HCl, and extracted into EtOAc. Organic layer was washed with H₂O, dried over Na₂SO₄, and evaporated. The resulting white solid was used without further purification.

### 3-{4-[4-(Pyridin-3-ylcarbamoyl)-phenyl]-thiazol-2-ylcarbamoyl}-1,8-dioxa-4-aza-spiro[4.5]decane-4-carboxylic acid benzyl ester (Compound 7140)

1,8-Dioxa-4-aza-spiro[4.5]decane-3,4-dicarboxylic acid benzyl ester, IS2423-74, (0.51 mmol, 162.6 mg) was combined with (0.51 mmol, 194 mg) of HATU. This mixture was dissolved in 2.5 mL of DMF. To the solution was added DIEA (0.11 mmol, 0.3 mL) and reaction stirred at room temperature for 30 minutes. 4-(2-amino-thiazol-4-yl)-N-pyridin-3-ylbenzamide (0.51 mmol, 150 mg) was added to the reaction mixture which was then stirred at 50° C. overnight. It was diluted using EtOAc and extracted using NaHCO₃ (aq). Organic phase was isolated, dried over Na₂SO₄, and evaporated. This crude material was purified using reverse phase HPLC. MS: 600.2 (M+H⁺); H¹ NMR (DMSO-d₆): δ (ppm) 12.59 (s, 1H), 10.69 (s, 1H), 9.09 (d, 1H), 8.41 (m, 2H), 8.36 (m, 2H), 8.06 (m, 4H), 7.88 (s, 1H), 7.62 (m, 1H), 7.37 (s, 1H), 7.19 (m, 4H), 5.12 (m, 2H), 4.78 (m, 1H), 4.23 (m, 2H), 3.83 (m, 2H), 3.45 (m, 2H), 1.68 (m, 2H).

### Example 141

### 3-{4-[4-(2-Morpholin-4-yl-ethylcarbamoyl)-phenyl]-thiazol-2-ylcarbamoyl}-1,8-dioxa-4-aza-spiro[4.5]decane-4-carboxylic acid benzyl ester (Compound 7141)

Synthesized using procedure for compound 7140 (Example 140). MS: 636.2 (M+H); H¹ NMR (DMSO-d₆): δ (ppm) 12.56 (s, 1H), 9.89 (s, 1H), 8.77 (br t, 1H), 7.93 (m, 5H), 7.36 (s, 1H), 7.15 (m, 4H), 5.07 (m, 2H), 4.77 (m, 2H), 4.28-3.80 (m, 6H), 3.65-3.32 (m, 8H), 3.14 (m, 2H), 1.68 (dd, 2H).

### BIOLOGICAL EXAMPLES

### Example 1

### Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles et al. In vitro assays have been reported in Ferrari et al. J. of Vir., 73:1649-1654, 1999; Ishii et al., Hepatology, 29:1227-1235, 1999; Lohmann et al., J. of Bio. Chem., 274:10807-10815, 1999; and Yamashita et al., J. of Bio. Chem., 273:15479-15486, 1998.

### Replicon Assay

A cell line, ET (Huh-lucubineo-ET) was used for screening of compounds of the present invention for inhibiting HCV replication. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T12801; K1846T) (Krieger at al, 2001 and unpublished). The ET cells were grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×10⁴ cells/well in the 96 well plates and incubated for 24 hrs before adding testing compounds. Then the compounds were added to the cells to achieve a final concentration of 0.1 nM to 50 μM and a final DMSO concentration of 0.5%. Luciferase activity were measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication was plotted relative to no compound control. Under the same condition, cytotoxicity of the compounds was determined using cell proliferation reagent, WST-1 (Roche, Germany). The compounds showing antiviral activities, but no significant cytotoxicities were chosen to determine EC₅₀ and TC₅₀. For these determinations, a 10 point 2-fold serial dilution for each compound was used, which spans a concentration range of 1000 fold. EC₅₀ and similarly TC₅₀ values were calculated by fitting % inhibition at each concentration to the following equation:

% inhibition=100%/[(IC50/[I])^{b}+1]

where b is Hill's coefficient.

Preferably, when tested at 100 μM the compounds of this invention will exhibit a % inhibition of at least 30% and more preferably a % inhibition of at least 50%.

When tested at 5 μM, the compounds in Table 1 were found to have the indicated percent inhibition values shown in Table 2. Compounds of Table 1 with % inhibition of less than 1% are not included in Table 2, but may have greater activity when tested at higher concentrations. In some preferred embodiments compounds of Formula I will have a % inhibition of at least 20% when tested at 5 μM. In other embodiments the compounds of Formula I will have a % inhibition of at least 50% when tested at 5 μM.

TABLE 2

| Compound # | % inhibition at 5 μM |
|---|---|
| 7002 | 97.5 |
| 7003 | 100.0 |
| 7004 | 99.9 |
| 7005 | 97.8 |
| 7006 | 98.4 |
| 7007 | 100.0 |
| 7008 | 100.0 |
| 7009 | 99.9 |
| 7010 | 98.1 |
| 7011 | 98.8 |
| 7012 | 98.8 |
| 7013 | 99.7 |
| 7014 | 97.7 |
| 7015 | 100.0 |
| 7016 | 98.5 |
| 7017 | 100.0 |
| 7018 | 47.6 |
| 7019 | 98.3 |
| 7020 | 99.5 |
| 7021 | 100.0 |
| 7022 | 100.0 |
| 7023 | 78.5 |
| 7024 | 99.7 |
| 7025 | 100.0 |
| 7026 | 89.1 |
| 7027 | 6.9 |
| 7028 | 100.0 |
| 7029 | 99.8 |
| 7030 | 99.9 |
| 7031 | 99.8 |
| 7032 | 99.2 |
| 7033 | 100.0 |
| 7034 | 98.7 |
| 7035 | 98.9 |
| 7036 | 99.8 |
| 7037 | 100.0 |
| 7038 | 48.4 |
| 7039 | 100.0 |
| 7040 | 89.9 |
| 7041 | 100.0 |
| 7042 | 100.0 |
| 7043 | 100.0 |
| 7044 | 98.0 |
| 7045 | 100.0 |
| 7046 | 99.1 |
| 7047 | 99.6 |
| 7048 | 99.3 |
| 7049 | 98.8 |
| 7050 | 100.0 |
| 7051 | 99.4 |
| 7052 | 98.9 |
| 7053 | 99.6 |
| 7054 | 94.1 |
| 7055 | 99.1 |
| 7056 | 99.9 |
| 7057 | 100.0 |
| 7058 | 99.9 |
| 7064 | 98.8 |
| 7080 | 100.0 |
| 7087 | 99.6 |
| 7095 | 99.9 |
| 7109 | 100.0 |
| 7122 | 100.0 |
| 7121 | 99.8 |
| 7108 | 99.9 |
| 7065 | 92.0 |
| 7081 | 100.0 |
| 7088 | 99.8 |
| 7078 | 35.6 |
| 7096 | 99.9 |
| 7120 | 100.0 |
| 7107 | 10.0 |
| 7066 | 6.9 |
| 7077 | 2.1 |
| 7111 | 43.0 |
| 7131 | 100.0 |
| 7119 | 1.2 |
| 7106 | 32.9 |
| 7090 | 99.7 |
| 7076 | 3.9 |
| 7098 | 99.9 |
| 7112 | 64.2 |
| 7125 | 100.0 |
| 7118 | 99.9 |
| 7105 | 74.2 |
| 7068 | 6.0 |
| 7084 | 14.2 |
| 7091 | 100.0 |
| 7075 | 21.3 |
| 7099 | 100.0 |
| 7113 | 86.5 |
| 7126 | 93.8 |
| 7133 | 100.0 |
| 7117 | 42.4 |
| 7104 | 100.0 |
| 7069 | 98.8 |
| 7085 | 99.6 |
| 7092 | 89.4 |
| 7074 | 99.0 |
| 7100 | 80.1 |
| 7114 | 100.0 |
| 7127 | 25.7 |
| 7134 | 13.6 |
| 7116 | 84.9 |
| 7103 | 99.8 |
| 7070 | 96.1 |
| 7093 | 100.0 |
| 7073 | 65.3 |
| 7101 | 98.4 |
| 7115 | 98.0 |
| 7128 | 97.5 |
| 7135 | 22.1 |
| 7102 | 100.0 |
| 7071 | 100.0 |
| 7136 | 90.0 |
| 7094 | 88.0 |
| 7072 | 80.8 |
| 7137 | 17.1 |
| 7138 | 100.0 |
| 7139 | 100.0 |
| 7140 | 99.9 |
| 7141 | 100.0 |

### FORMULATION EXAMPLES

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration.

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co.) | 1.0 | g |
| flavoring | 0.035 | mL |
| colorings | 0.5 | mg |
| distilled water | q.s. to 100 | mL |

### Example 4

### Injectable Formulation

The following ingredients are mixed to form an injectable formulation.

| Ingredient | Quantity per tablet, mg | |
|---|---|---|
| Compound of the invention | 0.2 mg-20 mg | |
| sodium acetate buffer solution, 0.4 M | 2.0 | mL |
| HCl (1N) or NaOH (1N) | q.s. to suitable pH | |
| water (distilled, sterile) | q.s. to 20 | mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | balance |

What is claimed is:

1. A compound of Formula (I) or a stereoisomer, tautomer, pharmaceutically acceptable salt, or prodrug thereof, wherein: 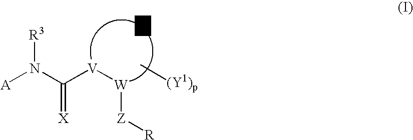
A is a 3-13 membered ring optionally substituted with —(R²)ₘ wherein said ring is selected from the group consisting of cycloalkyl, heterocyclic, aryl, and heteroaryl;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 0, 1, 2, or 3;
R³ is selected from the group consisting of hydrogen, alkyl, substituted alkyl, cycloalkyl, and substituted cycloalkyl;
X is O or S;
T is C₂-C₆ alkylene or C₁-C₅ heteroalkylene and forms a 4-8 membered ring with V and W;
V and W are both CH, or one of V or W is CH and the other of V or W is N;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

2. A compound of claim 1 wherein A is selected from the group consisting of

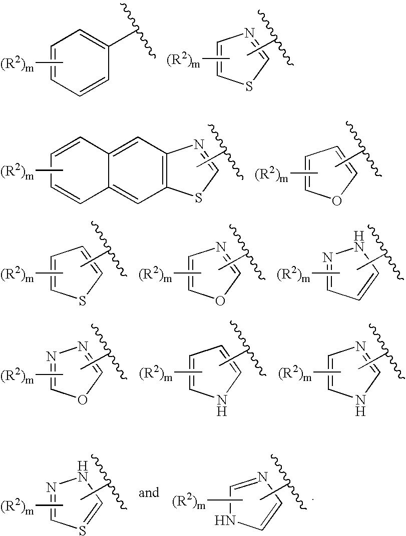

3. A compound of claim 1 wherein at least one of R² is R⁴—L- wherein R⁴ is selected from the group consisting of aryl, substituted aryl, heteroaryl, substituted heteroaryl, heterocyclic, and substituted heterocyclic; and L, defined in the R⁴—L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R⁴ is heterocyclic or substituted heterocyclic.

4. A compound of claim 3 wherein R⁴ is substituted phenyl.

5. A compound of claim 1 wherein V is C and W is N.

6. A compound of claim 1 wherein Z is C(O).

7. A compound of claim 1 wherein R is OCH₂R¹ and R¹ is phenyl or substituted phenyl.

8. A compound of claim 1 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

9. A compound of claim 1 wherein p is 2 and two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing T, V, and W form a spiro ring system.

10. A compound of claim 1 having Formula (II) or a stereoisomer, tautomer, or pharmaceutically acceptable salt thereof, wherein: 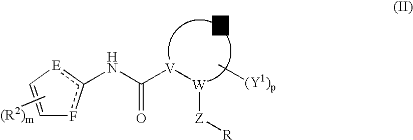
one of E or F is —N═ and the other of E or F is —O—, —S—, or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
T is C₂-C₆ alkylene or C₁-C₅ heteroalkylene and forms a 4-8 membered ring with V and W;
V and W are both CH, or one of V or W is CH and the other of V or W is N;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

11. A compound of claim 10 wherein at least one of R² is R⁴—L- wherein R⁴ is selected from the group consisting of aryl, substituted aryl, heteroaryl, substituted heteroaryl, heterocyclic, and substituted heterocyclic; and L, defined in the R⁴-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R⁴ is heterocyclic or substituted heterocyclic.

12. A compound of claim 11 wherein R⁴ is substituted phenyl.

13. A compound of claim 10 wherein V is C and W is N.

14. A compound of claim 10 wherein Z is C(O).

15. A compound of claim 10 wherein R is OCH₂R¹ and R¹ is phenyl or substituted phenyl.

16. A compound of claim 10 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

17. A compound of claim 10 wherein p is 2 and two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing T, V, and W form a spiro ring system.

18. A compound of claim 1 having Formula (III) or a stereoisomer, tautomer, pharmaceutically acceptable salt, or prodrug thereof, wherein: 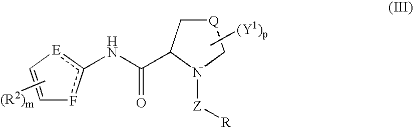
one of E or F is —N═ and the other of E or F is —O—, —S—, or —NH—;
each R² is independently selected from the group consisting of alkyl, substituted alkyl, alkoxy, substituted alkoxy, acyl, acylamino, acyloxy, amino, substituted amino, aminocarbonyl, aryl, substituted aryl, carboxyl, carboxyl ester, cycloalkyl, substituted cycloalkyl, halo, hydroxy, heteroaryl, substituted heteroaryl, heterocyclic, substituted heterocyclic, nitro, thiol, alkylthio, and substituted alkylthio;
m is 1 or 2;
Q is selected from the group consisting of CH₂, CH(Y¹), C(Y¹)(Y¹), S, and O;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

19. A compound of claim 18 wherein at least one of R² is R⁴—L- wherein R⁴ is selected from the group consisting of aryl, substituted aryl, heteroaryl, substituted heteroaryl, heterocyclic, and substituted heterocyclic; and L, defined in the R⁴-L- orientation, is selected from the group consisting of a bond, —O—, —S—, —CH₂—, —CH₂CH₂—, —SCH₂—, —C(O)—, —C(S)—, —NHC(O)—, —C(O)NH—, —SO₂—, —SO₂NH—, —SO₂CH₂—, —OCH₂—, —CH₂CH₂NHC(O)—, —CH₂CH₂NHC(O)CH₂—, —NHN═C(CH₃CH₂OCO)—, —NHSO₂—, ═CH—, —NHC(O)CH₂S—, —NHC(O)CH₂C(O)—, spirocycloalkyl, —C(O)CH₂S—, and —C(O)CH₂O— provided that when L is ═CH—, R⁴ is heterocyclic or substituted heterocyclic.

20. A compound of claim 19 wherein R⁴ is substituted phenyl.

21. A compound of claim 20 wherein Z is C(O).

22. A compound of claim 20 wherein R is OCH₂R¹ and R¹ is phenyl or substituted phenyl.

23. A compound of claim 20 wherein Q is S, CH₂, or O.

24. A compound of claim 20 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

25. A compound of claim 18 wherein p is 2 and two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing Q form a spiro ring system.

26. A compound of claim 25 having formula (IIIa) or a stereoisomer, tautomer, pharmaceutically acceptable salt, or prodrug thereof, wherein: 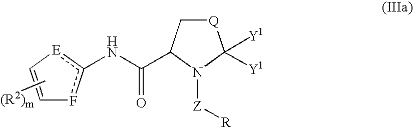
two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing Q form a spiro ring system; and
R², m, E, F, Q, Z, R, and Y² are as defined for Formula (III).

27. A compound of claim 1 having Formula (IV) or a stereoisomer, tautomer, pharmaceutically acceptable salt or prodrug thereof, wherein: 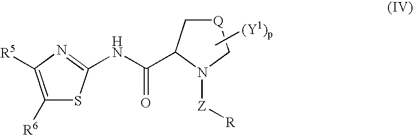
R⁵ is selected from the group consisting of substituted cycloalkyl, substituted phenyl, substituted heterocyclic, and substituted heteroaryl;
R⁶ is independently selected from the group consisting of hydrogen, alkyl, substituted alkyl, and halo;
Q is selected from the group consisting of CH₂, CH(Y¹), C(Y¹)(Y¹), S, and O;
p is 1 or 2;
each Y¹ is independently selected from the group consisting of alkyl, substituted alkyl, aryl, substituted aryl, heteroaryl, substituted heteroaryl, cycloalkyl, substituted cycloalkyl, heterocyclic, substituted heterocyclic, and ═CH₂; or optionally when p is 2, the other of Y¹ is selected from the group consisting of halo, hydroxy, alkoxy, and substituted alkoxy, or two Y¹ groups together with the atoms to which they are bound form a phenyl, 4-7 membered cycloalkyl, or 4-7 membered heterocyclic ring wherein the phenyl, cycloalkyl, or heterocyclic ring is itself optionally substituted with 1 to 2 Y² groups;
Y² is independently selected from the group consisting of alkyl, substituted alkyl, halo, oxo, hydroxy, carboxyl, carboxyl ester, cyano, and alkoxy with the proviso that Y² is not oxo when the ring to which it is attached is phenyl;
Z is selected from the group consisting of C(O), C(S), and —SO₂—;
R is selected from the group consisting of R¹, OR¹, OCH₂R¹, and NR^{1a}R¹;
R¹ is selected from the group consisting of alkyl, substituted alkyl, cycloalkyl, substituted cycloalkyl, heterocyclic, substituted heterocyclic, aryl, substituted aryl, heteroaryl, and substituted heteroaryl; and
R^{1a} is selected from the group consisting of hydrogen, alkyl, and substituted alkyl.

28. A compound of claim 27 wherein R⁵ is substituted phenyl.

29. A compound of claim 28 wherein said substituted phenyl is substituted with one to three groups independently selected from alkyl, substituted alkyl, alkenyl, substituted alkenyl, alkoxy, substituted alkoxy, aryloxy, substituted aryloxy, alkylthio, substituted alkyl thio, acyl, acylamino, acyloxy, amino, substituted amino, aminocarbonyl, aminocarbonylamino, aminocarbonyloxy, aryl, substituted aryl, carboxyl, carboxyl ester, cyano cycloalkyl, substituted cycloalkyl, halo, hydroxy, heteroaryl, substituted heteroaryl, heterocyclic, substituted heterocyclic, nitro, thiol, alkylthio, and substituted alkylthio.

30. A compound of claim 27 wherein Z is C(O).

31. A compound of claim 27 wherein R is OCH₂R¹ and R¹ is phenyl or substituted phenyl.

32. A compound of claim 27 wherein Q is S, CH₂, or O.

33. A compound of claim 27 wherein p is 1 and Y¹ is selected from the group consisting of substituted alkyl, aryl, substituted aryl, heteroaryl, substituted heteroaryl, cycloalkyl, substituted cycloalkyl, heterocyclic, and substituted heterocyclic.

34. A compound of claim 27 wherein p is 2 and two Y¹ groups together form a 4-7 membered cycloalkyl or 4-7 membered heterocyclic ring wherein the cycloalkyl or heterocyclic ring is itself optionally substituted with 1 to 2 Y² groups, and wherein said 4-7 membered cycloalkyl or 4-7 membered heterocyclic ring together with the ring containing Q form a spiro ring system.

35. A compound of claim 1 or a stereoisomer, tautomer, pharmaceutically acceptable salt, or prodrug thereof selected from Table 1.

36. A pharmaceutical composition comprising a pharmaceutically acceptable carrier and a therapeutically effective amount of a compound, stereoisomer, tautomer, pharmaceutically acceptable salt, or prodrug thereof of claim 1.

37. A method for treating a viral infection in a patient mediated at least in part by a virus in the Flaviviridae family of viruses which method comprises administering to the patient a compound, stereoisomer, tautomer, pharmaceutically acceptable salt, or prodrug thereof of claim 1.

38. The method of claim 37 wherein said viral infection is a hepatitis C mediated viral infection.

39. The method of claim 37 in combination with the administration of a therapeutically effective amount of one or more agents active against hepatitis C virus.

40. The method of claim 38 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

41. The method of claim 38 wherein said agent active against hepatitis C virus is interferon.
